SEQUENCE LISTING (1 sequence)

SEQ ID NO: 1 - protein, 153 aa, Artificial sequence
  engineered human IL-1 receptor antagonist (IL-1Ra) agent
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

(19) United States
(12) Patent Application Publication
(10) Pub. No.: US 2023/0133243 A1
(43) Pub. Date: May 4, 2023

(54) CONDITIONING REGIMENS FOR IN VIVO GENE THERAPY

(71) Applicants: Fred Hutchinson Cancer Center, Seattle, WA (US); University of Washington, Seattle, WA (US)

(72) Inventors: Andre Lieber, Seattle, WA (US); Hans-Peter Kiem, Seattle, WA (US)

(73) Assignees: Fred Hutchinson Cancer Center, Seattle, WA (US); University of Washington, Seattle, WA (US)

(21) Appl. No.: 17/995,687

(22) PCT Filed: Apr. 12, 2021
(86) PCT No.: PCT/US2021/026873

§ 371 Date: Oct. 6, 2022

Related U.S. Application Data
(60) Provisional application No. 63/121,777, filed on Dec. 4, 2020.
(60) Provisional application No. 63/009,218, filed on Apr. 13, 2020.

(51) Int. Cl. A61K 48/00; A61K 38/20; A61K 39/395; A61K 31/436; A61K 31/573; A61K 38/19; A61K 31/395; A61P 37/06; A61P 7/06

(52) U.S. Cl. CPC A61K 48/0083; A61K 38/2006; A61K 39/3955; A61K 31/436; A61K 31/573; A61K 38/193; A61K 31/395; A61P 37/06; A61P 7/06

(57) ABSTRACT

The present disclosure provides, among other things, immune suppression regimens for in vivo gene therapy and uses thereof. In various embodiments of the present disclosure, in vivo gene therapy includes delivery of at least one exogenous coding nucleic acid sequence to a stem cell of the subject. Success of in vivo gene therapy can be inhibited or reduced by immunotoxicity. The present disclosure provides compositions and methods, including among other things immune suppression regimens, that reduce immunotoxicity of in vivo gene therapy, e.g., in vivo gene therapy including administration of a viral gene therapy vector to a subject.

### CROSS-REFERENCE TO RELATED APPLICATION

This is the 371 National Phase of co-pending international application No. PCT/US2021/026873, filed Apr. 12, 2021, which application claims priority to and the benefit of the earlier filing date of U.S. Provisional Application No. 63/121,777, filed on Dec. 4, 2020; and of U.S. Provisional Application No. 63/009,218, filed Apr. 13, 2020. Each of these prior applications is incorporated by reference herein in its entirety.

### GOVERNMENT SUPPORT

This invention was made with government support under HL136135, HL128288 and HL130040 awarded by the National Institutes of Health. The government has certain rights in this invention.

### BACKGROUND

Many existing gene therapies deliver a nucleic acid sequence of interest to a subject via ex vivo engineered cells. Certain such methods have a variety of drawbacks, including, without limitation, the cost and technical complexity of therapies based on ex vivo engineered cells, including the need for sophisticated facilities to isolate, modify, expand, and isolate ex vivo engineered cells. Additional challenges of certain therapies based on ex vivo engineered cells include difficulty achieving engraftment in recipients. To provide one example, ex vivo therapies that include high-dose chemotherapy to facilitate engraftment of engineered cells are particularly unsuitable for treatment of hemoglobinopathies. For at least these reasons, there is a need for in vivo gene therapy strategies and protocols that enhance the safety and/or efficacy of in vivo gene therapies.

### SUMMARY

The present disclosure provides, among other things, immune suppression regimens for in vivo gene therapy and uses thereof. As disclosed herein, in vivo gene therapy includes therapies in which at least one exogenous coding nucleic acid sequence is delivered to cells of a subject without removal of the cells from the subject. In various embodiments of the present disclosure, in vivo gene therapy includes delivery of at least one exogenous coding nucleic acid sequence to a stem cell of the subject. In various embodiments of the present disclosure, in vivo gene therapy includes administering to a subject a viral vector that includes a nucleic acid sequence that encodes an exogenous coding nucleic acid sequence. Success of in vivo gene therapy can be inhibited or reduced by immunotoxicity, i.e., induction by a therapeutic agent or regimen, such as a therapeutic agent or regimen that includes a viral gene therapy vector, of an immune response in a subject that is counterproductive to treatment and/or the well-being of the subject. In some cases, immunotoxicity includes an inflammatory response and/or a cytokine response. The present disclosure provides compositions and methods, including among other things immune suppression regimens, that reduce immunotoxicity of in vivo gene therapy, e.g., in vivo gene therapy including administration of a viral gene therapy vector to a subject.

Particular embodiments include a method of transducing stem cells of a mammalian subject without removal of the stem cells from the subject, the method including delivering a viral gene therapy vector to a subject having been administered an immune suppression regimen including an inflammatory signal inhibitor.

Particular embodiments include a method of in vivo gene therapy in a mammalian subject, the method including: (i) administering to the subject an immune suppression regimen including an inflammatory signal inhibitor; and (ii) administering to the subject at least one dose of a viral gene therapy vector.

Among other aspects, provided herein is the first demonstration that a combination of anakinra (Kineret®) and tocilizumab (Actemra®) with dexamethasone was fully able to blunt the innate immune response to HDAd5/35++. This documents a role for IL-1 and IL-6 in driving the innate response to these vectors.

In at least one aspect, the present disclosure provides a method of in vivo gene therapy in a mammalian subject, the method including: (i) administering to the subject an immune suppression regimen including an inflammatory signal inhibitor; and (ii) administering to the subject at least one dose of a viral gene therapy vector.

In at least one aspect, the present disclosure provides a method of transducing stem cells of a mammalian subject without removal of the stem cells from the subject, the method including delivering a viral gene therapy vector to a subject having been administered an immune suppression regimen including an inflammatory signal inhibitor.

In various embodiments, the inflammatory signal inhibitor is an interleukin-1 (IL-1) signal inhibitor, optionally where the IL-1 signal inhibitor is an IL-1 receptor (IL-1R) antagonist.

In various embodiments, the IL-1R antagonist is anakinra.

In various embodiments, the immune suppression regimen further includes an interleukin 6 (IL-6) receptor antagonist.

In various embodiments, the IL-6 receptor antagonist is tocilizumab.

In various embodiments, the immune suppression regimen further includes a corticosteroid.

In various embodiments, the corticosteroid is dexamethasone.

In various embodiments, the immune suppression regimen further includes a calcineurin inhibitor.

In various embodiments, the calcineurin inhibitor is tacrolimus.

In various embodiments, the immune suppression regimen further includes a TNF-α signal inhibitor.

In various embodiments, the TNF-α signal inhibitor is selected from the group including etanercept, infliximab, adalimumab, certolizumab, pegol, and golimumab.

In various embodiments, the immune suppression regimen further includes a JAK signal inhibitor.

In various embodiments, the JAK signal inhibitor is selected from the group including baricitinib, tofacitinib, ruxolitinib, and filgotinib.

In various embodiments, the administering of the immune suppression regimen includes administering an IL-1 receptor antagonist to the subject: (i) on the day prior to administration of a first dose of the vector; (ii) on the day of administration of a first dose of the vector, optionally including at least one dose of IL-1 receptor antagonist 1 to 3 hours prior to administration of the first dose of the vector; (iii) on the day of administration of one or more subsequent doses of the vector, optionally including at least one dose of IL-1 receptor antagonist 1 to 3 hours prior to administration of the one or more subsequent doses of the vector; (iv) on each day between the day of administration of a first dose of the vector and the day of administration of a last dose of the vector; and/or (v) on each of one, two, or more days after the day of administration of a last dose of the vector; optionally where the IL-1 receptor antagonist is anakinra.

In various embodiments, the administering of the immune suppression regimen includes administering to the subject a single dose of IL-1 receptor antagonist per day or a plurality of doses of IL-1 receptor antagonist per day, optionally where the IL-1 receptor antagonist is anakinra.

In various embodiments, the administering of the immune suppression regimen includes administering to the subject 0.01 to 20 mg/kg/day anakinra, optionally where the administration is intravenous or subcutaneous.

In various embodiments, the administering of the immune suppression regimen includes administering to the subject 10 to 200 mg/day anakinra, optionally where the administration is intravenous or subcutaneous.

In various embodiments, the administering of the immune suppression regimen includes administering an IL-6 receptor antagonist to the subject: (i) on the day prior to administration of a first dose of the vector; (ii) on the day of administration of a first dose of the vector, optionally including at least one dose of IL-6 receptor antagonist no more than 1 hour prior to administration of the first dose of the vector; (iii) on the day of administration of one or more subsequent doses of the vector, optionally including at least one dose of IL-6 receptor antagonist no more than 1 hour prior to administration of the one or more subsequent doses of the vector; (iv) on each day between the day of administration of a first dose of the vector and the day of administration of a last dose of the vector; and/or (v) on each of one, two, or more days after the day of administration of a last dose of the vector; optionally where the IL-6 receptor antagonist is tocilizumab.

In various embodiments, the administering of the immune suppression regimen includes administering to the subject a single dose of IL-6 receptor antagonist per day or a plurality of doses of IL-6 receptor antagonist per day, optionally where the IL-6 receptor antagonist is tocilizumab.

In various embodiments, the administering of the immune suppression regimen includes administering to the subject 1-15 mg/kg/day tocilizumab or 5-200 mg/day tocilizumab, optionally where the administration is intravenous.

In various embodiments, the administering of the immune suppression regimen includes administering a corticosteroid to the subject: (i) on the day prior to administration of a first dose of the vector; (ii) on the day of administration of a first dose of the vector; (iii) on the day of administration of one or more subsequent doses of the vector; (iv) on each day between the day of administration of a first dose of the vector and the day of administration of a last dose of the vector; and/or (v) on each of one, two, or more days after the day of administration of a last dose of the vector; optionally where the corticosteroid is dexamethasone, prednisone, prednisolone, methylprednisolone, triamcinolone, paramethasone, or betamethasone.

In various embodiments, the administering of the immune suppression regimen includes administering to the subject a single dose of corticosteroid per day or a plurality of doses of corticosteroid per day, optionally where the corticosteroid is dexamethasone.

In various embodiments, the administering of the immune suppression regimen includes administering to the subject 0.1-10 mg/kg/day dexamethasone, optionally where the administration is intravenous, oral, or intramuscular.

In various embodiments, the administering of the immune suppression regimen includes administering a calcineurin inhibitor to the subject: (i) on each of the four days prior to administration of a first dose of the vector; (ii) on the day of administration of a first dose of the vector; (iii) on the day of administration of one or more subsequent doses of the vector; and/or (iv) on each day between the day of administration of a first dose of the vector and the day of administration of a last dose of the vector; and/or (v) on each of one, two, or more days after the day of administration of a last dose of the vector; optionally where the calcineurin inhibitor is tacrolimus.

In various embodiments, the administering of the immune suppression regimen includes administering to the subject a single dose of calcineurin inhibitor per day or a plurality of doses of calcineurin inhibitor per day, optionally where the calcineurin inhibitor is tacrolimus.

In various embodiments, the administering of the immune suppression regimen includes administering to the subject 0.001-0.1 mg/kg/day tacrolimus, optionally where the administration is subcutaneous.

In various embodiments, the method (i) does not cause a significant increase in the amount of one or more of IFN-g, TNF, IL-2, IL-4, IL-5, or IL-6; or (ii) causes a significantly smaller increase in the amount of one or more of IFN-g, TNF, IL-2, IL-4, IL-5, or IL-6 as compared to a control that does not include one or more immune suppression agents, optionally where the control does not include one or more immune suppression agents selected from (a) the inflammatory signal inhibitor; (b) the IL-6 receptor antagonist; (c) the corticosteroid; and (d) the calcineurin inhibitor; optionally where the amount is measured by ELISA or a cytokine bead array.

In various embodiments, the method further includes administering to the subject a stem cell mobilization regimen.

In various embodiments, the vector includes a nucleic acid sequence that encodes a selectable marker, optionally where the selectable marker is MGMT^{P140K}.

In various embodiments, the method includes administering to the subject a selecting agent, optionally where the selectable marker is MGMT^{P140K} and the selecting agent is O⁶BG/BCNU.

In various embodiments, the selecting agent is administered to the subject in one or more doses, optionally where a first dose of the selecting agent is administered to the subject about 1 week, 2 weeks, 3 weeks, 4 weeks, 5 weeks, 6 weeks, 7 weeks, 8 weeks, 9 weeks, and/or 10 weeks after administration of a first dose of the vector to the subject.

In various embodiments, the vector is administered to the subject by injection, optionally where the injection is intravenous or subcutaneous.

In various embodiments, at least a first dose of the vector includes at least 1E10, 1E11, or 1E12 viral particles per kilogram (vp/kg).

In various embodiments, the vector is administered at a total dosage of at least 1E10, 1E11, 1E12, 2E12, or 3E12 vp/kg.

In various embodiments, the vector is an adenoviral vector, adeno-associated viral vector, herpes simplex viral vector, retroviral vector, lentiviral vector, alphaviral vector, flaviviral vector, rhabdoviral vector, measles viral vector, Newcastle disease viral vector, poxviral vector, or picornaviral vector.

In various embodiments, the vector is an adenoviral vector.

In various embodiments, the vector is a group B adenoviral vector.

In various embodiments, the vector is, or is derived from, an Ad5/35 or Ad35 adenoviral vector, optionally where the vector is an Ad35++ or Ad5/35++ adenoviral vector.

In various embodiments, the vector is a replication incompetent vector, optionally where the replication incompetent vector is a helper-dependent adenoviral vector.

In various embodiments, viral gene therapy vector includes a nucleic acid including a therapeutic payload, and where the method further includes administering to the subject a support vector encoding an agent that facilitates integration of the therapeutic payload into a target cell genome.

In various embodiments, the support vector is administered to the subject together with the viral gene therapy vector.

In various embodiments, the support vector is administered at a total dosage of 1E9 to 1E14 viral particles per kilogram (vp/kg).

In various embodiments, the viral gene therapy vector includes a nucleic acid including a therapeutic payload, and where the method causes delivery of the therapeutic payload to stem cells, optionally where delivery of the therapeutic payload includes integration of the therapeutic payload into the genomes of the stem cells.

In various embodiments, the viral gene therapy vector includes a nucleic acid including a protein-encoding therapeutic payload, and, after administration of the vector to the subject, at least about 70%, about 80%, or about 90% of PBMCs of the subject express the protein.

In various embodiments, the subject is a human subject.

In various embodiments, the human subject suffers from sickle cell anemia, thalassemia, thalassemia intermedia, hemophilia A, hemophilia B, von Willebrand Disease, Factor V Deficiency, Factor VII Deficiency, Factor X Deficiency, Factor XI Deficiency, Factor XII Deficiency, Factor XIII Deficiency, Bernard-Soulier Syndrome, Gray Platelet Syndrome.

In various embodiments, the dosing regimen of one or more immune suppression agents of the immune suppression regimen is increased in unit dose, daily dose, total dose, frequency of doses, and/or total number of doses based on the measured level of an immunotoxicity biomarker in the subject or a sample from the subject after administration of at least one dose of the viral gene therapy vector, where the dosing regimen of the one or more immune suppression agents, is increased if the measured level is indicative of immunotoxicity.

In various embodiments, the immunotoxicity biomarker is selected from the group including IL-Iβ, IL-2, IL-3, IL-4, IL-5, IL-6, IL-8, IL-10, IL-12, IL-13, IL-15, IL-17, IL-23, IL-27, IL-30, IL-36, IL-1Ra, IL-2R, IFN-α, IFN-b, IFN-γ, MIP-Ia, MIP-Iβ, MCP-1, TNF-α, TNF-8 GM-CSF, G-CSF, CXCL9, CXCL10, VEGF, RANTES, EGF, HGF, FGF-6, CD40, CD40L, C-reactive protein, procalcitonin, ferritin, D-dimer, total population of lymphocytes, subpopulations of lymphocytes, subject temperature, and a combination thereof.

In various embodiments, the dosing regimen of one or more immune suppression agents of the immune suppression regimen is increased in unit dose, daily dose, total dose, frequency of doses, and/or total number of doses based on the measured level of antibodies to the viral gene therapy vector in the subject or a sample from the subject after administration of at least one dose of the viral gene therapy vector, where the dosing regimen of the one or more immune suppression agents, is increased if the measured level is indicative of immunotoxicity, optionally where the measured level is an antibody titer, and optionally where the antibodies are neutralizing antibodies.

In various embodiments, the dosing regimen of the one or more immune suppression agents of the immune suppression regimen includes a dosing regimen of one or more of (i) an interleukin-1 (IL-1) signal inhibitor, optionally where the IL-1 signal inhibitor is anakinra; (ii) an IL-6 signal inhibitor, optionally where the IL-6 signal inhibitor is tocilizumab; (iii) a corticosteroid, optionally where the corticosteroid is dexamethasone; and (iv) a calcineurin inhibitor, optionally where the calcineurin inhibitor is tacrolimus.

### Definitions

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Affinity: As used herein, “affinity” refers to the strength of the sum total of non-covalent interactions between a particular binding agent (e.g., a viral vector), and/or a binding moiety thereof, with a binding target (e.g., a cell). Unless indicated otherwise, as used herein, “binding affinity” refers to a 1:1 interaction between a binding agent and a binding target thereof (e.g., a viral vector with a target cell of the viral vector). Those of skill in the art appreciate that a change in affinity can be described by comparison to a reference (e.g., increased or decreased relative to a reference), or can be described numerically. Affinity can be measured and/or expressed in a number of ways known in the art, including equilibrium dissociation constant (K_{D}) and/or equilibrium association constant (K_{A}). K_{D} is the quotient of k_{off}/kₒₙ, whereas K_{A} is the quotient of kₒₙ/k_{off}, where kₒₙ refers to the association rate constant of, e.g., viral vector with target cell, and k_{off} refers to the dissociation of, e.g., viral vector from target cell. The kₒₙ and k_{off} can be determined by techniques known to those of skill in the art.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Antibody: As used herein, the term “antibody” refers to a polypeptide that includes canonical immunoglobulin sequence elements sufficient to confer specific binding to a particular target antigen. As is known in the art, intact antibodies as produced in nature are 150 kD tetrameric agents made up of two identical heavy chain polypeptides (about 50 kD each) and two identical light chain polypeptides (about 25 kD each) that associate with each other into what is commonly referred to as a “Y-shaped” structure. Each heavy chain is made up of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is made up of two domains—an amino-terminal variable (VL) domain, followed by a carboxy-terminal constant (CL) domain, separated from one another by another “switch.” Intact antibody tetramers are made up of two heavy chain-light chain dimers in which the heavy and light chains are linked to one another by a single disulfide bond, and two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®; Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, therapeutic agent, fused polypeptide, or other pendant group such as poly-ethylene glycol).

Antibody fragment: As used herein, an “antibody fragment” refers to a portion of an antibody or antibody agent as described herein, and typically refers to a portion that includes an antigen-binding portion or variable region thereof. An antibody fragment can be produced by any means. For example, in some embodiments, an antibody fragment can be enzymatically or chemically produced by fragmentation of an intact antibody or antibody agent. Alternatively, in some embodiments, an antibody fragment can be recombinantly produced (i.e., by expression of an engineered nucleic acid sequence. In some embodiments, an antibody fragment can be wholly or partially synthetically produced. In some embodiments, an antibody fragment (particularly an antigen-binding antibody fragment) can have a length of at least about 50, 60, 70, 80, 90, 100, 110, 120, 130, 140, 150, 160, 170, 180, 190 amino acids or more, in some embodiments at least about 200 amino acids.

Associated with: Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Between or From: As used herein, the term “between” refers to content that falls between indicated upper and lower, or first and second, boundaries, inclusive of the boundaries. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents; indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Biomarker. As used herein, the term “biomarker,” consistent with its use in the art, refers to an entity, condition, or activity whose presence, level, or form, correlates with a particular biological event or state of interest, so that it is considered to be a “marker” of that event or state. To give but a few examples of biomarkers, in some embodiments, a biomarker can be or include a marker for a particular disease, disorder or condition, or can be a marker for qualitative or quantitative probability that a particular disease, disorder or condition can develop, occur, or reoccur, e.g., in a subject. In some embodiments, a biomarker can be or include a marker for a particular therapeutic outcome, or qualitative of quantitative probability thereof. Thus, in various embodiments, a biomarker can be predictive, prognostic, and/or diagnostic, of the relevant biological event or state of interest. A biomarker can be an entity of any chemical class. For example, in some embodiments, a biomarker can be or include a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, and the like). In some embodiments, a biomarker is an activity such as expression of a product encoded by a gene or signaling through a biological pathway. Those of skill in the art will appreciate that a biomarker may be individually determinative of a particular biological event or state of interest or may represent or contribute to a determination of the statistical probability of a particular biological event or state of interest. In some embodiments, a biomarker is highly specific in that it reflects a high probability of a particular status of the biological event or state of interest. In some instances, there is an inverse relationship between specificity and sensitivity, such that increased specificity can come at the cost of sensitivity, or such that increased sensitivity can come at the cost of specificity. Those of skill in the art will appreciate that a useful biomarker need not have 100% specificity and/or 100% accuracy and may reflect a balance of these or other considerations. Those of skill in the art will appreciate that markers may differ in their specificity and/or sensitivity as related to a particular biological event or state of interest. In some instances, a biomarker can be referred to as a “marker.”

Comparable: As used herein, the term “comparable” refers to members within sets of two or more conditions, circumstances, agents, entities, populations, etc., that may not be identical to one another but that are sufficiently similar to permit comparison there between, such that one of skill in the art will appreciate that conclusions can reasonably be drawn based on differences or similarities observed. In some embodiments, comparable sets of conditions, circumstances, agents, entities, populations, etc. are typically characterized by a plurality of substantially identical features and zero, one, or a plurality of differing features. Those of ordinary skill in the art will understand, in context, what degree of identity is required to render members of a set comparable. For example, those of ordinary skill in the art will appreciate that members of sets of conditions, circumstances, agents, entities, populations, etc., are comparable to one another when characterized by a sufficient number and type of substantially identical features to warrant a reasonable conclusion that differences observed can be attributed in whole or part to non-identical features thereof.

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total or free amount of a therapeutic composition or agent administered to a particular subject is determined by one or more attending physicians and may involve administration of multiple dosage forms.

Dosing regimen: As used herein, the term “dosing regimen” can refer to a set of one or more same or different unit doses administered to a subject, typically including a plurality of unit doses administration of each of which is separated from administration of the others by a period of time. In various embodiments, one or more or all unit doses of a dosing regimen may be the same or can vary (e.g., increase over time, decrease over time, or be adjusted in accordance with the subject and/or with a medical practitioner's determination). In various embodiments, one or more or all of the periods of time between each dose may be the same or can vary (e.g., increase over time, decrease over time, or be adjusted in accordance with the subject and/or with a medical practitioner's determination). In some embodiments, a given therapeutic agent has a recommended dosing regimen, which can involve one or more doses. Typically, at least one recommended dosing regimen of a marketed drug is known to those of skill in the art. In some embodiments, a dosing regimen is correlated with a desired or beneficial outcome when administered across a relevant population (i.e., is a therapeutic dosing regimen).

Downstream and Upstream: As used herein, the term “downstream” means that a first DNA region is closer, relative to a second DNA region, to the 3′-terminus of a nucleic acid that includes the first DNA region and the second DNA region. As used herein, the term “upstream” means a first DNA region is closer, relative to a second DNA region, to the 5′-terminus of a nucleic acid that includes the first DNA region and the second DNA region.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be linked to one another in the engineered polynucleotide. Those of skill in the art will appreciate that an “engineered” nucleic acid or amino acid sequence can be a recombinant nucleic acid or amino acid sequence. In some embodiments, an engineered polynucleotide includes a coding sequence and/or a regulatory sequence that is found in nature operably linked with a first sequence but is not found in nature operably linked with a second sequence, which is in the engineered polynucleotide and operably linked in with the second sequence by the hand of man. In some embodiments, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

Expression: As used herein, “expression” refers individually and/or cumulatively to one or more biological process that result in production from a nucleic acid sequence of an encoded agent, such as a polypeptide. Expression specifically includes either or both of transcription and translation.

Gene or Transgene: As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Host cell/target cell: As used herein, “host cell” refers to a cell into which exogenous nucleic acid (recombinant or otherwise), such as a transgene, has been introduced. Those of skill in the art appreciate that a “host cell” can be the cell into which the exogenous nucleic acid was initially introduced and/or progeny or copies, perfect or imperfect, thereof. In some embodiments, a host cell includes one or more viral genes or transgenes. In some embodiments, an intended or potential host cell can be referred to as a “target cell.”

Identity. As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. Calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. The comparison of sequences and determination of percent identity between two sequences can be accomplished using a computational algorithm, such as BLAST (basic local alignment search tool).

Improve, increase, inhibit and reduce: As used herein, the terms “improve”, “increase”, “inhibit”, and “reduce”, and grammatical equivalents thereof, indicate qualitative or quantitative difference from a reference.

Inhibitory agent: As used herein, the term “inhibitory agent” refers to an entity, condition, or event whose presence, level, or degree correlates with decreased level, expression, or activity of a target. In some embodiments, an inhibitory agent may be act directly (in which case it exerts its influence directly upon its target, for example by binding to the target); in some embodiments, an inhibitory agent may act indirectly (in which case it exerts its influence by interacting with and/or otherwise altering a regulator of the target, so that level and/or activity of the target is reduced). In some embodiments, an inhibitory agent is one whose presence or level correlates with a target level or activity that is reduced relative to a particular reference level or activity (e.g., that observed under appropriate reference conditions, such as presence of a known inhibitory agent, or absence of the inhibitory agent in question, etc.).

Isolated: As used herein, “isolated” refers to a substance and/or entity that has been (a) separated from at least some of the components with which it was associated when initially produced (whether in nature and/or in an experimental setting), and/or (b) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, (i) by virtue of its origin or source of derivation is not associated with some or all of the components that accompany it in its native state in nature; (ii) it is substantially free of other polypeptides or nucleic acids of the same species from the species that produces it in nature; (iii) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components (i) with which it is associated in nature; and/or (ii) with which it was associated when initially produced.

Operably linked: As used herein, “operably linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to a composition in which an active agent is formulated together with one or more pharmaceutically acceptable carriers.

Polypeptide: As used herein, “polypeptide” refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may include natural amino acids, non-natural amino acids, or both. In some embodiments, a polypeptide may include only natural amino acids or only non-natural amino acids. In some embodiments, a polypeptide can include D-amino acids, L-amino acids, or both. In some embodiments, a polypeptide may include only L-amino acids. In some embodiments, a polypeptide may include one or more pendant groups or other modifications, e.g., one or more amino acid side chains, e.g., at the polypeptide's N-terminus, at the polypeptide's C-terminus, at non-terminal amino acids, or at any combination thereof. In some embodiments, such pendant groups or modifications may be selected from the group including acetylation, amidation, lipidation, methylation, phosphorylation, glycosylation, glycation, sulfation, mannosylation, nitrosylation, acylation, palmitoylation, prenylation, pegylation, etc., including combinations thereof. In some embodiments, a polypeptide may be cyclic, and/or may include a cyclic portion.

In some embodiments, the term “polypeptide” may be appended to a name of a reference polypeptide, activity, or structure to indicate a class of polypeptides that share a relevant activity or structure. For such classes, the present specification provides and/or those skilled in the art will be aware of exemplary polypeptides within the class whose amino acid sequences and/or functions are known. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that can in some embodiments be or include a characteristic sequence element) that shows very high sequence identity, often greater than 90% or even 95%, 96%, 97%, 98%, or 99%. Such a conserved region usually encompasses at least 3-4 and in some instances up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide can include a fragment of a parent polypeptide. In some embodiments, a useful polypeptide may include a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered to have occurred when onset of a disease, disorder, or condition has been delayed for a period of time that is predefined or understood by those of skill in the art.

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences.

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represents measure under comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof.

Regulatory Sequence: As used herein in the context of expression of a nucleic acid coding sequence, a regulatory sequence is a nucleic acid sequence that controls expression of a coding sequence. In some embodiments, a regulatory sequence can control or impact one or more aspects of gene expression (e.g., cell-type-specific expression, lineage-specific expression, inducible expression, etc.).

Sample: As used herein, the term “sample” typically refers to a sample obtained or derived from a biological source (e.g., a tissue or organism or cell culture) of interest, as described herein. In some embodiments, a biological source is or includes an organism, such as an animal or human. In some embodiments, a sample is or include biological tissue or fluid. In some embodiments, a sample can be or include cells, tissue, or bodily fluid. In some embodiments, a sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or bronchioalveolar lavages, aspirates, scrapings, or bone marrow. In some embodiments, a sample is or includes cells obtained from a single subject or from a plurality of subjects. A sample can be a “primary sample” obtained directly from a biological source, or can be a “processed sample” (e.g., a sample prepared from a primary sample, e.g., by a process such as isolation, e.g., of mRNA, DNA, or protein, by a process that modifies the primary sample's chemical structure, and/or by a process that produces a new or different composition that represents one or more components or properties of the primary sample).

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.”

Therapeutic agent: As used herein, the term “therapeutic agent” refers to any agent that elicits a desired pharmacological effect when administered to a subject. In some embodiments, an agent is considered to be a therapeutic agent if it demonstrates a statistically significant effect across an appropriate population. In some embodiments, the appropriate population can be a population of model organisms or a human population. In some embodiments, an appropriate population can be defined by various criteria, such as a certain age group, gender, genetic background, preexisting clinical conditions, etc. In some embodiments, a therapeutic agent is a substance that can be used for treatment of a disease, disorder, or condition. In some embodiments, a therapeutic agent is an agent that has been or is required to be approved by a government agency before it can be marketed for administration to humans. In some embodiments, a therapeutic agent is an agent for which a medical prescription is required for administration to humans.

Therapeutically effective amount: As used herein, “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that a therapeutically effective amount does not necessarily achieve successful treatment in every particular treated individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition.

Unit dose: As used herein, the term “unit dose” refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose can be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose can be present in a formulation that includes any of a variety of components in addition to the therapeutic agent(s). For example, acceptable carriers (e.g., pharmaceutically acceptable carriers), diluents, stabilizers, buffers, preservatives, etc., can be included. It will be appreciated by those skilled in the art, in many embodiments, a total appropriate daily dosage of a particular therapeutic agent can include a portion, or a plurality, of unit doses, and can be decided, for example, by a medical practitioner within the scope of sound medical judgment. In some embodiments, the specific effective dose level for any particular subject or organism can depend upon a variety of factors including the disorder being treated and the severity of the disorder; activity of specific therapeutic agent(s) employed; specific composition employed; age, body weight, general health, sex and diet of the subject; time of administration, and rate of excretion of the specific therapeutic agent(s) employed; duration of the treatment; drugs and/or additional therapies used in combination or coincidental with specific therapeutic agent(s) employed, and like factors well known in the medical arts.

### BRIEF DESCRIPTION OF THE DRAWINGS

FIG. 1 is a schematic representation of an exemplary helper dependent adenoviral (HDAd) support vector (top) and an exemplary HDAd viral gene therapy vector (bottom). The support vector encodes (i) a Flpe recombinase operably linked to an EF1α promoter and (ii) a transposase (SB100x) operably linked to a PGK promoter, positioned between adenoviral inverted terminal repeats (ITRs). A stuffer is also included in the support vector to produce a viral vector genome of a size efficiently packaged by the adenovirus. The viral gene therapy vector includes a therapeutic payload that includes a nucleic acid sequence encoding a therapeutic protein (rh γ-globin) that is operably linked to a both a β-globin promoter and a β-globin locus control region (LCR), and that is further operably linked to 3′ UTR and chicken hypersensitive site 4 (cHS4; the chicken p-like globin gene cluster) regulatory regions. The viral gene therapy vector further (optionally) includes a nucleic acid sequence encoding a MGMT^{P140K} selectable marker operably linked to a PGK promoter and to a polyA sequence (PA). The therapeutic payload is flanked by inverted repeats (IRs) that are SB100x targets for transposition, whereby the therapeutic payload can be integrated into a host cell genome. The IRs are in turn flanked by frt sites that, upon exposure to Flpe, circularize the flanked nucleic acid to facilitate transposition of the therapeutic payload. The viral gene therapy vector further includes adenoviral ITRs.

FIG. 2 is a schematic representation of an exemplary method of viral gene therapy that includes a viral gene therapy vector used in combination with an immune suppression regimen of the present disclosure. In this schematic, days are identified with reference to the last day on which viral vector is administered to the subject.

FIG. 3 is a schematic representation of an exemplary selection regimen for viral gene therapy including a viral gene therapy vector that includes an MGMT^{P140K} selectable marker, such that selection with O⁶BG/BCNU selects for cells that express and/or have integrated the selectable marker.

FIG. 4 is a schematic representation of an exemplary stem cell mobilization regimen. In this schematic, days are identified with reference to the last day on which viral vector is administered to the subject.

FIG. 5 is a schematic representation of an exemplary method of viral gene therapy that includes a viral gene therapy vector in combination with an immune suppression regimen, a selection regimen, and a stem cell mobilization regimen.

FIG. 6 is a schematic representation of an exemplary helper dependent adenoviral (HDAd) support vector (top) and an exemplary HDAd viral gene therapy vector (bottom). The support vector encodes (i) a Flpe recombinase operably linked to an EF1α promoter and (ii) a transposase (SB100x) operably linked to a PGK promoter, positioned between adenoviral inverted terminal repeats (ITRs). A stuffer is also included in the support vector to produce a viral vector genome of a size efficiently packaged by the adenovirus. The viral gene therapy vector includes a therapeutic payload that includes a nucleic acid sequence encoding a therapeutic protein (rh γ-globin) that is operably linked to a both a β-globin promoter and a β-globin locus control region (LCR), and that is further operably linked to 3′ UTR and chicken hypersensitive site 4 (cHS4; the chicken β-like globin gene cluster) regulatory regions. The viral gene therapy vector further (optionally) includes a nucleic acid sequence encoding a MGMT^{P140K} selectable marker operably linked to an EF1α promoter. The therapeutic payload is flanked by inverted repeats (IRs) that are SB100x targets for transposition, whereby the therapeutic payload can be integrated into a host cell genome. The IRs are in turn flanked by frt sites that, upon exposure to Flpe, circularize the flanked nucleic acid to facilitate transposition of the therapeutic payload. The viral gene therapy vector further includes a separate cassette with promoters and nucleic acid sequences encoding a base editing CRISPR system. The viral gene therapy vector further includes adenoviral ITRs.

FIGS. 7A-7D. In vivo HSC gene therapy. FIG. 7A illustrates in vivo transduction in representative embodiments avoids HSC collection and ex vivo manipulation; requires no myeloablation, conditioning, or transplantation; and provides low-cost vector manufacturing. FIG. 7B is a graph showing percentage of GFP+ cells in PMBC in six animals at the times indicated over the 26 week study; red arrows indicate O⁶BG/BCNU administration (dosing schedule as illustrated in FIG. 7D). FIG. 7C shows an exemplary HDAd-mgmt/GFP/HDAd-SB vector system; mgmt^{P140K} provides a mechanism for drug resistance and the selective expansion of gene-modified cells. FIG. 7D is a representative dosing schedule for use with in vivo HSC gene therapy. AMD3100=plerixafor.

FIGS. 8A-8D. FIG. 8A is a schematic of exemplary vectors. (FIG. 8B) Vector-design for NHP #1: HDAd5/35++ combination. Validated in CD46tg and Townes models: additive HbF expression (>20%). The shown Ad5/35 “combination” donor vector uses CRISPR/Cas9 to target BCL11aE and HBG1/2 BCL11a repressor protein binding sites and SB100x for integration of a transposon encoding hu-γ-globin and MGMT^{P140K}. (FIG. 8C) Vector-design for NHP #2: The shown donor vector includes (i) CRISPR/Cas9 for targeted insertion of a payload including γ-globin and an MGMT^{P140K} selectable marker into an AAVS1 locus; and (ii) CRISPR/Cas9 to target HBG1/2 BCL11a repressor protein binding sites and BCL11aE. (FIG. 8D) Vector-design for NHP #3: HDAd-rh-combination. The shown Ad5/35 “combination” donor vector includes (i) CRISPR/Cas9 to target HBG1/2 BCL11a repressor protein binding sites; and (ii) SB100x for integration of a payload encoding rh-γ-globin and an MGMT^{P140K} selectable marker Shown donor vectors permit in vivo selection of transduced cells expressing mgmt^{P140k}, LCR β-globin promoter driven exogenous γ-globin, and reactivation of endogenous γ-globin via CRISPR/Cas9-mediated disruption of repressor binding region of γ-globin promoter.

FIGS. 9A-9D. HDAd5/35++ vectors preferentially transduce HSCs (through CD46).

FIGS. 10A-10D. Efficient HSC mobilization in rhesus macaques by G-CSF/AMD3100. HDAd injection into mobilized rhesus. (FIG. 10A) Timing of GCSF, AMD3100 and HDAd injection. FIGS. 10B-10D Numbers of primitive (CD34+/CD45RA−/CD90+) HSCs in peripheral blood. HDAd was injected at the two peaks of mobilization. (FIG. 10B) Vector dosing in NHP #1 was conservative (0.5 to 1.65×10¹² vp/kg). (FIG. 100) HDAd doses for NHP #2 were both 1.6×10¹² vp/kg. (FIG. 10D) HDAd doses in NHP #3, showing that the second dose of HDAd was reduced, although this reduction was based on a neutrophil count later found to be erroneous.

FIGS. 11A-11C. FIG. 11A illustrates innate immune response after intravenous adenoviral vector injection and pharmacological interventions. FIG. 11B is a dosing schedule; and FIG. 11C is a graph, illustrating that dexamethasone treatment in mice leads to blunted cytokine response.

FIGS. 12A-12E. Cytokine prophylaxis: Rhesus serum cytokine levels were measured using a cytometric bead array. Only IL-6 (FIGS. 12A-12C) and TNFα (FIGS. 12D, 12E) were detectable. Addition of Tocilizumab (anti-IL6R) and Anakinra (ID R-antagonist) further decreases serum IL-6 and TNFα. (FIGS. 12A-12C) Serum IL-6. (FIGS. 12D, 12E) Serum TNFα. TNFα was not detectable in NHP #2 (+Toci, +Anakinra)

FIGS. 13A-13D. There is no liver toxicity after intravenous HDAd5/35++ injection in (FIGS. 13A, 13B) mice or (FIGS. 13C, 13D) rhesus macaques. Shows little or no transduction of the liver with HDAd5/35++ in mice and no elevation in the serum levels of the liver enzymes AST and ALT in mice and NHPs. This is an important toxicity readout and shows that HDAd5/35++ has little liver toxicity. In HDAd5/35++, short Ad35 fiber shaft prevents factor X binding to hexon and thus hepatocyte transduction.

FIGS. 14A-14C. 5% of primitive HSCs (CFU) were stably transduced before in vivo selection (NHP #3). FIG. 14A outlines the protocol for the CFU assay. Primitive HSCs are transduced by HDAd5/35++ (all episomal at early times) with 5% of primitive HSCs transduced (stable integration) just prior to selection at week four, which increases to 10% at week 8 after treatment with a single round of O⁶BG and BCNU in rhesus macaques (NHP #3).

FIGS. 15A-15C. After in vivo selection: 90% γ-globin marking in peripheral red blood cells (NHP #1). (FIG. 15A, 15B) Flow cytometry for γ-globin (rhesus+human) on peripheral RBCs (FIG. 15A) and erythroid progenitors in bone marrow aspirates (BMA) (FIG. 15B). Downward arrows indicate O⁶BG/BCNU treatment. FIG. 15C Shows 100% marking of rhesus RBCs with human gamma globin following three rounds of O⁶BG and BCNU (NHP #1). These results demonstrate that the selection protocol is efficacious.

FIGS. 16A-16I. Antibody responses against the HDAd5/35++ vector and transgene products. Serum antibodies against HDAd5/35++ for NHP #1 (FIG. 16A) and NHP #3 (FIG. 16B). mRNA levels of the genome-editing enzymes (FIG. 16G SB100x; FIG. 16H Flpe; FIG. 16I Cas9) and serum antibodies (FIG. 16D anti-Flpe antibodies; FIG. 16E anti-SB100x antibodies; and FIG. 16F anti-Cas9 serum antibodies) against these enzymes, as well as against GFP+ (FIG. 16C) (NHP #1). Both the expression and immune response kinetics was similar in NHP #3 (not shown). These data show typical well-known antibody responses to viral gene therapy capsids and the exogenous GFP reporter encoded by the transgene. In contrast, little or no antibody responses were seen against Flpe, SB, or Cas9 further supporting the safety and long term transgene expression in this system.

FIGS. 17A-17F. Immune response against transgene products result in loss of transduced cells (NHP #3). γ-globin expression was analyzed by flow cytometry. (FIG. 17A) RBCs. (FIG. 17B) bone marrow aspirates. FIG. 17C shows representative plots. Arrows indicate O⁶BG/BCNU treatment. (FIGS. 17E, 17F) Higher transgene expression levels were present in NHP #3. These data show a loss of human gamma globin expressing rhesus RBCs coupled with a loss of human mGmT^{P140K} expression in PBMCs that appears to be due to antibody responses to human MGMT^{P140K} in the rhesus model. This is probably due to the high levels of gamma globin and MGMT expression in NHP #3 compared with NHP #1. The loss of gamma globin and MGMT expression was not seen in NHP #1.

FIGS. 18A-18C. Serum antibody (IgG and IgM) titers against recombinant human MGMT protein in NHP #3 (FIG. 18A) and NHP #1 (FIG. 18B). (FIG. 18C) Next vector: rhesus γ-globin and mgmt^{P140K}. In NHP #1, the anti-MGMT response was blunted by continuous exposure to immunosuppressive agents during the course of the study. Anti-MGMT antibodies increased in NHP #3 when tacrolimus was stopped.

FIGS. 19A-19C. Vector clearance from blood. Vector genomes in serum samples were measured by qPCR.

FIGS. 20A-20B. Weight and hematological data. For the blood data, the normal range is shown in transparent grey.

FIGS. 21A-21C. Cellular bone marrow composition. (FIGS. 21A, 21B): Percentage of lineage+ cells (CD20, CD3, CD14) and HSCs (CD34). CD34 cells (NHP #3) are separately shown in FIG. 21C.

FIG. 22. Vector copy number per cell (measured by qPCR with human mgmtP140K primers). Tissues were harvested at day 3 after the last HDAd injection. Gall bladder, jejunum, lungs, and liver contained large amounts of blood.

FIGS. 23A-23C. Vector copy numbers in PBMCs, in NHP #1 (FIG. 23A), NHP #2 (FIG. 23B), and NHP #3 (FIG. 23C).

FIGS. 24A-24D. Preferential transduction of CD34+ cells. Shown are VCN/cell from total bone marrow mononuclear cells (MNCs) and bone marrow CD34+ cells at day 3 and 8 after HDAd injection. The right panel shows mgmt^{P140K} mRNA levels. (The mgmt gene is under the control of the ubiquitously active EF1α promoter).

FIGS. 25A, 25B. Percentage of vector positive progenitor colonies. CD34+ cells were plated for progenitor colony assays. 12 days after plating, about 100 single colonies were harvested and genomic DNA was analyzed by qPCR using mgmt^{P140K}-specific primers.

FIGS. 26A-26C. Human γ-globin and mgmt^{P140K} transgene expression. (FIG. 26A) Human γ-globin levels measured by HPLC. HPLC can distinguish between rhesus and human γ-globin chains. (FIG. 26B) Representative HPLC. (FIG. 26C.) Human mgmt^{P140K} mRNA expression measured by qRT-PCR. Note the similar kinetics.

FIGS. 27A, 27B. Expression of rhesus γ-globin and CRISPR cleavage of target site.

FIGS. 28A-28C. Potential sequestration of HDAd5/35++ by CD46 present on rhesus erythrocytes. (FIG. 28A) The flow cytometry results support CD46tg mice and rhesus monkey as suitable model to evaluate for the transduction of Ad35-based vectors. (FIG. 28B) To test this hypothesis, an in vitro transduction study was performed with Ad5/35++ GFP vector and the addition of whole blood from different animal species and model. In brief, whole blood was collected in EDTA from human, rhesus monkey, CD46tg mice and 057Bl/6 mice. The EDTA whole blood was then washed with PBS, pelleted and resuspended in cell culture medium (Dulbecco's Modified Eagle Medium [DMEM]/Fetal Calf Serum [FCS]). The whole blood was added to HEK293 cells and the cells were transduced with Ad5/35++ GFP vector at the following MOI: 0, 10, 100, 250, 500 and 1000. After 1 hr, the 293 cells were washed with PBS and added with fresh medium. Vector transduction efficiency was analyzed for GFP positive cells by flow cytometry at 24 hrs. (FIG. 28C) Soluble/shed CD46 levels in serum after IV HDAd5/35++ injection in NHP #2 and NHP #3.

FIG. 29. Rhesus serum cytokine levels were measured using a cytometric bead array. Serum IL-6 (pg/mL) levels are shown for NHP #4. Relative dates, and times, are indicated along the X axis. Arrows indicate administration of adenoviral vector.

FIG. 30. Rhesus serum cytokine levels were measured using a cytometric bead array. TNFα (pg/mL) levels are shown for NHP #4. Relative dates, and times, are indicated along the X axis. Arrows indicate administration of adenoviral vector.

FIG. 31. Rhesus serum cytokine levels were measured using a cytometric bead array. Serum IL-6 (pg/mL) levels are shown for NHP #5. Relative dates, and times, are indicated along the X axis. Arrows indicate administration of adenoviral vector.

FIG. 32. Rhesus serum cytokine levels were measured using a cytometric bead array. TNFα (pg/mL) levels are shown for NHP #5. Relative dates, and times, are indicated along the X axis. Arrows indicate administration of adenoviral vector.

### DETAILED DESCRIPTION

Challenges of in vivo Therapy. Viral gene therapy can induce counterproductive immune responses in mammals. For instance, viral vectors can invoke innate immune responses through any of a number of pathways, including through detection of pathogen-associated molecular patterns. Innate immune sensors that detect viral vectors can be found in the cytoplasm, endosome, or surface of cells and recognize viral features such as the capsid, envelope, viral DNA, or viral RNA. Typical innate immune responses include a cytokine response induced within an hour of administration of a viral gene therapy vector to a subject. Antiviral cytokines can be produced by cells such as antigen-presenting cells (APCs), including without limitation plasmacytoid dendritic cells (DCs), conventional DCs, macrophages, and B-cells. Innate immune responses can include proinflammatory effects that recruit effector lymphocytes, inhibit transduction of target cells by the gene therapy vector, and facilitate counterproductive adaptive immune system activity.

Adaptive immunity can also prove problematic where expression of an exogenous coding nucleic acid sequence produces a product that is a non-native antigen in a subject, or an antigen that is otherwise recognized as foreign by the adaptive immune system. In such instance, the adaptive immune system can mediate destruction of subject cells expressing an exogenous coding nucleic acid sequence.

Some adenoviral vectors have been shown to induce both innate immune responses and adaptive immune responses. Adenoviral vectors can induce innate immune responses through various pathways including complement activation. Adenoviral vectors can rapidly induce production of proinflammatory cytokines, e.g., within 1 hour of administration of an adenoviral gene therapy vector. For certain common adenoviruses, many humans have pre-existing neutralizing antibodies prior to administration of an adenoviral gene therapy vector.

The present disclosure provides, among other things, immune suppression regimens that reduce immunotoxicity resulting from administration of a viral vector and/or from gene therapy, e.g., in vivo gene therapy using a viral gene therapy vector. The present disclosure provides, among other things, immune suppression regimens that include an inflammatory signal inhibitor, optionally wherein the inflammatory signal is an interleukin-1 (IL-1) signal inhibitor such as an IL-1 receptor (IL-1R) signal inhibitor.

Immune Suppression Agents. The present disclosure includes immune suppression regimens that reduce the immunotoxicity of gene therapy, e.g., in vivo gene therapy that includes administering a viral gene therapy vector. Immune suppression regimens of the present disclosure can include one or more immune suppression agents including inflammatory signal inhibitors. Immune suppression regimens of the present disclosure can include one or more immune suppression agents including any of one or more of:

(i) an inflammatory signal inhibitor, such as an interleukin-1 (IL-1) signal inhibitor;

(ii) an IL-6 signal inhibitor;

(iii) a corticosteroid;

(iv) a calcineurin inhibitor;

(v) a TNF-α signal inhibitor;

(vi) a JAK signal inhibitor; and

(vii) an inhibitor of co-stimulatory signaling in T cell activation.

Certain immune suppression regimens of the present disclosure can include one or more immune suppression agents including any of one or more of:

(i) an interleukin-1 (IL-1) signal inhibitor;

(ii) an IL-6 signal inhibitor;

(iii) a corticosteroid;

(iv) a calcineurin inhibitor, and

(vii) an inhibitor of co-stimulatory signaling in T cell activation.

In various embodiments, an immune suppression regimen that reduces immunotoxicity of gene therapy, e.g., in vivo gene therapy, is administered to a subject in conjunction with a viral gene therapy regimen including one or more viral vector agents selected from:

(i) a viral gene therapy vector; and

(ii) a support vector.

In various embodiments, any of the immune suppression agents can be administered to a subject in a single dose or in a plurality of doses. In various embodiments, any of the immune suppression agents can be administered to a subject on a single day or on a plurality of days. In various embodiments, any of the immune suppression agents can be administered at a daily dose that is administered to the subject in a single dose or in a plurality of separate doses. In various embodiments, a dose of any of the immune suppression agents can be administered in a single unit dose that includes the entire dose and/or entire daily dose or in a plurality of unit doses that together provide the entire dose and/or entire daily dose.

In various embodiments, a dosage form can include an amount of each of one or more agents that are immune suppression agents. In various embodiments, a dosage form can include an amount of each of two or more agents that are immune suppression agents that are of the same immune suppression agent class or a plurality of immune suppression agent classes. In various embodiments, a dosage form can include at least one immune suppression agent of a first immune suppression agent class and at least one immune suppression agent of a second immune suppression agent class that is a different immune suppression agent class than the first immune suppression agent class.

Inflammatory Signal Inhibitors. A wide variety of signals have been identified that can contribute to inflammatory responses, e.g., following administration to a subject of an exogenous agent such as a viral gene therapy vector. The pathways that transduce inflammatory signals typically include, at least in part, pro-inflammatory signaling agents and pro-inflammatory signaling receptors for which the pro-inflammatory signaling agents act as ligands. In various instances, binding of a pro-inflammatory signaling receptor and a pro-inflammatory signaling receptor mediate an immunological and/or inflammatory response.

Examples of pro-inflammatory signaling agents include cytokines IL-Iβ, IL-1α, IL-6, TNF-α, TGF-β, IFN-γ, IL-8 (also referred to in the art as CXCL8), IL-12, GM-CSF, IL-15, and CCL2.

Examples of pro-inflammatory signaling receptors (and their ligands) include IL-1R (IL-1β, IL-1α), IL-3R, IL-4Ra, IL-5R, IL-6Ra (IL-6), IL-36R, TNFR1 (TNF-α), TGFβR1/TGFβR2 (TGF-β), IFNGR (IFN-γ), interferon-α/β receptor, IL-8R (including IL-8RA and IL-8RB forms, also referred to as CXCR1 and CXCR2 forms) (IL-8/CXCL8), IL-12R (IL-12), GM-CSFB (GM-CSF), IL-15R (IL-15), CCR2 (CCL2), and CCR4 (CCL2).

In various embodiments, an inflammatory signal inhibitor can be an agent that binds or modifies a pro-inflammatory signaling agent such that the pro-inflammatory signaling agent cannot bind a pro-inflammatory signaling receptor. In various embodiments, an inflammatory signal inhibitor can be an agent that binds or modifies a pro-inflammatory signaling agent such that the pro-inflammatory signaling agent binds a pro-inflammatory signaling receptor with reduced affinity, avidity, or frequency, e.g., as compared to a reference pro-inflammatory signaling agent not exposed to the inhibitor, including without limitation a blocking agent. In various embodiments, an inflammatory signal inhibitor can be an agent that binds or modifies a pro-inflammatory signaling agent such that the pro-inflammatory signaling agent has a decreased half-life, e.g., as compared to a reference pro-inflammatory signaling agent not exposed to the inhibitor. In various embodiments, an agent that is an inhibitor of a pro-inflammatory signaling agent can be referred to as an antagonist of the pro-inflammatory signaling agent. Accordingly, for example, an inhibitor of a receptor can be referred to as a receptor antagonist (e.g., anakinra is an exemplary IL-1 receptor antagonist).

In various embodiments, an inflammatory signal inhibitor can be an agent that binds or modifies a pro-inflammatory signaling receptor such that the pro-inflammatory signaling receptor cannot bind a pro-inflammatory signaling agent. In various embodiments, an inflammatory signal inhibitor can be an agent that binds or modifies a pro-inflammatory signaling receptor such that the pro-inflammatory signaling receptor binds a pro-inflammatory signaling agent with reduced affinity, avidity, or frequency, e.g., as compared to a reference pro-inflammatory signaling receptor not exposed to the inhibitor, including without limitation a blocking agent. In various embodiments, an inflammatory signal inhibitor can be an agent that binds or modifies a pro-inflammatory signaling receptor such that the pro-inflammatory signaling receptor has a decreased half-life, e.g., as compared to a reference pro-inflammatory signaling receptor not exposed to the inhibitor. In various embodiments, an agent that is an inhibitor of a pro-inflammatory signaling receptor can be referred to as an antagonist of the pro-inflammatory signaling receptor.

In various embodiments, delivery of an inflammatory signal inhibitor to a subject causes reduced phosphorylation of a pro-inflammatory signaling receptor in the subject, where phosphorylation of the pro-inflammatory signaling receptor causes inflammation or is positively associated with inflammation, as compared to a reference not exposed to the inflammatory signal inhibitor. In various embodiments, delivery of an inflammatory signal inhibitor to a subject causes reduced de-phosphorylation of a pro-inflammatory signaling receptor in the subject, where de-phosphorylation of the pro-inflammatory signaling receptor causes inflammation or is positively associated with inflammation, as compared to a reference not exposed to the inflammatory signal inhibitor.

In various embodiments, delivery of an inflammatory signal inhibitor to a subject causes reduced inflammation, as compared to a reference not exposed to the inflammatory signal inhibitor. In various embodiments, delivery of an inflammatory signal inhibitor to a subject causes a reduction in a biomarker indicative of inflammation, as compared to a reference not exposed to the inflammatory signal inhibitor. In various embodiments, a biomarker indicative of inflammation can be a cytokine indicative of immune activation and/or any of one or more of IL-Iβ, IL-2, IL-3, IL-4, IL-5, IL-6, IL-8, IL-10, IL-12, IL-13, IL-15, IL-17, IL-23, IL-27, IL-30, IL-36 IL-1Ra, IL-2R, IFN-α, IFN-b, IFN-γ, MIP-Ia, MIP-Iβ, MCP-1, TNF-α, TNF-β, GM-CSF, G-CSF, CXCL9, CXCL10, VEGF, RANTES, EGF, HGF, FGF-β, CD40, and CD40L. Other exemplary biomarkers can include a measure of the concentration or amount of antibodies to an agent administered to a subject, such as neutralizing antibodies to a vector administered to a subject in an in vivo gene therapy regiment.

In some embodiments, an inflammatory signal inhibitor can be a protein, e.g., a protein that binds a pro-inflammatory signaling agent or a pro-inflammatory signaling receptor. In various embodiments, an inflammatory signal inhibitor can be an antibody, e.g., an antibody or antibody fragment that binds a pro-inflammatory signaling agent or a pro-inflammatory signaling receptor. In various embodiments, an inflammatory signal inhibitor can be a molecule that is not proteinaceous, such as a small molecule inhibitor of a pro-inflammatory signaling agent or a pro-inflammatory signaling receptor.

To provide several non-limiting examples of inflammatory signal inhibitors, in various embodiments, an inflammatory signal inhibitor can be an anti-IL-8/CDCL8 antibody or an anti-CCL2 antibody.

In some embodiments, an inflammatory signal inhibitor is an inhibitor of inosine-5′-monophosphate dehydrogenase, e.g., mycophenolic acid (MPA). An exemplary inflammatory signal inhibitor that delivers MPA to a subject is mycophenolate mofetil (MMF), which is a prodrug of MPA.

Those of skill in the art will appreciate that inflammatory signal inhibitors can include one or more other classes of agents provided in the present disclosure, unless otherwise specified. Those of skill in the art will appreciate that, as used herein, reduction of inflammatory signaling is understood to include, encompass, imply, and/or be interchangeable with a reduction or treatment of inflammation, e.g., a clinically relevant reduction or treatment of inflammation in a subject.

Methods of measuring inflammation in a subject are known to those in the art. For instance, various biomarkers can be used to quantitatively measure, qualitatively measure, quantitatively compare, or qualitatively compare inflammation in or between samples, subjects, or states. Exemplary biomarkers include, without limitation, any of one or more of C-reactive protein (hs-CRP), IL-Iβ, IL-2, IL-3, IL-4, IL-5, IL-6, IL-8, IL-10, IL-12, IL-13, IL-15, IL-17, IL-23, IL-27, IL-30, IL-36, IL-1Ra, IL-2R, IFN-α, IFN-b, IFN-γ, MIP-Ia, MIP-Iβ, MCP-1, TNF-α, TNF-β, GM-CSF, G-CSF, CXCL9, CXCL10, VEGF, RANTES, EGF, HGF, FGF-β, CD40, and CD40L. Other exemplary biomarkers can include a measure of the concentration or amount of antibodies to an agent administered to a subject, such as neutralizing antibodies to a vector administered to a subject in an in vivo gene therapy regiment.

IL-1 Signal Inhibitors. In various embodiments, an inflammatory signal inhibitor can be an agent that is an inhibitor of the pro-inflammatory signaling agent IL-1β and/or IL-1α, or of the pro-inflammatory signaling receptor IL-1R, where such inflammatory signal inhibitors can be cumulatively referred to as IL-1 signal inhibitors. In various embodiments, an IL-1 signal inhibitor can be an agent that competitively inhibits binding of IL-1β and/or IL-1a with IL-1R. For example, canakinumab (ACZ885) is a human anti-IL-1β monoclonal antibody that inhibits binding of IL-1β with IL-1R, and thus reduces inflammatory signaling.

Another example of an inflammatory signal inhibitor is the IL-1 signal inhibitor rilonacept. Rilonacept is a soluble agent that includes the ligand-binding domains of (i) the extracellular portions of the human IL-1 receptor (IL-1R1) and (ii) the IL-1 receptor accessory protein (IL-1RAcP), which ligand-binding domains are linked to the Fc region of human IgG1. Rilonacept can act as a decoy receptor and/or antagonize IL-1 activation.

Another example of an inflammatory signal inhibitor is an IL-1 signal inhibitor that is an IL-1 receptor (IL-1R) agent engineered such that it binds IL-1β and/or IL-1a but does not transduce a pro-inflammatory signal. Engineered IL-1R agents that bind IL-1β and/or IL-1α but do not transduce a pro-inflammatory signal can be referred to as IL-1R antagonists. Another example of an inflammatory signal inhibitor is an IL-1 signal inhibitor that is an IL-1Ra agent engineered such that it binds IL-1R and blocks binding of IL-1R with IL-1β and/or IL-1α but does not transduce a pro-inflammatory signal. Anakinra is an engineered human IL-1 receptor antagonist (IL-1Ra) agent that inhibits signaling through IL-1R by IL-1β and/or IL-1α in humans by competitively binding IL-1R. Anakinra has an amino acid sequence that is similar to a typical human IL-1Ra amino acid sequence, but which differs from a typical human IL-1Ra amino acid sequence at least in that it includes a methionine residue at its amino terminus, as shown in SEQ ID NO: 1. In addition, anakinra is a recombinant protein typically produced from E. coli by expression of a nucleic acid sequence encoding SEQ ID NO: 1, and is non-glycosylated.

(153 aa; anakinra)
SEQ ID NO: 1
MRPSGRKSSKMQAFRIWDVNQKTFYLRNNQLVAGYLQGPNVNLEEKIDV
VPIEPHALFLGIHGGKMCLSCVKSGDETRLQLEAVNITDLSENRKQDKR
FAFIRSDSGPTTSFESAACPGWFLCTAMEADQPVSLTNMPDEGVMVTKF
YFQEDE

In various embodiments, an inflammatory signal inhibitor of the present disclosure is a molecule having at least 80% sequence identity to SEQ ID NO: 1, e.g., at least 80%, 85%, 90%, 91%, 92%, 93%, 94%, 95%, 96%, 97%, 98%, 99%, or 100% identity to SEQ ID NO: 1. In various embodiments, an inflammatory signal inhibitor of the present disclosure is a molecule having at least 80% sequence identity to amino acids 2-153 of SEQ ID NO: 1, e.g., at least 80%, 85%, 90%, 91%, 92%, 93%, 94%, 95%, 96%, 97%, 98%, 99%, or 100% identity to amino acids 2-153 of SEQ ID NO: 1. In various embodiments, an inflammatory signal inhibitor having at least 80% sequence identity to SEQ ID NO: 1 includes an amino-terminal methionine residue.

IL-6 Signal Inhibitors. In various embodiments, an inflammatory signal inhibitor can be an agent that is an inhibitor of the pro-inflammatory signaling agent IL-6, or of the pro-inflammatory signaling receptor IL-6R, where such inflammatory signal inhibitors can be cumulatively referred to as IL-6 signal inhibitors. In various embodiments, an IL-6 signal inhibitor can be an agent that competitively inhibits binding of IL-6 with IL-6R. Exemplary IL-6 signal inhibitors include bazedoxifene, raloxifene, sarilumab, and tocilizumab.

Bazedoxifene is a small molecule inhibitor of IL-6 signaling that is understood to interfere with formation of signaling-competent IL-6 receptor complexes. IL-6 and IL-6Ra form binary complexes that further complex with GP130, and heterodimerization of two such ternary complexes can transduce signals including pro-inflammatory signals. Bazedoxifene is an inhibitor of IL-6/GP130 interaction and can therefore inhibit IL-6 signal transduction.

Raloxifene is a small molecule inhibitor of IL-6 signaling that is understood to interfere with formation of signaling-competent IL-6 receptor complexes. Raloxifene is an inhibitor of IL-6/GP130 interaction and can therefore inhibit IL-6 signal transduction.

Sarilumab is a fully human, monoclonal antibody that inhibits the interleukin-6 (IL-6) pathway by binding and blocking the IL-6 receptor. Sarilumab binds to the IL-6 receptor (both soluble and membrane-bound forms; sIL-6R and mIL-6R), and thereby inhibits IL-6-mediated signal transduction.

Tocilizumab is a humanized IgG1 monoclonal antibody that binds IL-6 receptor with high affinity to the 80 kD component of IL-6R. This binding subsequently inhibits dimerization of the IL-6/IL-6R complex with membrane-bound gp130, preventing signaling. Tocilizumab thereby inhibits IL-6-mediated signal transduction.

Corticosteroids. One or more corticosteroids can be included in immune suppression regimens of the present disclosure. Corticosteroids are anti-inflammatory agents that have structural similarity to the hormone cortisol. Cortisone and hydrocortisone can refer to corticosteroid agents naturally produced by the human adrenal cortex, or to synthetically produced analogs thereof. Examples of corticosteroids also include bethamethasone, prednisone, prednisolone, triamcinolone, methylprednisolone, paramethasone, dexamethasone, ethamethasoneb, fludrocortisone, and budesonide. Those of skill in the art will appreciate that these are representative examples of corticosteroids, and that many examples of corticosteroids are well known in the art. In some embodiments, a corticosteroid is a glucocorticoid. In some embodiments, a corticosteroid is a mineralocorticoid. In certain embodiments, a corticosteroid is dexamethasone.

Calcineurin Inhibitors. Inhibitors of the phosphatase calcineurin can suppress immunotoxicity, e.g., by decreasing lymphocyte proliferation. Exemplary calcineurin inhibitors are tacrolimus and cyclosporine (alternatively spelled ciclosporin or cyclosporin). Calcineurin inhibitors have been used as immunosuppressive agents in organ transplantation to treat or reduce allograft rejection. Cyclosporine is a cyclic endecapeptide, whereas tacrolimus is a macrocyclic lactone.

TNF-α Signal Inhibitors. In various embodiments, an inflammatory signal inhibitor can be an agent that is an antagonist of tumor necrosis factor (TNF)-α and/or is an inhibitor of TNF-α signaling. TNF is can participate in inflammatory and immune responses and can bind to TNF receptor 1 (TNFR1) or TNF receptor 2 (TNFR2). Upon binding to at least certain of its receptors, TNF can trigger pathways including the NFkB and MAPK pathways, which can increase production of numerous inflammatory cytokines. Certain TNF-α signal inhibitors directly bind the cytokine TNF and inhibit interaction of TNF with TNF receptors.

TNF-α signal inhibitors include etanercept, infliximab, adalimumab, certolizumab, pegol, and golimumab. Etanercept is a fusion protein of two TNFR2 receptor extracellular domains and the Fc fragment of human IgG1. Etanercept can inhibit binding of TNF-α and/or TNF-β to TNFRs. Infliximab is a chimeric monoclonal antibody that binds soluble and transmembrane forms of TNF-α and inhibits binding of TNF-α to TNFR. Adalimumab and golimumab are fully human monoclonal antibodies against TNF-α and, like infliximab, bind TNF-α and/or inhibit binding of TNF-α to TNFR. Certolizumab is a humanized Fab fragment conjugated to polyethylene glycol (PEG).

JAK Signal Inhibitors. In various embodiments, an inflammatory signal inhibitor can be an agent that is an antagonist of a Janus kinase (JAK) and/or is an inhibitor of JAK signaling. JAKs, including JAK1, JAK2, JAK3, and TYK2, are cytoplasmic tyrosine kinases associated with cytokine functions, including inflammatory functions. JAKs mediate signal transduction, e.g., by autophosphorylation and/or transphosphorylation of molecules such as signal transducers and activators of transcription (STATs).

Over twenty inhibitors that inhibit signaling of one or more JAKs are known in the art. Not all JAK inhibitors antagonize the same subset of JAKs. For instances, without limitation, some JAK signal inhibitors of the present disclosure are JAK1/2 signal inhibitors. Exemplary JAK inhibitors include baricitinib (inhibits JAK1 and JAK2), tofacitinib (inhibits JAK3, JAK1, and to a lesser degree JAK2), ruxolitinib (inhibits JAK1 and JAK2), and filgotinib (inhibits JAK1). Additional JAK signal inhibitors (e.g., JAK1/2 signal inhibitors) are known in the art. At lease certain further JAK signal inhibitors are provided in Fragoulis (2019 Rheumatology 58(Suppl 1): i43-i54), which is incorporated herein by reference with respect to JAK inhibitors.

Inhibitors of co-stimulatory signaling in T cell activation. In various embodiments, an inhibitor of co-stimulatory signaling in T cell activation is Abatacept. Abatacept is a recombinant fusion protein including the extracellular domain of human cytotoxic T-lymphocyte antigen 4 and a fragment of the Fc domain of human IgG1. Abatacept acts at least in part by competing with CD28 for binding to CD80/CD86, modulating the second co-stimulatory signal required for full T-cell activation. Abatacept acts at least in part by preventing CD80/CD86-CD28 co-stimulatory signal for T cell activation.

### Viral Vector Agents.

Viral Gene Therapy Vectors. Viral gene therapy vectors of the present disclosure include virions that include a viral vector genome, which viral vector genome can include an exogenous coding nucleic acid sequence, optionally where the exogenous coding nucleic acid sequence is present in a therapeutic payload. Administration of a viral gene therapy vector to a subject can deliver the viral vector genome of the viral gene therapy vector to the subject, e.g., to one or more cells of the subject. In various embodiments, the viral vector genome or therapeutic payload thereof includes an exogenous coding nucleic acid sequence that is expressed in one or more cells of the subject and/or incorporated into the genome of one or more cells of the subject. In various embodiments, an exogenous coding nucleic acid sequence encodes a protein, such as a protein capable of achieving a desired therapeutic effect in a subject, including treatment of a disease, disorder, or condition of the subject. In various embodiments, an exogenous coding nucleic acid sequence encodes a small interfering RNA, such as a small interfering RNA capable of achieving a desired therapeutic effect in a subject, including treatment of a disease, disorder, or condition of the subject, optionally wherein the therapeutic effect is mediated by inhibition of expression of a protein. In various embodiments, an exogenous coding nucleic acid sequence encodes an miRNA, such as an miRNA capable of achieving a desired therapeutic effect in a subject, including treatment of a disease, disorder, or condition of the subject, optionally wherein the therapeutic effect is mediated by inhibition of expression of a protein. In various embodiments, an exogenous coding nucleic acid sequence encodes a long non-coding RNA, such as a long non-coding RNA capable of achieving a desired therapeutic effect in a subject, including treatment of a disease, disorder, or condition of the subject, optionally wherein the therapeutic effect is mediated by an expression-regulatory chromatin effect. In various embodiments, an exogenous coding nucleic acid sequence encodes a single guide RNA (sgRNA), such as an sgRNA capable of achieving a desired therapeutic effect in a subject, including treatment of a disease, disorder, or condition of the subject, optionally wherein the therapeutic effect is mediated at least in part by an endonuclease activity, e.g., activity of CRISPR/Cas9. In various embodiments, an exogenous coding nucleic acid sequence encodes an enhancer RNA, such as an enhancer RNA capable of achieving a desired therapeutic effect in a subject, including treatment of a disease, disorder, or condition of the subject, optionally wherein the therapeutic effect is mediated by increased expression of a gene.

In various embodiments, the viral vector genome and/or therapeutic payload includes a promoter or other regulatory region, and the promoter or other regulatory region is operably linked with the exogenous coding nucleic acid sequence. In various embodiments, an exogenous coding nucleic acid sequence encodes a CRISPR system, such as a Cas protein (e.g., a Type II or Type V Cas protein including a Cas9, Cas12a, or Cas 14 protein, or a Type VI Cas protein such as Cas13) and guide RNA molecule capable of achieving a desired therapeutic effect in a subject, including treatment of a disease, disorder, or condition of the subject. In various embodiments, the viral vector genome and/or therapeutic payload includes one or more promoters or other regulatory regions, and the promoter(s) or other regulatory region(s) is operably linked with the exogenous coding nucleic acid sequence.

In various embodiments, an exogenous coding nucleic acid sequence or therapeutic payload including the same can encode an agent that causes increased expression of β-globin and/or γ-globin, or a functional replacement thereof, e.g., in hematopoietic stem cells. In various embodiments, an exogenous coding nucleic acid sequence or therapeutic payload including the same can encode an agent that causes increased expression of Factor VIII or a functional replacement thereof (e.g., ET3) in hematopoietic stem cells. In various embodiments, an exogenous coding nucleic acid sequence or therapeutic payload including the same can encode an agent that causes correction of a genetic lesion that causes sickle cell anemia by gene editing, e.g., a CRISPR system, such as a Cas protein (e.g., a Type II or Type V Cas protein including a Cas9 or Cas12a protein) and guide RNA molecule capable of achieving a desired genetic lesion correction. Exemplary applications of viral gene therapy vectors are further disclosed in, e.g., U.S. Provisional Patent Application No. 62/869,907, filed Jul. 2, 2019, which is incorporated herein by reference in its entirety, and particularly with respect to viral gene therapy vectors and applications of viral gene therapy.

The following references provide particular exemplary sequences of functional globin amino acid sequences, nucleic acid sequences, and amino acid sequences encoded by provided nucleic acid sequences. References 1-4 relate to α-type globin sequences and references 4-12 relate to β-type globin sequences (including β and γ globin sequences): (1) GenBank Accession No. Z84721 (Mar. 19, 1997); (2) GenBank Accession No. NM_000517 (Oct. 31, 2000); (3) Hardison et al., J. Mol. Biol. 222(2):233-249, 1991; (4) A Syllabus of Human Hemoglobin Variants (1996), by Titus et al., published by The Sickle Cell Anemia Foundation in Augusta, Ga. (available online at globin.cse.psu.edu); (5) GenBank Accession No. J00179 (Aug. 26, 1993); (6) Tagle et al., Genomics 13(3):741-760, 1992; (7) Grovsfeld et al., Cell 51(6):975-985, 1987; (8) Li et al., Blood 93(7):2208-2216, 1999; (9) Gorman et al., J. Biol. Chem. 275(46):35914-35919, 2000; (10) Slightom et al., Cell 21(3):627-638, 1980; (11) Fritsch et al., Cell 19(4): 959-972, 1980; (12) Marotta et al., J. Biol. Chem. 252(14):5040-5053, 1977. For additional coding and non-coding regions of genes encoding globins see, for example, by Marotta et al., Prog. Nucleic Acid Res. Mol. Biol. 19, 165-175, 1976, Lawn et al., Cell 21 (3), 647-651, 1980, and Sadelain et al., PNAS. 92:6728-6732, 1995. An exemplary amino acid sequence of hemoglobin subunit β is provided, for example, at NCBI Accession No. P68871. An exemplary amino acid sequence for β-globin is provided, for example, at NCBI Accession No. NP_000509. The present disclosure includes variants of globin proteins provided herein, including variants having at least 70%, 75%, 80%, 85%, 90%, 91%, 92%, 93%, 94%, 95%, 96%, 97%, 98%, 99%, or 100% amino acid identity to an amino acid sequence of a globin protein provided herein.

In various embodiments, an exogenous coding nucleic acid sequence or therapeutic payload including the same can encode a protein selected from γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CI ITA, RFXANK, RFXS, RFXAP, RMRP, DKC1, TERT, TINF2, DCLRE1B, and SLC46A1; FANC family genes including FancA, FancB, FancC, FancD1 (BRCA2), FancD2, FancE, FancF, FancG, Fancl, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3); soluble CD40; CTLA; Fas L; antibodies to CD4, CD5, CD7, CD52, etc.; antibodies to IL1, IL2, IL6; an antibody to TCR specifically present on autoreactive T cells; IL4; IL10; IL12; IL13; IL1Ra, sIL1RI, sIL1R11; sTNFRI; sTNFRII; antibodies to TNF; P53, PTPN22, and DRB1*1501/DQB1*0602; globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; and C9ORF72.

In various embodiments, a therapeutic payload including a promoter and/or other regulatory region(s) operably linked to an exogenous coding nucleic acid sequence, and the viral gene therapy delivers the therapeutic payload to a patient such that the exogenous coding nucleic acid sequence is expressed extra-chromosomally. In various embodiments, a therapeutic payload including a promoter and/or other regulatory region(s) operably linked to an exogenous coding nucleic acid sequence, and the viral gene therapy delivers the therapeutic payload to a patient such that the therapeutic payload is integrated into the genome of a target cell.

A variety of vectors for viral gene therapy, including human viral gene therapy, are known in the art. Exemplary vectors include adenoviruses (Ad), adeno-associated viruses (AAV), herpes simplex viruses (e.g., HSV, HSV1), retroviruses (e.g., MLV, MMSV, MSCV), lentiviruses (e.g., HIV-1, HIV-2), alphaviruses (e.g., SFV, SIN, VEE, M1), flaviviruses (e.g., Kunjin, West Nile, Dengue virus), rhabdoviruses (e.g., rabies, VSV), measles viruses (e.g., MV-Edm), Newcastle disease virus (NDV), poxviruses, and picornaviruses (e.g., coxsackieviruses).

Adenoviral gene therapy vectors can be of any of a variety of serotypes known in the art. Examples of adenoviral gene therapy vectors include adenoviral vectors that target CD46. Examples of adenoviral gene therapy vectors include Ad5 and Ad35. Adenoviral gene therapy vectors can also be pseudotyped adenoviral vectors, such as Ad5/35. Adenoviral gene therapy vectors can be vectors with enhanced binding to CD46, e.g., an Ad35⁺⁺ or Ad5/35⁺⁺ adenoviral vector. Examples of adenoviral gene therapy vectors are further disclosed in U.S. Application No. 62/869,907, filed Jul. 2, 2019, and International Application No. PCT/US2020/040756, filed Jul. 2, 2020, which are incorporated herein by reference with respect to adenoviral gene therapy vectors.

AAV gene therapy vectors can be of any of a variety of serotypes known in the art. In some instances, the AAV vector is selected from AAV1, AAV2, AAV3, AAV4, AAV5, AAV6, AAV7, AAV8, and AAV9. AAV gene therapy vectors can also be pseudotyped AAV vectors, in that In certain instances, the vector is a pseudotyped vector capable of infecting a human cell, e.g., a pseudotyped vector selected from AAV2/1, AAV2/2, AAV2/5, AAV2/6, AAV2/7, AAV2/8, and AAV2/9.

In various embodiments, a viral gene therapy vector is a helper-dependent (HD) viral gene therapy vector. As is well known in the art, one means of engineering viral vectors suitable for gene therapy based on the genomes of natural virus is to produce replication-deficient viruses. Replication-deficient viruses can infect subjects, but their toxicity is limited by their inability to replicate, rendering them particularly suitable for use in subjects. Although viral vector replication can be undesirable when a viral vector is administered to a subject, replication can be required to generate therapeutically useful amounts of viral vector. One solution is the use of an HD viral gene therapy vector that is only able to replicate in the presence of certain proteins that are not encoded by the genome of the HD viral gene therapy vector or the recipient of the viral vector therapy. Instead, the additional proteins required for replication of the HD viral gene therapy vector are provided by expression from a helper virus, plasmid, or other helper nucleic acid. The region(s) of a viral genome responsible for directing packaging can be referred to as the packaging sequence or signal (ψ) or as the encapsidation sequence (E). Because the helper genome, plasmid, or other helper nucleic acid does not include a packaging signal or includes a conditional packaging signal, the helper is not packaged into virions. However, an HD viral vector genome that does include a functional packaging signal is packaged into the HD viral gene therapy vector. Thus, using the helper, the additional proteins can be provided for production of HD viral gene therapy vector in a first context (e.g., in vitro, e.g., in a cell culture), but are not provided when the HD viral gene therapy vector product is administered to a subject.

Helper dependent adenoviral (HDAd) vectors are exemplary of HD viral gene therapy vectors. In some HDAd vector systems, one viral genome (a helper) encodes all of the proteins required for replication but has a conditional defect in the packaging sequence, making it less likely to be packaged into a virion. A second viral genome includes only viral inverted terminal repeats (ITRs), a therapeutic payload, and a normal packaging sequence, which allows this second viral genome to be selectively packaged into HDAd viral vectors and isolated from the producer cells. HDAd viral vectors can be further purified from helper vectors by physical means. In general, some contamination of helper vectors and/or helper genomes in HDAd viral vectors and HDAd viral vector formulations can occur and can be tolerated.

In some HDAd vector systems, a helper genome utilizes a Cre/loxP system. In certain such HDAd vector systems, the HDAd viral gene therapy vector genome includes 500 bp of noncoding adenoviral DNA that includes the adenoviral ITRs which are required for vector genome replication, and ψ which is the packaging sequence required for encapsidation of the vector genome into the capsid. It has also been observed that the HDAd viral gene therapy vector genome can be most efficiently packaged when it has a total length of about 27.7 kb to about 37 kb, which length can be composed, e.g., of a therapeutic payload and or a “stuffer” sequence. The HDAd viral gene therapy vector genome can be delivered to cells, such as 293 cells that expresses Cre recombinase, optionally where the HDAd viral gene therapy vector genome is delivered to the cells in a non-viral vector form, such as a bacterial plasmid form (e.g., where the HDAd viral gene therapy vector genome is constructed as a bacterial plasmid (pHDAd) and is liberated by restriction enzyme digestion). The same cells can be transduced with the helper genome, which can include an E1-deleted, adenoviral vector bearing a packaging sequence flanked by loxP sites so that following infection of 293 cells expressing Cre recombinase, the packaging sequence is excised from the helper genome by Cre-mediated site-specific recombination between the loxP sites. Thus, the HDAd viral gene therapy vector genome can be transfected into 293 cells that express Cre and are transduced or transfected with a helper genome or vector bearing a packaging signal (ψ) flanked by loxP sites such that Cre-mediated excision of ψ renders the helper virus genome unpackageable, but still able to provide all of the necessary trans-acting factors for propagation of the HDAd. After excision of the packaging sequence, a helper genome is unpackageable but still able to undergo DNA replication and thus trans-complement the replication and encapsidation of the HDAd viral gene therapy vector genome. In some embodiments, to prevent generation of replication competent Ad (RCA; E1⁺) as a consequence of homologous recombination between the helper and HDAd viral gene therapy vector genomes present in 293 cells a “stuffer” sequence can be inserted into the E3 region to render any E1⁺ recombinants too large to be packaged. Similar HDAd production systems have been developed using FLP (e.g., FLPe)/frt site-specific recombination, where FLP-mediated recombination between frt sites flanking the packaging signal of the helper genome selects against encapsidation of helper genomes in 293 cells that express FLP (e.g., FLPe). Alternative strategies to select against the helper vectors have been developed.

Viral Gene Therapy Selectable Markers. In various embodiments, a viral gene therapy vector includes a viral gene therapy vector genome that includes a selectable marker, e.g., in a therapeutic payload. Use of a selectable marker in combination with viral gene therapy permits selection of host cells that have been transduced with the viral gene therapy vector and/or that express at least a selectable marker encoded by the genome of the gene therapy vector and/or that have integrated into the genome of the host cell a therapeutic payload of the genome of the gene therapy vector, wherein the therapeutic payload includes a nucleic acid encoding the selectable marker.

In various embodiments, a viral gene therapy vector genome includes a selectable marker that is suitable for in vivo selection in a subject. Selection can increase the population of host cells in a subject to, e.g., at least 40%, 50%, 60%, 70%, 80%, 90%, 95%, or 100% of a target cell population. Inclusion of drug resistance genes as in vivo selectable markers can increase engraftment of genetically modified HSCs following exposure to drugs that are toxic to unmodified cells in the graft. Such in vivo selectable markers include the genes for multi-drug resistance (MDR-1), dihydrofolate reductase (DHFR), and O⁶-methylguanine-DNA methyltransferase (MGMT). To provide just one example, viral vector gene therapy with viral gene therapy vectors including an MGMT^{P140K} selectable marker demonstrate an increase in marked host cells after administration of O⁶-benzylguanine (O⁶BG) and 1,3-bis(2-chloroethyl)-1-nitroso-urea (BCNU) (O⁶BG/BCNU) or tremozolomide.

In various embodiments, a selecting agent, such as O⁶BG in combination with a viral gene therapy vector that includes an MGMT^{P140K} selectable marker, can be administered to a subject, e.g., after administration to the subject of the viral gene therapy vector. In various embodiments, the selecting agent can be administered to a subject at any of one or more of about 1, 2, 3, 4, 5, 6, 7, 8, 9, 10, 11, 12, 13, 14, or 15 weeks after administration of the viral gene therapy vector to the subject, e.g., after administration of a first dose of the viral gene therapy vector to the subject or after administration of a last dose of the viral gene therapy vector to the subject.

In various embodiments, a selecting agent is administered to a subject if marking (transduction) of a target cell population, such as hematopoietic stem cells, is less than 10%, 20%, 30%, 40%, 50%, 60%, 70%, 75%, 80%, 85%, 90%, 95%, or 100%. In various embodiments, a selecting agent is not administered to a subject, and/or administration of a selecting agent is discontinued, if marking of a target cell population, such as hematopoietic stem cells, is more than 10%, 20%, 30%, 40%, 50%, 60%, 70%, 75%, 80%, 85%, 90%, 95%, or 100%. In various embodiments, the number percentage of marked hematopoietic stem cells is determined based on the fraction of marked CD34⁺ cells in bone marrow aspirate.

At least because administration of a viral gene therapy vector to a subject, including administration to a subject of any of the types of viral gene therapy vectors set forth above or elsewhere herein, can cause immunotoxicity, those of skill in the art will appreciate that immune suppression regimens disclosed herein are applicable for use in methods of in vivo gene therapy generally, regardless of the type of viral gene therapy vector administered to a subject.

Support Vector. Viral gene therapy vectors can be vectors that do not require a transposase for integration in a host cell genome and include in a single viral vector genome all sequences desired, necessary, and/or sufficient for integration and/or expression of an exogenous coding nucleic acid sequence in a target cell (“self-sufficient viral gene therapy vectors”), or can be vectors that do not include in a single viral vector genome all sequences desired, necessary, and/or sufficient for integration and/or expression of an exogenous coding nucleic acid sequence in a target cell (“supported viral gene therapy vectors”). In various instances, a supported viral gene therapy vector is administered to a subject in combination with a support vector that encodes and/or expresses an agent that facilitates integration and/or expression of an exogenous coding nucleic acid sequence of the viral vector genome of a supported viral gene therapy vector in a target cell.

In some embodiments, a supported viral gene therapy vector is a viral gene therapy vector having a viral vector genome that does not include at least one agent necessary for integration of an exogenous coding nucleic acid sequence of the viral vector genome into a target cell genome. To provide one non-limiting example, in some embodiments a viral vector genome of a supported viral gene therapy vector includes a therapeutic payload, wherein a nucleic acid including an exogenous coding nucleic acid coding sequence is flanked by transposase inverted repeats such that presence of the corresponding transposase can mediate integration of the therapeutic payload into a host cell genome. However, in certain such embodiments, the viral vector genome of the supported viral gene therapy vector does not encode the transposase, and the transpose is not otherwise or naturally present in the host cell. In certain such embodiments, a support vector administered to a subject in combination with the supported viral gene therapy vector can include a viral vector genome that encodes the corresponding transposase that can cause integration of the therapeutic payload into the host cell genome.

In certain specific embodiments a supported viral gene therapy vector genome includes a therapeutic payload this is flanked with sleeping beauty (SB) transposase inverted repeats, rendering the therapeutic payload a transposon, and the support vector encodes and expresses an SB transposase that causes integration of the therapeutic payload in a host genome. In various embodiments, the therapeutic payload transposon, inclusive of the SB transposase inverted repeats, is flanked with recombination sites that, when exposed to recombinase, cause circularization of a nucleic acid including the therapeutic payload transposon, which circularization increases the efficiency with which an SB transposase can mediate integration of the therapeutic payload into the host cell genome. In various embodiments, the SB transposase is SB10, SB11, SB100 or SB100x.

Viral vector gene therapies including a supported viral gene therapy vector and a support vector can be useful, e.g., where independent titration of agent encoded on the separate vectors is desired, or where vector capacity limitations inhibit inclusion of nucleic acid sequences encoding all desired agents in a single vector genome. Administration of viral vector gene therapies including a supported viral gene therapy vector and a support vector can require a higher dosage of support-viral gene therapy vector, e.g., as a total dose over a period of time (e.g., in a single administration, hour, day, or regimen of treatment) than viral vector gene therapies utilizing only a single vector species. As will be appreciated by those of skill in the art, a higher dose (e.g., unit dose or total dose) of a viral vector can result in induction of a more rapid, more severe, and/or more sustained immunotoxic response when administered to a subject(s) as compared to a reference including administration of a lower dose (e.g., unit dose or total dose) of viral vector (e.g., of the same vector or vectors). Accordingly, in addition to the general need for immune suppression regimens for use with viral vector gene therapies, there is a particular need for immune suppression regimens for use in viral vector gene therapies that include a supported viral gene therapy vector and a support vector.

A support vector can be a viral vector of any type, including without limitation those set forth above, e.g., an adenovirus (Ad), adeno-associated virus (AAV), herpes simplex virus (e.g., HSV, HSV1), retrovirus (e.g., MLV, MMSV, MSCV), lentivirus (e.g., HIV-1, HIV-2), alphavirus (e.g., SFV, SIN, VEE, M1), flavivirus (e.g., Kunjin, West Nile, Dengue virus), rhabdovirus (e.g., rabies, VSV), measles virus (e.g., MV-Edm), Newcastle disease virus (NDV), poxvirus, or picornavirus (e.g., coxsackieviruses). Thus, a support vector can be, for example, an AAV gene therapy vector or adenoviral gene therapy vector of any of a variety of serotypes and pseudotypes known in the art, including without limitation an Ad5, Ad35, Ad5/35, Ad35++, or Ad5/35++ vector.

In various embodiments, a viral vector gene therapy includes a supported viral gene therapy vector and a support vector, where the supported viral gene therapy vector and support vector are of the same virus type, class, serotype, or pseudotype. In various embodiments, a viral vector gene therapy includes a supported viral gene therapy vector and a support vector, where the supported viral gene therapy vector and support vector are of two different virus types, classes, serotypes, or pseudotypes. For at least the reasons set forth above and elsewhere herein, those of skill in the art will appreciate that immune suppression regimens disclosed herein are applicable for use in methods of in vivo gene therapy that include a supported viral gene therapy vector and a support vector generally, regardless of the virus type, class, serotype, or pseudotype of the supported viral gene therapy vector and the support vector, whether same or different.

In vivo Gene Therapy Regimens. In various embodiments of the present disclosure, an in vivo gene therapy includes administration of at least one viral gene therapy vector to a subject in combination with at least one immune suppression regimen. In an in vivo gene therapy including more than one vector species, such as a first vector that is a supported viral gene therapy vector in combination with a second vector that is a support vector, the first vector and the second vector can be administered in a single formulation or dosage form or in two separate formulations or dosage forms. In various embodiments, the first and second vectors can be administered at the same time or at different times, e.g., during the same one-hour period or during non-overlapping one-hour periods. In various embodiments, the first and second vectors can be administered at the same time or at different times, e.g., on the same day or on different days. In various embodiments, the first and second vectors can be administered at the same dosage or at different dosages, e.g., where the dosage is measured as the total number of viral particles or as a number of viral particles per kilogram of the subject. In various embodiments, the first and second vectors can be administered in a pre-defined ratio. In various embodiments, the ratio is in the range of 2:1 to 1:2, e.g., 1:1.

In various embodiments, a vector is administered to a subject in a single total dose on a single day. In various embodiments a vector is administered in two, three, four, or more unit doses that together constitute a total dose. In various embodiments, one unit dose of a vector is administered to a subject per day on each of one, two, three, four, or more consecutive days. In various embodiments, two unit doses of a vector are administered to a subject per day on each of one, two, three, four, or more consecutive days. Accordingly, in various embodiments, a daily dose can refer to the dose of vector received by a subject over the course of a day. In various embodiments, the term day refers to a twenty-four-hour period, such as a twenty-four-hour period from midnight of a first calendar date to midnight of the next calendar date.

In various embodiments, a unit dose, daily dose, or total dose of a vector, such as a viral gene therapy vector or support vector, or the total combined dose of a viral gene therapy vector and a support vector, can be at least 1E8, 5E8, 1E9, 5E9, 1E10, 5E10, 1E11, 5E11, 1E12, 5E12, 1E13, 5E13, 1E14, or 1E15 viral particles per kilogram (vp/kg). In various embodiments, a unit dose, daily dose, or total dose of a vector, such as a viral gene therapy vector or support vector, or the total combined dose of a viral gene therapy vector and a support vector, can fall within a range having a lower bound selected from 1E8, 5E8, 1E9, 5E9, 1E10, 5E10, 1E11, 5E11, 1E12, 5E12, 1E13, 5E13, 1E14, or 1E15 vp/kg and an upper bound selected from 1E8, 5E8, 1E9, 5E9, 1E10, 5E10, 1E11, 5E11, 1E12, 5E12, 1E13, 5E13, 1E14, or 1E15 vp/kg.

In various embodiments, a viral gene therapy vector is administered at a unit dose, daily dose, or total dose of at least 1E10, 5E10, 1E11, 5E11, 1E12, 5E12, 1E13, 5E13, 1E14, or 1E15 vp/kg and a support vector is administered at a unit dose, daily dose, or total dose of at least 1E8, 5E8, 1E9, 5E9, 1E10, 5E10, 1E11, and 5E11 vp/kg, optionally where the unit dose, daily dose, or total dose of the viral gene therapy vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the support vector is within a range having a lower bound selected from 1E8, 5E8, 1E9, 5E9, 1E10, and 5E10 vp/kg and an upper bound selected from 1E9, 5E9, 1E10, 5E10, 1E11, and 5E11 vp/kg.

In various embodiments, a support vector is administered at a unit dose, daily dose, or total dose of at least 1E10, 5E10, 1E11, 5E11, 1E12, 5E12, 1E13, 5E13, 1E14, or 1E15 vp/kg and a supported viral gene therapy vector is administered at a unit dose, daily dose, or total dose of at least 1E8, 5E8, 1E9, 5E9, 1E10, 5E10, 1E11, and 5E11 vp/kg, optionally where the unit dose, daily dose, or total dose of the support vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the supported viral gene therapy vector is within a range having a lower bound selected from 1E8, 5E8, 1E9, 5E9, 1E10, and 5E10 vp/kg and an upper bound selected from 1E9, 5E9, 1E10, 5E10, 1E11, and 5E11 vp/kg. In various embodiments, a supported viral gene therapy vector and a support vector are administered in a pre-defined ratio. In various embodiments, the ratio is in the range of 2:1 to 1:2, e.g., 1:1.

In various embodiments, an immune suppression regimen is administered to a subject that also receives at least one viral gene therapy vector, where the immune suppression regimen includes administration of at least one immune suppression agent to the subject on (i) one or more days prior to administration to the subject of a first dose of the viral gene therapy vector; (ii) on the same day as administration of a first dose of the viral gene therapy vector; (iii) on the same day as administration of one or more second or other subsequent doses of the viral gene therapy vector; and/or (iv) on any of one or more, or all, days intervening between administration to the subject of the first dose of the viral gene therapy vector and administration of any of one or more, or all, second or other subsequent doses of the viral gene therapy vector.

An immune suppression regimen administered to a subject in conjunction with a viral vector gene therapy can include an immune suppression regimen that includes any agent that is an inflammatory signal inhibitor. An immune suppression regimen administered to a subject in conjunction with a viral vector gene therapy can include immune suppression agents selected from any of 1, 2, 3, 4, 5, or 6 of (i) an inflammatory signal inhibitor, such as an interleukin-1 (IL-1) signal inhibitor; (ii) an IL-6 signal inhibitor; (iii) a corticosteroid; (iv) a calcineurin inhibitor; (v) a TNF-α signal inhibitor; and (vi) a JAK signal inhibitor; any or all of which, when present, can be administered in accordance with a distinct immune suppression agent regimen. In certain embodiments, an immune suppression regimen administered to a subject in conjunction with a viral vector gene therapy can include immune suppression agents selected from any of 1, 2, 3, or 4 of (i) an interleukin-1 (IL-1) signal inhibitor; (ii) an IL-6 signal inhibitor; (iii) a corticosteroid; and (iv) a calcineurin inhibitor; any or all of which, when present, can be administered in accordance with a distinct immune suppression agent regimen.

In an in vivo gene therapy including an immune suppression regimen that includes more than immune suppression agent, such as a first immune suppression agent and at least a second immune suppression agent of a different immune suppression agent class, each immune suppression agent can be administered in a single formulation or dosage form with one or more other immune suppression agents or in a plurality of separate formulations or dosage forms. In various embodiments, each immune suppression agent can be administered at the same time as one or more other immune suppression agents or at different times, e.g., during the same one-hour period or during non-overlapping one-hour periods. In various embodiments, each immune suppression agent can be administered at the same time or at different times as one or more other immune suppression agents, e.g., on the same day or on different days.

In various embodiments, an immune suppression agent is administered to a subject in a single total dose on a single day. In various embodiments an immune suppression agent is administered in two, three, four, or more unit doses that together constitute a total dose. In various embodiments, one unit dose of an immune suppression agent is administered to a subject per day on each of one, two, three, four, or more consecutive days. In various embodiments, two unit doses of an immune suppression agent are administered to a subject per day on each of one, two, three, four, or more consecutive days. Accordingly, in various embodiments, a daily dose can refer to the dose of immune suppression agent received by a subject over the course of a day.

In various embodiments, an immune suppression regimen includes an interleukin-1 (IL-1) signal inhibitor such as an IL-1 receptor antagonist, e.g., anakinra. In various embodiments, an interleukin-1 (IL-1) signal inhibitor such as an IL-1 receptor antagonist, e.g., anakinra, is administered to a subject (i) on the day prior to administration of a first dose of the vector; (ii) on the day of administration of a first dose of the vector; (iii) on the day of administration of one or more subsequent doses of the vector; (iv) on each day between the day of administration of a first dose of the vector and the day of administration of a last dose of the vector; and/or (v) on each of one, two, or more days after the day of administration of a last dose of the vector. In various embodiments, an interleukin-1 (IL-1) signal inhibitor such as an IL-1 receptor antagonist, e.g., anakinra, is administered to a subject (i) on the day of administration of a first dose of the vector and (ii) on the day of administration of one or more subsequent doses of the vector. In various embodiments, an IL-1 signal inhibitor, e.g., anakinra, is administered to a subject twice on each of these days, e.g., once in the morning and once in the afternoon.

In various embodiments, anakinra or another IL-1 signal inhibitor is administered 1 to 10 hours prior to one or more doses of vector (e.g., about 1, 2, 3, 4, 5, 6, 7, 8, 9, or 10 hours prior to administration of a vector, e.g., about 1 to 10, 1 to 9, 1 to 8, 1 to 7, 1 to 6, 1 to 5, 1 to 4, 1 to 3, 1 to 2, 0 to 1, 3 to 10, 3 to 9, 3 to 8, 3 to 7, 3 to 6, 3 to 5, 3 to 4, or 0 to 3 hours prior to one or more doses of vector. In certain particular embodiments, a dose of anakinra or another IL-1 signal inhibitor is administered 1 to 3 hours prior to administration of a first dose of vector. In certain particular embodiments, a dose of anakinra or another IL-1 signal inhibitor is administered 1 to 3 hours prior to administration of one or more subsequent doses of a vector.

In various embodiments anakinra or another IL-1 signal inhibitor is administered within about 1 hour prior to administration of one or more doses of vector (e.g., within about 60, 45, 30, 25, 20, 15, 10, 5, 4, 3, 2, or 1 minutes prior to administration of one or more doses of vector). In various embodiments, anakinra or another IL-1 signal inhibitor is administered intravenously. In various embodiments, anakinra or another IL-1 signal inhibitor is administered subcutaneously. In various embodiments, anakinra or another IL-1 signal inhibitor is administered subcutaneously about 1 to 10 (e.g., about 1 to 3) hours prior to administration of a first dose of vector. In various embodiments anakinra or another IL-1 signal inhibitor is administered intravenously within about 1 hour prior to administration of one or more doses of vector (e.g., within about 60, 45, 30, 25, 20, 15, 10, 5, 4, 3, 2, or 1 minutes prior to administration of one or more doses of vector).

In various embodiments, an IL-1 signal inhibitor is anakinra or another IL-1R antagonist and a daily dose of anakinra or another IL-1R antagonist is, or is at least, 0.01, 0.05, 0.1, 0.2, 0.3, 0.4, 0.5, 1, 2, 3, 4, 5, 6, 7, 8, 9, 10, 15, or 20 mg/kg/day. In certain embodiments, the dose of anakinra or another IL-1R antagonist is 0.01 to 20, 0.01 to 10, or 001 to 5 mg/kg/day. In certain embodiments, the dose of anakinra or another IL-1R antagonist is 1 to 2, 1 to 4, 1 to 6, 1 to 8, or 1 to 10 mg/kg/day. In various embodiments, an IL-1 signal inhibitor is anakinra or another IL-1R antagonist and a daily dose of anakinra or another IL-1R antagonist is 1 to 8 mg/kg/day. In certain embodiments, the dose of anakinra or another IL-1R antagonist is, or is at least, 10, 20, 30, 40, 50, 60, 70, 80, 90, 100, 125, 150, 175, or 200 mg/day. In certain embodiments, the dose of anakinra or another IL-1R antagonist is 10 to 200, 20 to 200, 30 to 175, 40 to 175, 50 to 150, 60 to 150, 80 to 125, 90 to 125, or 100 mg/day. In various embodiments, a daily dose of anakinra or another IL-1R antagonist has a range having a lower bound of 10, 20, 30, 40, 50, 60, 70, 80, 90, or 100 mg/day and an upper bound of 100, 125, 150, 175, or 20 mg/day. In various embodiments, a daily dose of anakinra or another IL-1R antagonist is 100 mg/day. In various embodiments, a daily dose is administered to a subject in two separate administrations, each at half of the daily dose, e.g., once in the morning and once in the afternoon.

Other IL-1 signal inhibitors besides anakinra include for example ADC-1001 (Alligator Bioscience), FX-201 (Flexion Therapeutics), GQ-303 (Genequine Biotherapeutics GmbH), HL-2351 (Handok, Inc.), MBIL-1RA (ProteoThera, Inc.), and human immunoglobin G or Globulin S (GC Pharma).

In various embodiments, an immune suppression regimen includes an IL-6 signal inhibitor, e.g., tocilizumab. In various embodiments, an IL-6 signal inhibitor, e.g., tocilizumab, is administered to a subject (i) on the day prior to administration of a first dose of the vector; (ii) on the day of administration of a first dose of the vector; (iii) on the day of administration of one or more subsequent doses of the vector; (iv) on each day between the day of administration of a first dose of the vector and the day of administration of a last dose of the vector; and/or (v) on each of one, two, or more days after the day of administration of a last dose of the vector. In various embodiments, an IL-6 signal inhibitor, e.g., tocilizumab, is administered to a subject (i) on the day of administration of a first dose of the vector and (ii) on the day of administration of one or more subsequent doses of the vector. In various embodiments, an IL-6 signal inhibitor, e.g., tocilizumab, is administered to a subject twice on each of these days, e.g., once in the morning and once in the afternoon. In various embodiments tocilizumab or another IL-6 signal inhibitor is administered within about 1 hour prior to administration of one or more doses of vector (e.g., within about 60, 45, 30, 25, 20, 15, 10, 5, 4, 3, 2, or 1 minutes prior to administration of one or more doses of vector).

In various embodiments, an IL-6 signal inhibitor is tocilizumab and a daily dose of tocilizumab is, or is at least, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, or 200 mg/day. In various embodiments, a daily dose of tocilizumab has a range having a lower bound of 1, 2, 3, 4, 5, 6, 7, 8, 9, 10, 11, 12, 13, 14, 15, 16, 17, 18, 19, 20 mg/day and an upper bound of 15, 16, 17, 18, 19, 20, 25, 30, 35, 40, 45, 50, 60, 70, 80, 90, 100, 110, 120, 130, 140, 150, 160, 170, 180, 190, or 200 mg/day. In various embodiments, an IL-6 signal inhibitor is tocilizumab and a daily dose of tocilizumab is, or is at least, 0.1, 0.5, 1, 2, 3, 4, 5, 6, 7, 8, 9, 10, 11, 12, 13, 14, 15, 16, 17, 18, 19, 20, 25, 30, 35, 40, 45, or 50 mg/kg/day. In various embodiments, a daily dose of tocilizumab has a range having a lower bound of 0.1, 0.5, 1, 2, 3, 4, 5, 6, 7, 8, 9, 10, 11, 12, 13, 14, 15, 16, 17, 18, 19, 20 mg/kg/day and an upper bound of 15, 16, 17, 18, 19, 20, 25, 30, 35, 40, 45, or 50 mg/kg/day. In various embodiments, a daily dose of tocilizumab has a range of 1-15, 1-12, 1-10, 1-5, 5-10, 5-12, or 5-15 mg/kg/day. In various embodiments, a dose of tocilizumab is 162 mg, e.g., a daily dose or weekly dose. In various embodiments, a daily dose is administered to a subject in two separate administrations, each at half of the daily dose, e.g., once in the morning and once in the afternoon.

Other IL-6 signal inhibitors besides tocilizumab include BCD-089 (Biocad), HS-628 (Zhejiang Hisun Pharm), and APX-007 (Apexigen).

In various embodiments, an immune suppression regimen includes a corticosteroid, e.g., dexamethasone. In various embodiments, a corticosteroid, e.g., dexamethasone, is administered to a subject (i) on the day prior to administration of a first dose of the vector; (ii) on the day of administration of a first dose of the vector; (iii) on the day of administration of one or more subsequent doses of the vector; (iv) on each day between the day of administration of a first dose of the vector and the day of administration of a last dose of the vector; and/or (v) on each of one, two, or more days after the day of administration of a last dose of the vector. In various embodiments, a corticosteroid, e.g., dexamethasone, is administered to a subject (i) on the day prior to administration of a first dose of the vector; (ii) on the day of administration of a first dose of the vector; and (iii) on the day of administration of one or more subsequent doses of the vector. In various embodiments, a corticosteroid, e.g., dexamethasone, is administered to a subject once on the first of these days, e.g., in the afternoon and twice on each of the other days, e.g., once in the morning and once in the afternoon.

In various embodiments, the corticosteroid is dexamethasone and a daily dose of dexamethasone is, or is at least, 0.01, 0.05, 0.1, 0.5, 1, 1.5, 2, 2.5, 3.0, 3.5, 4.0, 4.5, 5.0, 7.5, 10.0, 12.5, or 15 mg/kg/day. In various embodiments, a daily dose of dexamethasone has a range having a lower bound of 0.01, 0.05, 0.1, 0.5, 1, 1.5, 2, 2.5, 3.0, 3.5, 4.0, 4.5, or 5.0 mg/kg/day and an upper bound of 5.0, 7.5, 10.0, 12.5, or 15 mg/kg/day. In various embodiments, a daily dose is administered to a subject in two separate administrations, each at half of the daily dose, e.g., once in the morning and once in the afternoon.

In various embodiments, an immune suppression regimen includes a calcineurin inhibitor, e.g., tacrolimus. In various embodiments, a calcineurin inhibitor, e.g., tacrolimus, is administered to a subject (i) on the day prior to administration of a first dose of the vector; (ii) on the day of administration of a first dose of the vector; (iii) on the day of administration of one or more subsequent doses of the vector; (iv) on each day between the day of administration of a first dose of the vector and the day of administration of a last dose of the vector; and/or (v) on each of one, two, or more days after the day of administration of a last dose of the vector. In various embodiments, a calcineurin inhibitor, e.g., tacrolimus, is administered to a subject on the four days prior to administration of a first dose of the vector; (ii) on the day of administration of a first dose of the vector; (iii) on the day of administration of one or more subsequent doses of the vector; (iv) on each of two days after administration of a last dose of the vector; and, optionally, (v) on each of one, two, or more additional days. In various embodiments, a calcineurin inhibitor, e.g., tacrolimus, is administered to a subject twice on each of these days, e.g., once in the morning and once in the afternoon.

In various embodiments, the calcineurin inhibitor is tacrolimus and a daily dose of tacrolimus is, or is at least, 0.001, 0.005, 0.01, 0.02, 0.03, 0.04, 0.05, 0.1, 0.2, 0.3, 0.4, or 0.5 mg/kg/day. In various embodiments, a daily dose of tacrolimus has a range having a lower bound of 0.001, 0.005, 0.01, 0.02, 0.03, 0.04, or 0.05 mg/kg/day, and an upper bound of 0.02, 0.03, 0.04, 0.05, 0.1, 0.2, 0.3, 0.4, or 0.5 mg/kg/day. In various embodiments, a daily dose is administered to a subject in two separate administrations, each at half of the daily dose, e.g., once in the morning and once in the afternoon.

In various embodiments, an immune suppression regimen includes each of (i) an interleukin-1 (IL-1) signal inhibitor such as an IL-1 receptor antagonist; (ii) an IL-6 signal inhibitor; (iii) a corticosteroid; and (iv) a calcineurin inhibitor, as disclosed herein. In various embodiments, an immune suppression regimen includes each of (i) anakinra; (ii) tocilizumab; (iii) dexamethasone; and (iv) tacrolimus, as disclosed herein.

In various embodiments, administration of an immune suppression regimen, or an immune suppression agent thereof, is based on the measured level of an immunotoxicity biomarker, where the dosage of the immune suppression agent, or of one or more immune suppression agents of the immune suppression regimen, is increased in amount and/or frequency (e.g., increased in unit dose, daily dose, total dose, frequency of doses, and/or total number of doses), if the marker level is indicative of immunotoxicity and/or of increased immunotoxicity relative to a reference (such as an the measured level of the biomarker in an earlier sample from the same subject), decreased in amount and/or frequency if the marker level is indicative of an absence of immunotoxicity and/or of decreased immunotoxicity relative to a reference (such as an the measured level of the biomarker in an earlier sample from the same subject). Biomarkers of immunotoxicity can include any of one or more of the level of IL-Iβ, IL-2, IL-3, IL-4, IL-5, IL-6, IL-8, IL-10, IL-12, IL-13, IL-15, IL-17, IL-23, IL-27, IL-30, IL-36 IL-1Ra, IL-2R, IFN-α, IFN-b, IFN-γ, MIP-Ia, MIP-Iβ, MCP-1, TNF-α, TNF-8, GM-CSF, G-CSF, CXCL9, CXCL10, VEGF, RANTES, EGF, HGF, FGF-8, CD40, CD40L, C-reactive protein, procalcitonin, ferritin, D-dimer, total population of lymphocytes, subpopulations of lymphocytes, subject temperature, and a combination thereof. A biomarker can be measured before, during, or after administration of one or more doses of a viral gene therapy vector and/or of an immune suppression agent.

In certain embodiments, a dosing regimen of one or more immune suppression agents of the immune suppression regimen is increased in unit dose, daily dose, total dose, frequency of doses, and/or total number of doses based on the measured level of an immunotoxicity biomarker in the subject or a sample from the subject after administration of at least one dose of the viral gene therapy vector, where the dosing regimen of the one or more immune suppression agents, is increased if the measured level is indicative of significant, high, or increased immunotoxicity (e.g., as compared to a reference) and/or decreased if the measured level is indicative of low, no, or reduced immunotoxicity (e.g., as compared to a reference). In some embodiments, the immunotoxicity biomarker is selected from IL-Iβ, IL-2, IL-3, IL-4, IL-5, IL-6, IL-8, IL-10, IL-12, IL-13, IL-15, IL-17, IL-23, IL-27, IL-30, IL-36 IL-1Ra, IL-2R, IFN-α, IFN-b, IFN-γ, MIP-Ia, MIP-Iβ, MCP-1, TNF-α, TNF-β, GM-CSF, G-CSF, CXCL9, CXCL10, VEGF, RANTES, EGF, HGF, FGF-β, CD40, CD40L, C-reactive protein, procalcitonin, ferritin, D-dimer, total population of lymphocytes, subpopulations of lymphocytes, subject temperature, and a combination thereof.

In certain embodiments, a dosing regimen of one or more immune suppression agents of the immune suppression regimen is increased in unit dose, daily dose, total dose, frequency of doses, and/or total number of doses based on the measured level of antibodies to the viral gene therapy vector in the subject or a sample from the subject after administration of at least one dose of the viral gene therapy vector, where the dosing regimen of the one or more immune suppression agents, is increased if the measured level is indicative of significant, high, or increased immunotoxicity (e.g., as compared to a reference) and/or decreased if the measured level is indicative of low, no, or reduced immunotoxicity (e.g., as compared to a reference), optionally wherein the measured level is an antibody titer, and optionally wherein the antibodies are neutralizing antibodies. Means of measuring antibody levels (e.g., antibody titer) are known in the art, including without limitation enzyme-linked immunoassay (ELISA).

In various embodiments, an in vivo gene therapy regimen of the present disclosure further includes a stem cell mobilization regimen, wherein a stem cell mobilization regimen includes administering to a subject one or more agents that cause therapeutically inaccessible stem cells to become therapeutically accessible. For example, administration to a subject of a stem cell mobilization therapy can increase the circulation of hematopoietic stem cells and/or mobilize hematopoietic stem cells sequestered in bone marrow to exit bone marrow into compartments where they are accessible for in vivo transduction by viral gene therapy vectors. Hematopoietic stem cells can be target cells, e.g., of a viral gene therapy vector that binds hematopoietic stem cells, such as an adenoviral gene therapy vector that binds CD46. Exemplary stem cell mobilization agents include, without limitation, stem cell factor (SCF), small molecule VLA-4 inhibitor B105192, BOP (N-(benzenesulfonyl)-L-prolyl-L-O-(1-pyrrolidinylcarbonyl)tyrosine), heparin, granulocyte colony-stimulating factor (G-CSF), and plerixafor/AMD3100.

In various embodiments, the stem cell mobilization regimen includes administration of G-CSF and plerixafor/AMD3100. In various embodiments G-CSF is administered to a subject (i) daily on the four days prior to administration of a first dose of the vector; (ii) on the day of administration of a first dose of the vector; and (iii) on the day of administration of one or more subsequent doses of the vector. In various embodiments plerixafor/AMD3100 is administered to a subject (i) on the day prior to administration of a first dose of the vector and (ii) on the day of administration of a first dose of the vector. In various embodiments G-CSF is administered once daily at a dose that is, or is at least, 10, 20, 30, 40, 50, 75, 100, 150, or 200 ug/kg. In various embodiments, a daily dose of G-CSF has a range having a lower bound of 10, 20, 30, 40, 50, or 75 ug/kg/day and an upper bound of 100, 150, or 200 ug/kg/day. In various embodiments plerixafor/AMD3100 is administered once daily at a dose that is, or is at least, 1, 2, 3, 4, 5, 7.5, 10, 15, or 20 mg/kg. In various embodiments, a daily dose of G-CSF has a range having a lower bound of 1, 2, 3, 4, 5, or 7.5 mg/kg/day and an upper bound of 10, 15, or 20 mg/kg/day.

Various Embodiments. In various embodiments of the present disclosure, an in vivo gene therapy includes administration of at least one viral gene therapy vector, e.g., an adenoviral gene therapy vector of the present disclosure (such as a supported adenoviral gene therapy vector in combination with an adenoviral support vector as described herein including helper dependent versions of these, e.g., that bind CD46 such as Ad5, Ad35, Ad5/35, Ad35++ and Ad5/35++, e.g., where the two vectors are administered together in a 1:1 ratio on two consecutive days, such as in the morning of each of these days) to a subject in combination with:

(a) an immune suppression regimen that includes (i) an inflammatory signal inhibitor, such as an interleukin-1 (IL-1) signal inhibitor, e.g., anakinra (such as on the day of administration of a first dose of the vector and on the day of administration of one or more subsequent doses of the vector, e.g., twice on each of these days, e.g., once in the morning and once in the afternoon, wherein the administered daily dose(s) are as described herein); (ii) an IL-6 signal inhibitor, e.g., tocilizumab (such as on the day of administration of a first dose of the vector and on the day of administration of one or more subsequent doses of the vector, e.g., twice on each of these days, e.g., once in the morning and once in the afternoon, wherein the administered daily dose(s) are as described herein); (iii) a corticosteroid, e.g., dexamethasone (such as on the day prior to administration of a first dose of the vector; on the day of administration of a first dose of the vector; and on the day of administration of one or more subsequent doses of the vector, e.g., once on the first of these days, e.g., in the afternoon and twice on each of the other days, e.g., once in the morning and once in the afternoon, wherein the administered daily dose(s) are as described herein); and (iv) a calcineurin inhibitor, e.g., tacrolimus (such as on the four days prior to administration of a first dose of the vector; on the day of administration of a first dose of the vector; on the day of administration of one or more subsequent doses of the vector; on each of two days after administration of a last dose of the vector; and, optionally, on each of one, two, or more additional day, e.g., where it is administered to a subject twice on each of these days, e.g., once in the morning and once in the afternoon, wherein the administered daily dose(s) are as described herein);
(b) a stem cell mobilization regimen such as a regimen that increases the circulation of hematopoietic stem cells and/or mobilizes hematopoietic stem cells sequestered in bone marrow to exit bone marrow into compartments where they are accessible for in vivo transduction by the vector, e.g., a stem cell mobilization regimen that includes G-CSF and plerixafor/AMD3100 such as a regimen where (i) G-CSF is administered to the subject daily on the four days prior to administration of a first dose of the vector; on the day of administration of a first dose of the vector; and on the day of administration of one or more subsequent doses of the vector, e.g., where G-CSF is administered once on each of these days such as in the morning, wherein the administered daily dose(s) are as described herein; and (ii) plerixafor/AMD3100 is administered to the subject on the day prior to administration of a first dose of the vector and on the day of administration of a first dose of the vector, e.g., where plerixafor/AMD3100 is administered once on each of these days such as in the afternoon (or 9 to 11 hours prior to the first and second doses of the vector), wherein the administered daily dose(s) are as described herein; and
(c) a selection regimen such as a regimen that selects for hematopoietic stem cells that have been in vivo transduced by the vector, e.g., a selection regimen that includes O⁶-benzylguanine (O⁶BG) and 1,3-bis(2-chloroethyl)-1-nitroso-urea (BCNU) (O⁶BG/BCNU), such as a regimen where O⁶BG/BCNU is administered at week 4, week 6 (optionally), and week 8 (optionally) after the day of administration of a first dose of the vector (and optionally at additional 2 week intervals thereafter if needed to further select for transduced cells).

Formulation and Administration. A vector can be formulated such that it is pharmaceutically acceptable for administration to cells or animals, e.g., to humans. A vector may be administered in vivo. In various instances, a vector can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In various embodiments, a composition including a vector as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80TM, HCO-50 and the like.

As disclosed herein, a composition can be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, a vector can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion. A parenteral route of administration can be, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a vector of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A vector can be administered parenterally in the form of an injectable formulation including a sterile solution or suspension in water or another pharmaceutically acceptable liquid. For example, the vector can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of vector included in the pharmaceutical preparations is such that a suitable dose within the designated range is provided. Non-limiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

In some embodiments, a vector described herein can be therapeutically delivered to a subject by way of local administration. As used herein, “local administration” or “local delivery,” can refer to delivery that does not rely upon transport of the vector or vector to its intended target tissue or site via the vascular system. For example, the vector may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

Pharmaceutical forms of vector formulations suitable for injection can include sterile aqueous solutions or dispersions. A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Preferably, isotonic agents, for example, sugars or sodium chloride can be used in the formulations.

In addition, one skilled in the art may also contemplate additional delivery method may be via electroporation, sonophoresis, intraosseous injections methods or by using gene gun. Vectors may also be implanted into microchips, nano-chips or nanoparticles.

A suitable dose of a vector described herein can depend on a variety of factors including, e.g., the age, sex, and weight of a subject to be treated, the condition or disease to be treated, and the particular vector used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. A suitable means of administration of a vector can be selected based on the condition or disease to be treated and upon the age and condition of a subject. Dose and method of administration can vary depending on the weight, age, condition, and the like of a patient, and can be suitably selected as needed by those skilled in the art. A specific dosage and treatment regimen for any particular subject can be adjusted based on the judgment of a medical practitioner.

A vector solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount can be an amount at which any toxic or detrimental effects of the composition are outweighed by therapeutically beneficial effects.

Immune suppression agents of the present disclosure can be formulated individually or together in any of the various forms provided herein or otherwise known in the art. In various embodiments, immune suppression agents described herein can be formulated in a pharmaceutical composition. Pharmaceutical compositions can be formulated by methods known to those skilled in the art (such as described in Remington's Pharmaceutical Sciences, 17th edition, ed. Alfonso R. Gennaro, Mack Publishing Company, Easton, Pa. (1985)).

In various instances, an immune suppression agent pharmaceutical composition can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In various embodiments, a pharmaceutical composition including an immune suppression agent as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80TM, HCO-50 and the like.

As disclosed herein, an immune suppression agent pharmaceutical composition may be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories. Selection or use of any particular form may depend, in part, on the intended mode of administration and therapeutic application. For example, compositions containing a composition intended for systemic or local delivery can be in the form of injectable or infusible solutions. Accordingly, the compositions can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion. Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

In various embodiments, an immune suppression agent pharmaceutical composition of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A pharmaceutical composition can be administered parenterally in the form of an injectable formulation including a sterile solution or suspension in water or another pharmaceutically acceptable liquid. For example, the pharmaceutical composition can be formulated by suitably combining the immune suppression agent with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of immune suppression agent included in the pharmaceutical preparations is such that a suitable dose within the designated range is provided. Non-limiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

Compositions including one or more immune suppression agents as described herein can be formulated in immunoliposome compositions. Such formulations can be prepared by methods known in the art. Liposomes with enhanced circulation time are disclosed in, e.g., U.S. Pat. No. 5,013,556.

In certain embodiments, compositions can be formulated with a carrier that will protect the immune suppression agent against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for combining with an immune suppression agent for subcutaneous injection.

In some embodiments, a composition described herein can be therapeutically delivered to a subject by way of local administration. As used herein, “local administration” or “local delivery,” can refer to delivery that does not rely upon transport of the composition or agent to its intended target tissue or site via the vascular system. For example, the composition may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

A pharmaceutical solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount of a composition described herein can also vary according to factors such as the disease state, age, sex, and weight of the individual, and the ability of the composition (and one or more additional active agents) to elicit a desired response in the individual, e.g., amelioration of at least one condition parameter, e.g., amelioration of at least one symptom of the complement-mediated disorder. For example, a therapeutically effective amount of a composition described herein can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

A suitable dose of an immune suppression agent composition described herein, which dose is capable of treating or preventing a disorder in a subject, can depend on a variety of factors including, e.g., the age, sex, and weight of a subject to be treated and the particular inhibitor compound used. Other factors affecting the dose administered to the subject include, e.g., the type or severity of the disorder. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of the treating medical practitioner.

In various embodiments, an immune suppression regimen includes any or all of (i) an interleukin-1 (IL-1) signal inhibitor such as an IL-1 receptor antagonist; (ii) an IL-6 signal inhibitor; (iii) a corticosteroid; and (iv) a calcineurin inhibitor, as disclosed herein, where each can be independently formulated for administration, and/or administered, by injection, e.g., intravenously or subcutaneously. In various embodiments, an immune suppression regimen includes any or all of (i) anakinra; (ii) tocilizumab; (iii) dexamethasone; and (iv) tacrolimus, as disclosed herein, where each can be independently formulated for administration, and/or administered, by injection, e.g., intravenously or subcutaneously. For the avoidance of doubt, for any combination of a plurality of immune suppression agents provided herein in an immune suppression regimen, each of the immune suppression agents can be each can be independently formulated for administration, and/or administered, by injection, e.g., intravenously or subcutaneously.

Applications of Immune Suppression Regimens. As will be appreciated by those of skill in the art, gene therapy is a platform with many uses, and platforms of gene therapy must also be understood to have both general applicability in the field of gene therapy as well as specific applicability to many individual applications. Due to the disadvantages of ex vivo methods of engineering stem cells for use in methods of gene therapy, including without limitation prohibitive cost and technical complexity, improved methods of in vivo gene therapy as set forth herein have broad and potentially transformative value in the field of gene therapy. Notwithstanding the readily apparent general applicability of the presently disclosed methods to the field of gene therapy, several exemplary specific applications are set forth herein.

In certain exemplary applications, an immune suppression regimen can be used in combination with a viral gene therapy vector that transduces hematopoietic stem cells, optionally in further combination with a stem cell mobilization regimen that mobilizes hematopoietic stem cells from bone marrow. Hematopoietic stem cells can be transduced, e.g., by an adenoviral gene therapy vector, e.g., an adenoviral gene therapy vector that targets CD46. In various embodiments, transduction of hematopoietic stem cells can be used as a means to treat various particular diseases, e.g., sickle cell anemia, thalassemia, thalassemia intermedia, hemophilia A, hemophilia B, von Willebrand Disease, Factor V Deficiency, Factor VII Deficiency, Factor X Deficiency, Factor XI Deficiency, Factor XII Deficiency, Factor XIII Deficiency, Bernard-Soulier Syndrome, or Gray Platelet Syndrome. For example, an adenoviral vector that targets CD46 can be used to treat thalassemia or thalassemia intermedia by delivering a therapeutic payload that expresses, and/or increases expression of, β-globin and/or γ-globin to hematopoietic stem cells. In another example, an adenoviral vector that targets CD46 can be used to treat hemophilia (e.g., hemophilia A or hemophilia B), by delivering a therapeutic payload that expresses, and/or increases expression of, Factor VIII or Factor IX in hematopoietic stem cells. In another example, an adenoviral vector that targets CD46 can be used to treat sickle cell anemia by delivering a therapeutic payload for correction of a genetic lesion that causes sickle cell anemia by gene editing. Exemplary applications of viral gene therapy vectors are further disclosed in, e.g., U.S. Provisional Patent Application No. 62/869,907, filed Jul. 2, 2019, which is incorporated herein by reference in its entirety, and particularly with respect to viral gene therapy vectors and applications of viral gene therapy.

The present disclosure encompasses the understanding that viral gene therapy that includes a viral gene therapy vector and an immune suppression regimen of the present disclosure reduces immunotoxicity and/or inflammation caused in subjects receiving viral gene therapy, e.g., as compared to a reference that does not include the immune suppression regimen or an agent thereof. Included therein is the understanding that the reduced immunotoxicity and/or inflammation caused by viral gene therapy that includes a viral gene therapy vector and an immune suppression regimen of the present disclosure includes a reduction in the level of one or more biomarkers of immunotoxicity and/or inflammation, e.g., as compared to a reference that does not include the immune suppression regimen or an agent thereof. Biomarkers of inflammation include, without limitation, IFN-g, TNF, IL-2, IL-4, IL-5, and IL-6. Thus, the present disclosure encompasses that any of one or more of IFN-g, TNF, IL-2, IL-4, IL-5, and IL-6 can be decreased (e.g., significantly decreased, e.g., by a p value of less than 0.05) in a subject receiving a viral gene therapy that includes a viral gene therapy vector and an immune suppression regimen of the present disclosure as compared to a reference such as a subject receiving a viral gene therapy that includes the viral gene therapy vector but does not include the immune suppression regimen or an agent thereof. In various embodiments, a qualitative or quantitative change in the level, rate of change in the level of, or variability of the level of any of one or more of IFN-g, TNF, IL-2, IL-4, IL-5, and IL-6 is determined by a method known in the art, including, e.g., ELISA or cytokine bead array.

Kits. In various embodiments the present disclosure also provides kits for performing an in vivo gene therapy method in accordance with the methods of the present disclosure. For example, a kit may include containers (optionally with written instructions, e.g., for use in in vivo gene therapy) that include immune suppression agents selected from any of 1, 2, 3, 4, 5, or 6 of (i) an inflammatory signal inhibitor, such as an interleukin-1 (IL-1) signal inhibitor e.g., anakinra; (ii) an IL-6 signal inhibitor, e.g., tocilizumab; (iii) a corticosteroid, e.g., dexamethasone; (iv) a calcineurin inhibitor, e.g., tacrolimus; (v) a TNF-α signal inhibitor; and (vi) a JAK signal inhibitor; any or all of which, when present, can be provided in unit dosage forms that correspond to the daily or other dose(s) as described herein or half-daily doses. In certain examples, a kit may include containers (optionally with written instructions, e.g., for use in in vivo gene therapy) that include immune suppression agents selected from any of 1, 2, 3, or 4 of (i) an interleukin-1 (IL-1) signal inhibitor e.g., anakinra; (ii) an IL-6 signal inhibitor, e.g., tocilizumab; (iii) a corticosteroid, e.g., dexamethasone; and (iv) a calcineurin inhibitor, e.g., tacrolimus; any or all of which, when present, can be provided in unit dosage forms that correspond to the daily or other dose(s) as described herein or half-daily doses. In various embodiments, the kits may also include containers that include stem cell mobilization agents that increase the circulation of hematopoietic stem cells and/or mobilize hematopoietic stem cells sequestered in bone marrow to exit bone marrow into compartments where they are accessible for in vivo transduction by a vector, e.g., G-CSF and plerixafor/AMD3100; any or all of which, when present, can be provided in unit dosage forms that correspond to the daily or other dose(s) as described herein. In various embodiments, the kits may also include containers that include selecting agents such as those that select for hematopoietic stem cells that have been in vivo transduced by a vector, e.g., O6-benzylguanine (O⁶BG) and 1,3-bis(2-chloroethyl)-1-nitroso-urea (BCNU) (O⁶BG/BCNU); any or all of which, when present, can be provided in unit dosage forms that correspond to the daily or other dose(s) as described herein.

### Exemplary Embodiments

1. A method of in vivo gene therapy in a mammalian subject, the method including: administering to the subject an immune suppression regimen including an inflammatory signal inhibitor; and administering to the subject at least one dose of a viral gene therapy vector.

2. A method of transducing stem cells of a mammalian subject without removal of the stem cells from the subject, the method including delivering a viral gene therapy vector to a subject having been administered an immune suppression regimen including an inflammatory signal inhibitor.

3. The method of embodiment 1 or 2, wherein the inflammatory signal inhibitor includes an interleukin-1 (IL-1) signal inhibitor, optionally wherein the IL-1 signal inhibitor includes an IL-1 receptor (IL-1R) antagonist.

4. The method of embodiment 3, wherein the IL-1R antagonist includes anakinra.

5. The method of any one of embodiments 1-4, wherein the immune suppression regimen further includes an interleukin 6 (IL-6) receptor antagonist.

6. The method of embodiment 5, wherein the IL-6 receptor antagonist includes tocilizumab.

7. The method of any one of embodiments 1-6, wherein the immune suppression regimen further includes a corticosteroid.

8. The method of embodiment 7, wherein the corticosteroid includes dexamethasone.

9. The method of any one of embodiments 1-8, wherein the immune suppression regimen further includes a calcineurin inhibitor.

10. The method of embodiment 9, wherein the calcineurin inhibitor includes tacrolimus.

11. The method of any one of embodiments 1-10, wherein the immune suppression regimen further includes a TNF-α signal inhibitor.

12. The method of embodiment 11, wherein the TNF-α signal inhibitor includes etanercept, infliximab, adalimumab, certolizumab, pegol, and/or golimumab.

13. The method of any one of embodiments 1-12, wherein the immune suppression regimen further includes a JAK signal inhibitor.

14. The method of embodiment 13, wherein the JAK signal inhibitor includes baricitinib, tofacitinib, ruxolitinib, and/or filgotinib.

15. The method of any one of embodiments 1-14, wherein the administering of the immune suppression regimen includes administering the IL-1 receptor antagonist to the subject: on the day prior to administration of a first dose of the vector; on the day of administration of a first dose of the vector; on the day of administration of one or more subsequent doses of the vector; on each day between the day of administration of a first dose of the vector and the day of administration of a last dose of the vector; and/or on each of one, two, or more days after the day of administration of a last dose of the vector; optionally wherein the IL-1 receptor antagonist includes anakinra.

16. The method of any one of embodiments 1-15, wherein the administering of the immune suppression regimen includes administering to the subject a single dose of IL-1 receptor antagonist per day or a plurality of doses of IL-1 receptor antagonist per day, optionally wherein the IL-1 receptor antagonist includes anakinra.

17. The method of embodiment 15 or 16, wherein the administering of the immune suppression regimen includes administering to the subject 0.01 to 20 mg/kg/day anakinra, optionally wherein the administration includes intravenous administration.

18. The method of embodiment 15 or 16, wherein the administering of the immune suppression regimen includes administering to the subject 10 to 200 mg/day anakinra, optionally wherein the administration includes intravenous administration.

19. The method of any one of embodiments 1-18, wherein the administering of the immune suppression regimen includes administering an IL-6 receptor antagonist to the subject: on the day prior to administration of a first dose of the vector; on the day of administration of a first dose of the vector; on the day of administration of one or more subsequent doses of the vector; on each day between the day of administration of a first dose of the vector and the day of administration of a last dose of the vector; and/or on each of one, two, or more days after the day of administration of a last dose of the vector; optionally wherein the IL-6 receptor antagonist includes tocilizumab.

20. The method of any one of embodiments 1-19, wherein the administering of the immune suppression regimen includes administering to the subject a single dose of IL-6 receptor antagonist per day or a plurality of doses of IL-6 receptor antagonist per day, optionally wherein the IL-6 receptor antagonist includes tocilizumab.

21. The method of embodiment 19 or 20, wherein the administering of the immune suppression regimen includes administering to the subject 1-15 mg/kg/day tocilizumab, 1-12 mg/kg/day tocilizumab, 1-10 mg/kg/day tocilizumab, or 5-200 mg/day tocilizumab, optionally wherein the administration includes intravenous administration.

22. The method of any one of embodiments 1-21, wherein the administering of the immune suppression regimen includes administering a corticosteroid to the subject: on the day prior to administration of a first dose of the vector; on the day of administration of a first dose of the vector; on the day of administration of one or more subsequent doses of the vector; on each day between the day of administration of a first dose of the vector and the day of administration of a last dose of the vector; and/or on each of one, two, or more days after the day of administration of a last dose of the vector; optionally wherein the corticosteroid includes dexamethasone.

23. The method of any one of embodiments 1-22, wherein the administering of the immune suppression regimen includes administering to the subject a single dose of corticosteroid per day or a plurality of doses of corticosteroid per day, optionally wherein the corticosteroid includes dexamethasone.

24. The method of embodiment 22 or 23, wherein the administering of the immune suppression regimen includes administering to the subject 0.1-10 mg/kg/day dexamethasone, optionally wherein the administration includes intravenous administration.

25. The method of any one of embodiments 1-24, wherein the administering of the immune suppression regimen includes administering a calcineurin inhibitor to the subject: on each of the four days prior to administration of a first dose of the vector; on the day of administration of a first dose of the vector; on the day of administration of one or more subsequent doses of the vector; and/or on each day between the day of administration of a first dose of the vector and the day of administration of a last dose of the vector; and/or on each of one, two, or more days after the day of administration of a last dose of the vector; optionally wherein the calcineurin inhibitor includes tacrolimus.

26. The method of any one of embodiments 1-25, wherein the administering of the immune suppression regimen includes administering to the subject a single dose of calcineurin inhibitor per day or a plurality of doses of calcineurin inhibitor per day, optionally wherein the calcineurin inhibitor includes tacrolimus.

27. The method of embodiment 25 or 26, wherein the administering of the immune suppression regimen includes administering to the subject 0.001-0.1 mg/kg/day tacrolimus, optionally wherein the administration includes subcutaneous administration.

28. The method of any one of embodiments 1-27, wherein the method (i) does not cause a significant increase in the amount of one or more of IFN-g, TNF, IL-2, IL-4, IL-5, or IL-6; or (ii) causes a significantly smaller increase in the amount of one or more of IFN-g, TNF, IL-2, IL-4, IL-5, or IL-6 as compared to a control that does not include one or more immune suppression agents, optionally wherein the control does not include one or more immune suppression agents selected from (a) the inflammatory signal inhibitor; (b) the IL-6 receptor antagonist; (c) the corticosteroid; and (d) the calcineurin inhibitor; optionally wherein the amount is measured by ELISA and/or a cytokine bead array.

29. The method of any one of embodiments 1-28, wherein the method further includes administering to the subject a stem cell mobilization regimen.

30. The method of any one of embodiments 1-29, wherein the vector includes a nucleic acid sequence that encodes a selectable marker, optionally wherein the selectable marker includes MGMT^{P140K}.

31. The method of embodiment 30, wherein the method includes administering to the subject a selecting agent, optionally wherein the selectable marker includes MGMT^{P140K} and the selecting agent includes O⁶BG/BCNU.

32. The method of embodiment 30 or 31, wherein the selecting agent is administered to the subject in one or more doses, optionally wherein a first dose of the selecting agent is administered to the subject about 1 week, 2 weeks, 3 weeks, 4 weeks, 5 weeks, 6 weeks, 7 weeks, 8 weeks, 9 weeks, and/or 10 weeks after administration of a first dose of the vector to the subject.

33. The method of any one of embodiments 1-32, wherein the vector is administered to the subject by injection, optionally wherein the injection includes intravenous or subcutaneous administration.

34. The method of any one of embodiments 1-33, wherein at least a first dose of the vector includes at least 1E10, 1E11, or 1E12 viral particles per kilogram (vp/kg).

35. The method of any one of embodiments 1-34, wherein the vector is administered at a total dosage of at least 1E10, 1E11, 1E12, 2E12, or 3E12 vp/kg.

36. The method of any one of embodiments 1-35, wherein the vector includes an adenoviral vector, adeno-associated viral vector, herpes simplex viral vector, retroviral vector, lentiviral vector, alphaviral vector, flaviviral vector, rhabdoviral vector, measles viral vector, Newcastle disease viral vector, poxviral vector, or picornaviral vector.

37. The method of any one of embodiments 1-35, wherein the vector includes an adenoviral vector.

38. The method of any one of embodiments 1-37, wherein the vector includes a group B adenoviral vector.

39. The method of any one of embodiments 1-38, wherein the vector includes, or is derived from, an Ad5/35 or Ad35 adenoviral vector, optionally wherein the vector includes an Ad35⁺⁺ or Ad5/35⁺⁺ adenoviral vector.

40. The method of any one of embodiments 1-39, wherein the vector includes a replication incompetent vector, optionally wherein the replication incompetent vector includes a helper-dependent adenoviral vector.

41. The method of any one of embodiments 1-40, wherein viral gene therapy vector includes a nucleic acid including a therapeutic payload, and wherein the method further includes administering to the subject a support vector encoding an agent that facilitates integration of the therapeutic payload into a target cell genome.

42. The method of embodiment 41, wherein the support vector is administered to the subject together with the viral gene therapy vector.

43. The method of embodiment 41 or 42, wherein the support vector is administered at a total dosage of 1E9 to 1E14 viral particles per kilogram (vp/kg).

44. The method of any one of embodiments 1-43, wherein the viral gene therapy vector includes a nucleic acid including a therapeutic payload, and wherein the method causes delivery of the therapeutic payload to stem cells, optionally wherein delivery of the therapeutic payload includes integration of the therapeutic payload into the genomes of the stem cells.

45. The method of any one of embodiments 1-44, wherein the viral gene therapy vector includes a nucleic acid including a protein-encoding therapeutic payload, and, after administration of the vector to the subject, at least about 70%, about 80%, or about 90% of PBMCs of the subject express the protein.

46. The method of any one of embodiments 1-45, wherein the subject is a human subject.

47. The method of embodiment 46, wherein the human subject suffers from sickle cell anemia, thalassemia, thalassemia intermedia, hemophilia A, hemophilia B, von Willebrand Disease, Factor V Deficiency, Factor VII Deficiency, Factor X Deficiency, Factor XI Deficiency, Factor XII Deficiency, Factor XIII Deficiency, Bernard-Soulier Syndrome, Gray Platelet Syndrome.

48. The method of any one of embodiments 1-47, wherein the dosing regimen of one or more immune suppression agents of the immune suppression regimen is increased in unit dose, daily dose, total dose, frequency of doses, and/or total number of doses based on the measured level of an immunotoxicity biomarker in the subject or a sample from the subject after administration of at least one dose of the viral gene therapy vector, where the dosing regimen of the one or more immune suppression agents, is increased if the measured level is indicative of immunotoxicity.

49. The method of embodiment 48, wherein the immunotoxicity biomarker includes IL-Iβ, IL-2, IL-3, IL-4, IL-5, IL-6, IL-8, IL-10, IL-12, IL-13, IL-15, IL-17, IL-23, IL-27, IL-30, IL-36 IL-1Ra, IL-2R, IFN-α, IFN-b, IFN-γ, MIP-Ia, MIP-Iβ, MCP-1, TNF-α, TNF-β GM-CSF, G-CSF, CXCL9, CXCL10, VEGF, RANTES, EGF, HGF, FGF-β, CD40, CD40L, C-reactive protein, procalcitonin, ferritin, D-dimer, total population of lymphocytes, subpopulations of lymphocytes, subject temperature, and/or a combination thereof.

50. The method of any one of embodiments 1-49, wherein the dosing regimen of one or more immune suppression agents of the immune suppression regimen is increased in unit dose, daily dose, total dose, frequency of doses, and/or total number of doses based on the measured level of antibodies to the viral gene therapy vector in the subject or a sample from the subject after administration of at least one dose of the viral gene therapy vector, where the dosing regimen of the one or more immune suppression agents, is increased if the measured level is indicative of immunotoxicity, optionally wherein the measured level is an antibody titer, and optionally wherein the antibodies are neutralizing antibodies.

51. The method of any one of embodiments 48-50, wherein the dosing regimen of the one or more immune suppression agents of the immune suppression regimen includes a dosing regimen of one or more of (i) an interleukin-1 (IL-1) signal inhibitor, optionally wherein the IL-1 signal inhibitor includes anakinra; (ii) an IL-6 signal inhibitor, optionally wherein the IL-6 signal inhibitor is tocilizumab; (iii) a corticosteroid, optionally wherein the corticosteroid includes dexamethasone; and (iv) a calcineurin inhibitor, optionally wherein the calcineurin inhibitor includes tacrolimus.

### EXAMPLES

### Example 1: Exemplary Scheme for In Vivo Gene Therapy Including a Viral Vector, a Support Vector, and an Immune Suppression Regimen

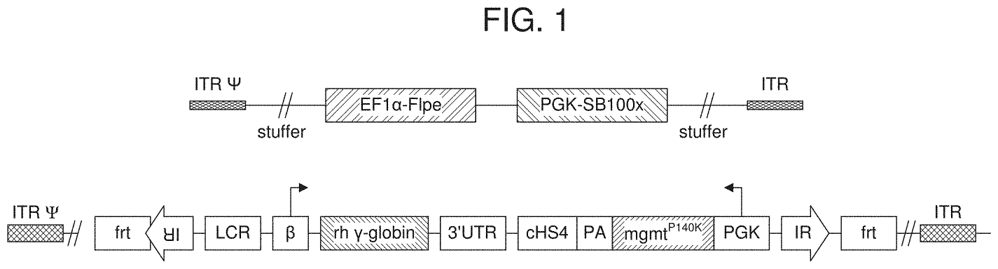

The present example provides a protocol for in vivo gene therapy that includes a viral gene therapy vector and a support vector. As shown in FIG. 1, the HDAd support vector encodes (i) a Flpe recombinase operably linked to an EF1α promoter and (ii) a transposase (SB100x) operably linked to a PGK promoter, positioned between adenoviral inverted terminal repeats (ITRs). A stuffer is also included in the support vector to produce a viral vector genome of a size efficiently packaged by adenovirus. The HDAd viral gene therapy vector includes a therapeutic payload that includes a nucleic acid sequence encoding a therapeutic protein (rh γ-globin) that is operably linked to a both a β-globin promoter and a β-globin locus control region (LCR), and that is further operably linked to 3′ UTR and chicken hypersensitive site 4 (cHS4; the chicken β-like globin gene cluster) regulatory regions. The viral gene therapy vector further (optionally) includes a nucleic acid sequence encoding an MGMT^{P140K} selectable marker operably linked to a PGK promoter. The therapeutic payload is flanked by inverted repeats (IRs) that are SB100x targets for transposition, whereby the therapeutic payload can be integrated into a host cell genome. The IRs are in turn flanked by frt sites that, upon exposure to Flpe, circularize the flanked nucleic acid to facilitate transposition of the therapeutic payload. The viral gene therapy vector further includes adenoviral ITRs.

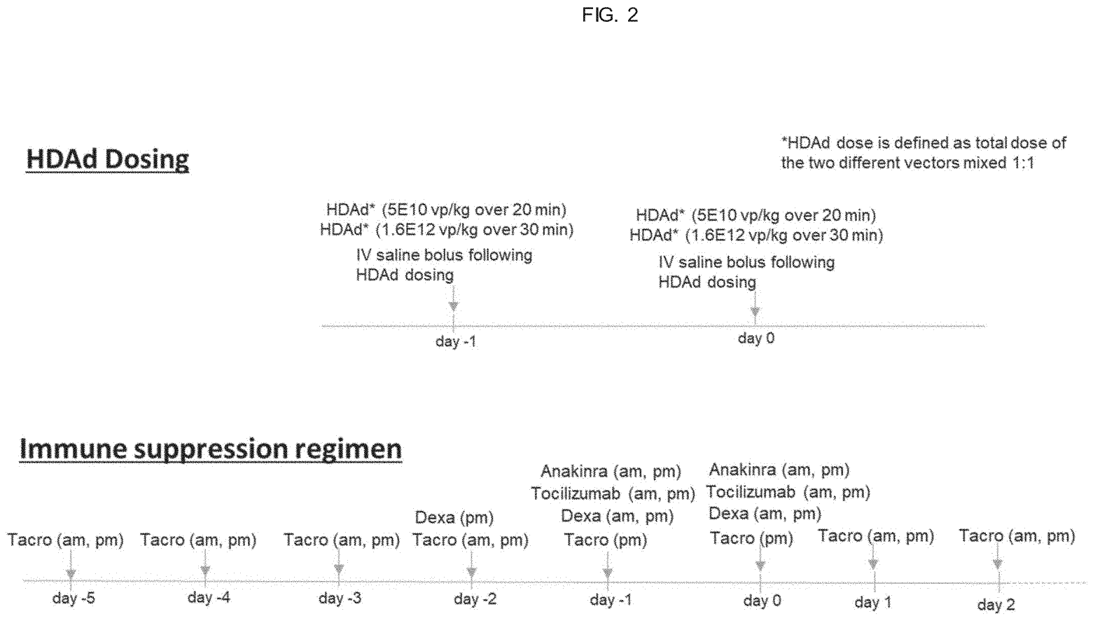

As shown in FIG. 2, the adenoviral viral gene therapy vector and support vector are mixed (in a 1:1 ratio) into a single formulation for administration to a subject. The subject receives two doses of the HDAd combination formulation on each of two consecutive days. The first dose is a dose of 5E10 vp/kg administered to the subject over a 20-minute period. The second dose administered later on the same day is a dose of 1.6E12 vp/kg administered to the subject over a 30-minute period. Accordingly, the total daily dose of the two vectors combined (in a 1:1 ratio) is 1.65E12 vp/kg. All doses are administered intravenously and followed by intravenous administration of a saline bolus. The viral gene therapy vector regimen set forth in FIG. 2 is further described in Table 1 below.

An immune suppression regimen is administered based on the timing of HDAd administration. The immune suppression regimen set forth in FIG. 2 includes anakinra, tocilizumab, tacrolimus, and dexamethasone. The immune suppression regimen set forth in FIG. 2 is further described in Table 1.

TABLE 1
Exemplary immune suppression regimen

| | Dosing | Route of | Timing (day(s)) |
|---|---|---|---|
| Agent | Scheme | Administration | of Administration |
| Dexamethasone | 2 mg/kg b.i.d. | i.v. | −1, 0 |
| Tacrolimus | 0.01 mg/kg | s.c. | −5, −4, −3, −2, −1, 0, 1, 2, |
| | b.i.d. | | and optionally for |
| | | | additional days after day 2 |
| Tocilizumab | 8 mg/kg b.i.d. | i.v. | −1, 0 |
| Anakinra | 50 mg/animal | i.v. | −1, 0 |
| | b.i.d. | | |
| Saline | 8 ml/kg | i.v. bolus (over 15 min | −1 and 0 |
| | | following each HDAd dosing) | |
| HDAd | 1.65E12 vp/kg | i.v. | −1 |
| HDAd | 1.65E12 vp/kg | i.v. | 0 |

### Example 2: Exemplary Scheme for In Vivo Gene Therapy Including a Viral Vector, a Support Vector, an Immune Suppression Regimen, and a Selecting Agent

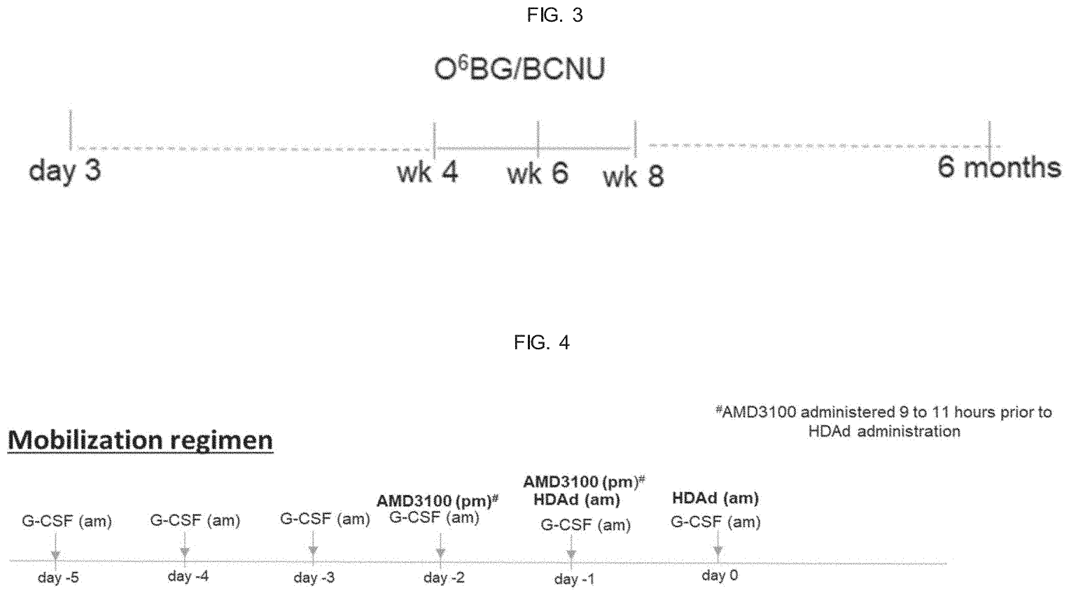

The present Example adds to the protocol set forth in Example 1. The present Example provides that the HDAd viral gene therapy vector affirmatively includes the MGMT^{P140K} selectable marker as shown in FIG. 1 and indicated as optional in Example 1. Accordingly, the present Example further provides a selection regimen that includes a selecting agent, O⁶BG/BCNU, as shown in FIG. 3. The selection regimen shown in FIG. 3 is further described in Table 2.

TABLE 2
Exemplary selection regimen

| | Dosing | Route of | Timing (week(s)) |
|---|---|---|---|
| Agent | Scheme | Administration | of Administration |
| O⁶BG/BCNU | 10, 20, and 30 mg/m² in dose #1, | i.v. | 4, 6, 8 |
| | #2, and #3, respectively | | |

### Example 3: Exemplary Scheme for In Vivo Gene Therapy Including a Viral Vector, a Support Vector, an Immune Suppression Regimen, a Selecting Agent, and a Stem Cell Mobilization Regimen

The present Example adds to the protocol set forth in Example 1 and/or the protocol set forth in Example 2. The present Example provides a stem cell mobilization regimen that can be administered to a subject to improve transduction of stem cells that typically reside in bone marrow, including hematopoietic stem cells. As shown in FIG. 4, an exemplary stem cell mobilization regimen includes G-CSF and AMD3100. The selection regimen shown in FIG. 4 is further described in Table 3.

TABLE 3
Exemplary stem cell mobilization regimen

| | Dosing | Route of | Timing (day(s)) |
|---|---|---|---|
| Agent | Scheme | Administration | of Administration |
| G-CSF | 50 ug/kg q.d. | s.c. | −5, −4, −3, −2, −1, 0 |
| AMD3100 | 5 mg/kg q.d. | s.c. | −2, −1 |
| | | | (administered 9 to 11 hours |
| | | | prior to HDAd dosing) |

### Example 4: Exemplary Scheme for In Vivo Gene Therapy Including a Viral Vector, a Support Vector, an Immune Suppression Regimen, a Selecting Agent, and a Stem Cell Mobilization Regimen

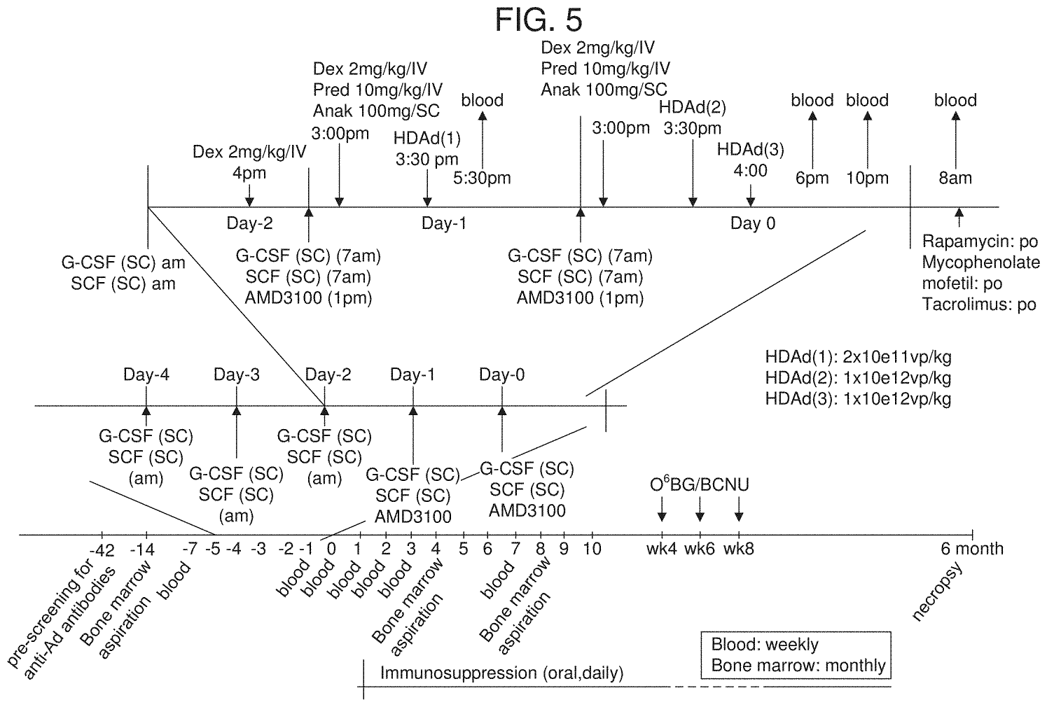

The present Example describes an alternative scheme which is shown in FIG. 5 and described below.

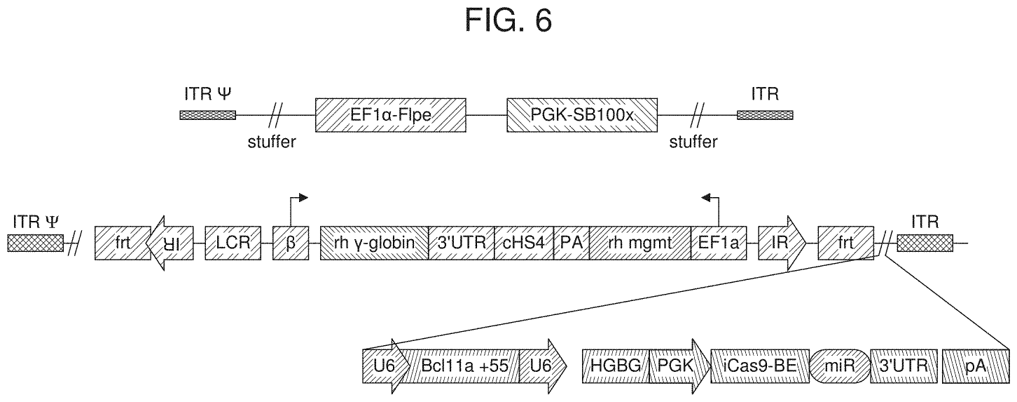

Gene transfer vector. A gene transfer vector, HDAd combination (HDAd-combo), will be used: The vector contains a SB100x transposase-mediated random genomic integration of the following transgenes: i) rhesus γ-globin gene under the control of a mini-LCR for efficient expression in red blood cells, ii) rhesus MGMT^{P140K} under control of the ubiquitously active EF1α promoter for in vivo selection of transduced cells with O⁶BG/BCNU, iii) GFP under control of the ubiquitously active EF1α promoter for analysis of peripheral blood T-cell transduction and vector biodistribution studies. It will further include adenine base editors for reactivation of endogenous γ-globin through inactivation of the BCL11a repressor protein binding sites in the HBG promoters and simultaneous inactivation of the erythroid bcl11a enhancer (which results in reduced BCL11a repressor protein expression in erythroid cells). Furthermore, the base editor expression cassette will be removed upon Flp recombinase mediated excision of the transposon resulting in only transient expression of iCas-BE. Lastly, the vector containing the SB100x transposase and Flp recombinase will not integrate and will be lost during HSC cell proliferation (FIG. 6).

Treatment protocol: The six-months study will be performed with three Macaca mulatta using HSC mobilization and O⁶BG/BCNU in vivo selection protocols (FIG. 5). The protocol will begin with testing one animal. The study will be repeated in the remaining two animals when no serious complications occur by week 8 (end of the last in vivo selection cycle).

Mobilization: There will be 5 days of GCSF and SCF given subcutaneously in the morning (50 ug/kg each). The last two days of GCSF/SCF and AMD3100 given subcutaneously will occur in the afternoon (5 mg/kg).

Pretreatment: Dexamethasone dosed at 4 mg/kg will be given intravenously 16 hours before HDAd5/35++ injection. Methylprednisolone dosed at 20 mg/kg plus dexamethasone dosed at 4 mg/kg will be given intravenously, while anakinra dosed at 100 mg will be given subcutaneously 30 minutes before HDAd5/35++ injection.

HDAd injection: Two rounds of HDAd injections will be given intravenously: (a) a low dose (3E11 vp/kg in 20 mL of phosphate buffered saline at 2 mL/min) on day −1, (b) two full doses (1E12 vp/kg in 20 mL of phosphate buffered saline at 2 mL/min) will be given 30 minutes apart at day 0.

Transient immunosuppression: Immunosuppression will begin starting at day 1 until the first dose of O⁶BG/BCNU (week 4), and if required, continued 2 weeks after the last dose of O⁶BG/BCNU. The immunosuppression will include 0.2 mg/kg/day of rapamycin, 30 mg/kg/day of mycophenolate mofetil, and 0.25 mg/kg/day of tacrolimus, all given daily, orally via food.

In vivo selection with O⁶BG/BCNU: O⁶BG: Animals will receive 120 mg/m² O⁶BG in 200 mL of saline, intravenously infused over at least 30 minutes. BCNU will be administered 60 minutes after the start of O⁶BG infusion. Animals will then receive another dose of O⁶BG in 200 mL of saline intravenously over at least 30 minutes six to eight hours after BCNU administration. The first treatment will be given four weeks after HDAd injection; the second and third treatment with 2 weeks intervals (optional), depending on γ-globin marking and hematology.

Data to be collected: Blood samples will be collected as indicated in FIG. 5. Daily physical observation and weekly body weight measurements will be performed.

Blood samples: For two- and six-hour blood samples, the following assays will be performed: percentage of GFP+ cells in CD34+ and percent of GFP+ cells in CD38₋/Cd45RA, CD90+ cells will be quantified, colony forming unit assays will be used to assess percent of % GFP+ colonies, migrations towards SDF1-a, and percent expression of CXCR4 and/or VLA-4. For all other samples, blood cell counts, chemistry, c-reactive protein, and proinflammatory cytokines will be measured. γ-globin expression will be measured via flow cytometry (erythroid/non-erythroid cells), while HPLC and qRT-PCR will be used to measure levels of re-activated vs added γ-globin. Cytospins will be used to assess γ-globin immunofluorescence. Vector copy number and Cas9, SB100x, and Flpe mRNA levels will be measured. GFP expression in white blood cells (CD4+, CD8+, CD25, CD45RO, CD45RA, CCR-7, CD62L, FOXP3, integrin αeβ7) will be measured.

Bone marrow samples: Bone marrow samples will be collected on day four and then monthly (see FIG. 5). Lineage composition of bone marrow samples will be assessed by flow cytometry. Vector copy numbers in CD34+ cells will also be measured. γ-globin will be assessed using flow cytometry by sorting with Ter119+/Ter119− markers. HPLC and qRT-PCR will be used to measure levels of re-activated vs added γ-globin. In addition to these analyses, upon necropsy, whole genome sequencing will be performed on CD34+ cells to identify SB100x-mediated integrations and base editor off-target effects. RNA sequencing will also be performed on CD34+ cells to compare mRNA and miRNA profiles between pre- and post-treatment.

Tissues from necropsy (including germline tissues and semen): Routine histology will be performed, and vector copy numbers will be measured on major tissue groups. γ-globin and GFP immunofluorescence will be assess on tissue sections.

Outcome: This experiment will validate that both the SB100x-mediated gene addition and the BE-mediated reactivation of endogenous γ-globin are effective in non-human primates after in vivo HSC transduction. It will demonstrate that the vector will achieve γ-globin expression levels in red blood cells that would be curative in SCA patients (i.e., >80% γ-globin+ RBCs with γ-globin levels >20% of adult rhesus globin). It will also demonstrate an absence of long-term hematological side-effects and absence of undesired genomic rearrangements and changes in the transcriptome of HSCs. Lastly, it will demonstrate that intravenously injected HDAd5/35++ vector transduces memory T-cells.

### Example 5: In Vivo HSC Gene Therapy for Hemoglobinopathies: In Vivo Gene Therapy in Rhesus Macaques Including a Viral Vector, a Support Vector, an Immune Suppression Regimen, a Selecting Agent, and a Stem Cell Mobilization Regimen

Many gene therapy or genome editing studies for hemoglobinopathies require highly sophisticated medical facilities to perform hematopoietic stem cell collections/selections and genetic modifications. In addition, patients receive high-dose chemotherapy to facilitate engraftment of gene-modified cells. Thus, certain gene therapy protocols are inaccessible to many patients suffering from hemoglobinopathies. Certain of the material in this Example was published as Li et al. (Blood, 136(Supp. 1): 46-47, 2020; doi.org/10.1182/blood-2020-141468).

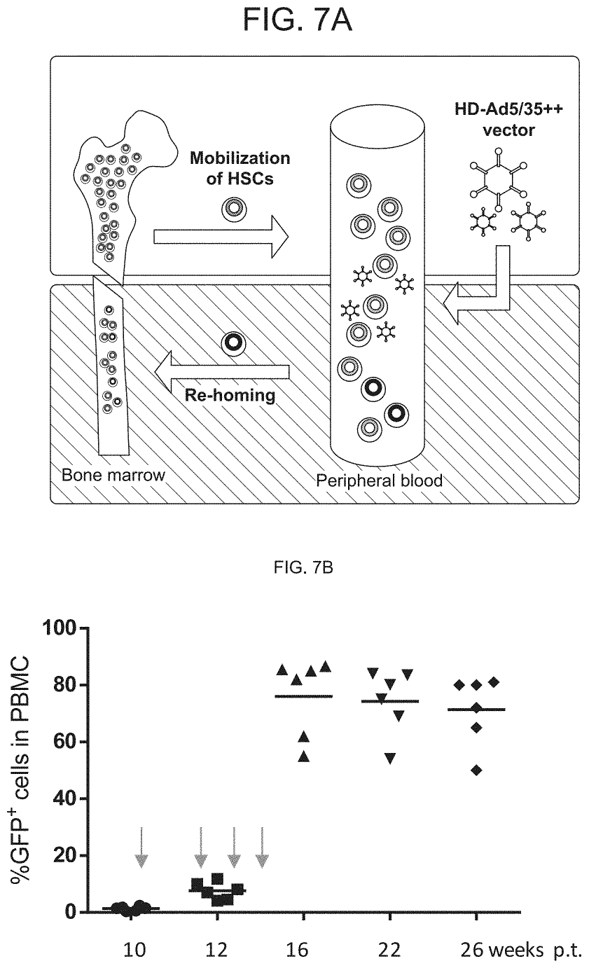

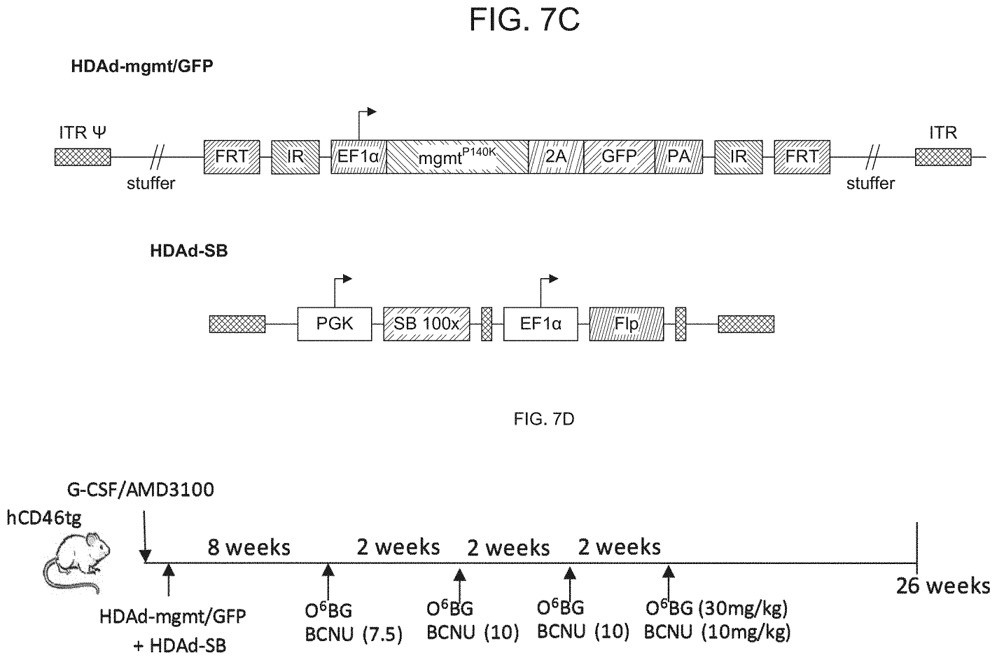

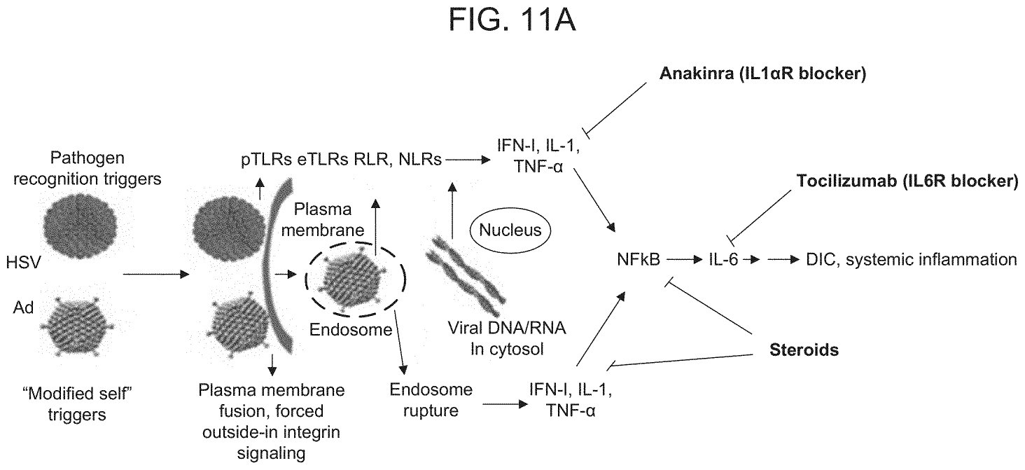

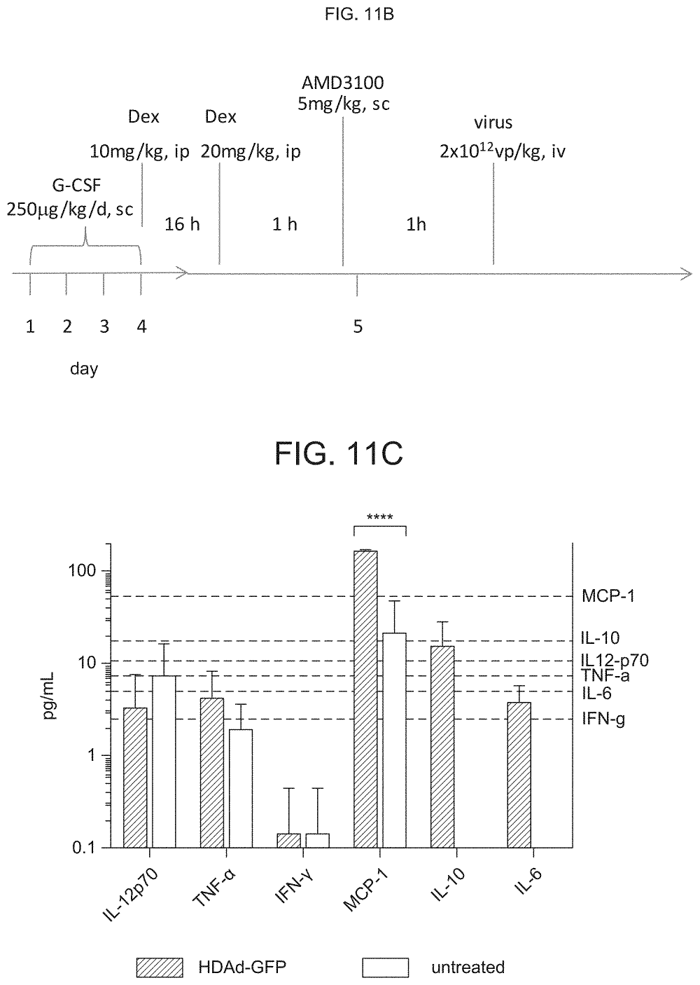

The present Example includes a highly portable and scalable gene therapy approach that includes in vivo hematopoietic stem cell (HSC) gene therapy and potentially overcomes these limitations. In the present in vivo HSC gene therapy approach, HSCs are mobilized from the bone marrow and, while they circulate at high numbers in the periphery, are transduced with an intravenously injected HSC-tropic, helper-dependent adenovirus HDAd5/35++ gene therapy vector system (see schematic of FIG. 7A). Transduced cells return to the bone marrow where they persist long-term (see exemplary illustration in FIG. 7B). Integration of a therapeutic payload encoded by the gene therapy vector can be achieved by a Sleeping Beauty transposase (SB100x) in a random pattern or by homology directed-repair into a safe genomic harbor site. In the present Example, vectors including payloads that include a selectable marker of an in vivo selection system (mgmt^{P140K} selectable marker, with selection by low-dose O⁶BG/BCNU) are employed to achieve 80-100% marking levels in peripheral blood cells. Safety and efficacy of HDAd5/35++ vectors has been demonstrated in mouse models for thalassemia intermedia, Sickle Cell Disease, and hemophilia A, where a phenotypic correction was achieved. It has been observed that administration of adenoviral vectors induces an innate immune response (for example, see schematic in FIG. 11A, which further shows certain immune suppression agents of the present disclosure). Observations in mice support the possibility that administration of an immune suppression regimen can blunt the immune response to adenoviral vector administration (FIGS. 11B and 11C).

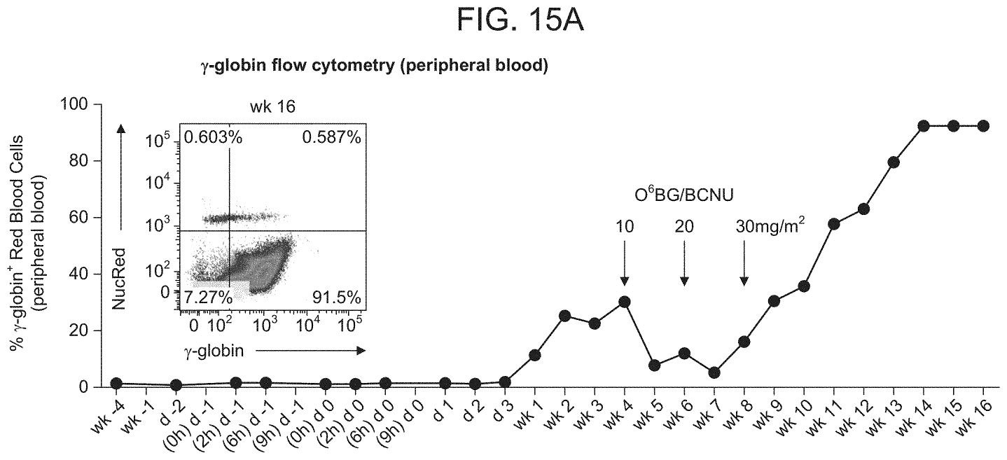

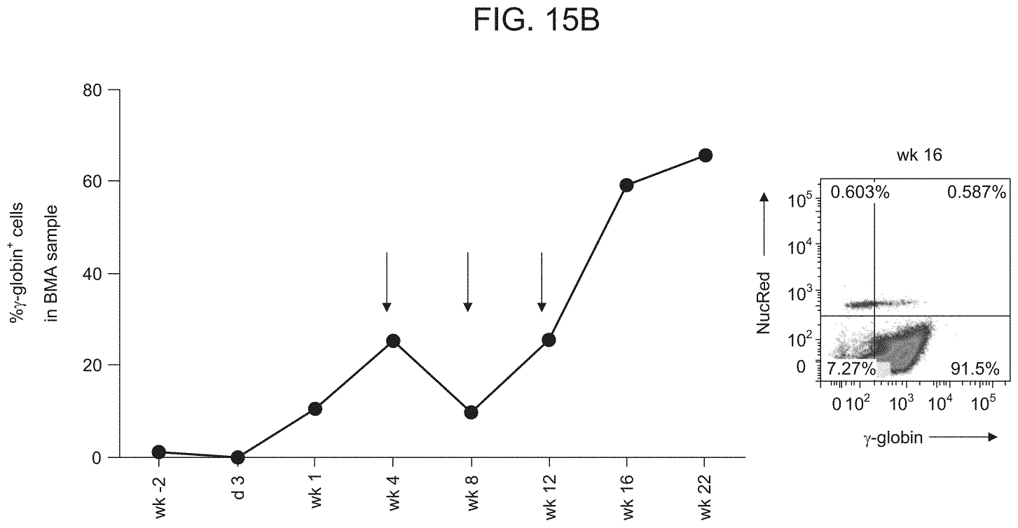

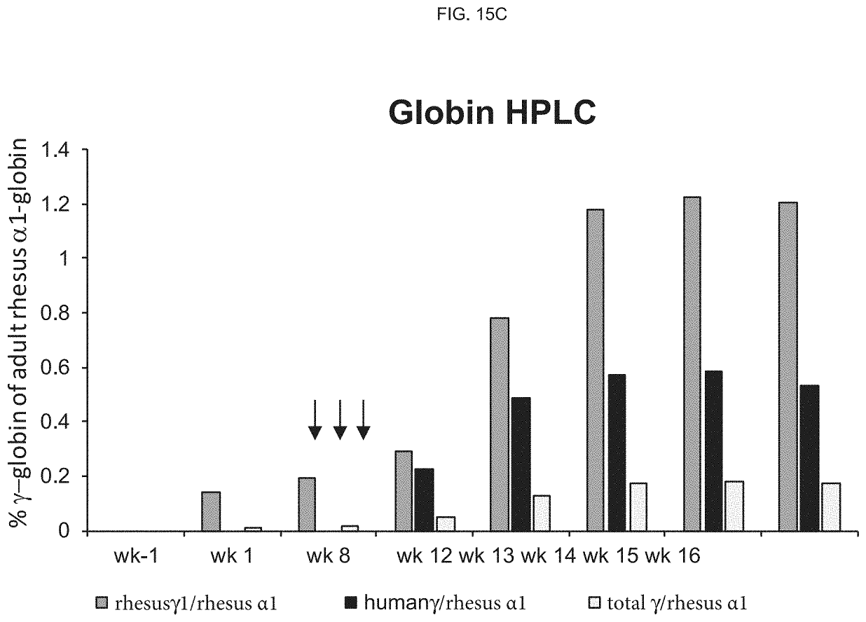

The present Example shows that a new immune suppression regimen (dexamethasone, IL-6 receptor antagonist, IL-1 receptor antagonist, saline bolus IV) was able to mitigate side effects associated with intravenous HDAd5/35++ vector administration. Data in 3 rhesus macaques is presented. It is shown that treatment with G-CSF/AMD3100 resulted in efficient HSC mobilization into the blood circulation and that subsequent intravenous injection of the HDAd5/35++ vector system (total 1-3×10¹² vp/kg, in two doses) was well tolerated. After in vivo selection with O⁶BG plus low dose (10 to 20 mg/m²) of BCNU, a dose that is up to 100-fold lower than the dose used in certain autologous transplantation protocols, gamma-globin marking in peripheral red blood cells rose to 90% and was stable for the duration of the study (see FIG. 15A). Gamma-globin levels in red blood cells were 18% of adult alpha1-globin (by HPLC). Analysis of day 3 bone marrow showed 30% transduced HSCs. Vector DNA biodistribution studies demonstrated very low or absent transduction of most tissues (including testes and CNS). Analysis of bone marrow showed efficient, preferential HSC transduction and re-homing of transduced CD34+/CD90+ cells to the bone marrow. At week 4, about 5% of progenitor colony-forming cells demonstrated stable transduction with integrated vector, and this frequency increased after starting the in vivo selection. The level of human mgmt^{P140K} mRNA expression in PBMCs also increased after in vivo selection.

### Summary of Results

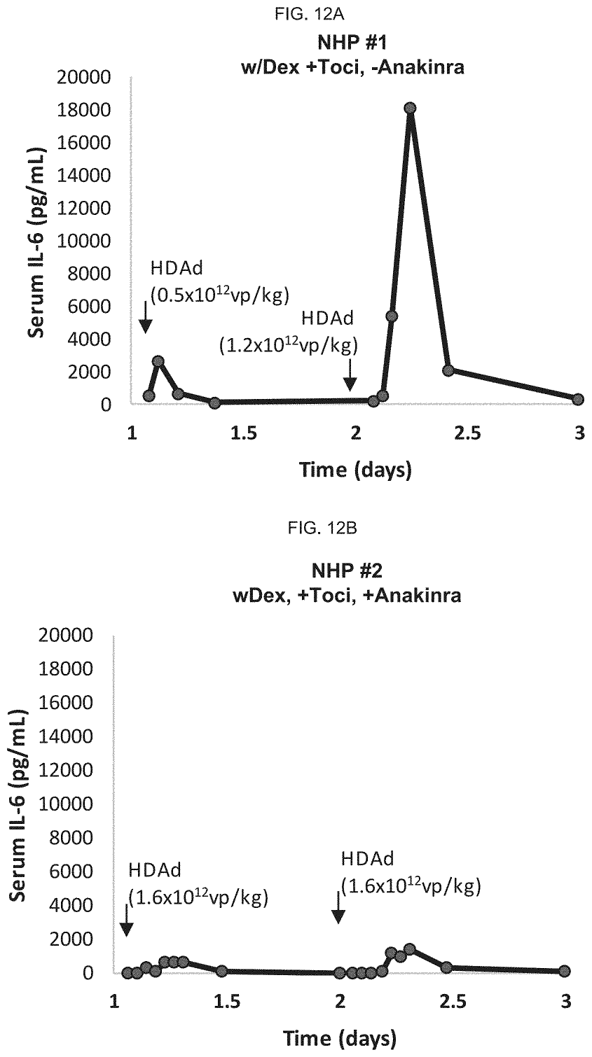

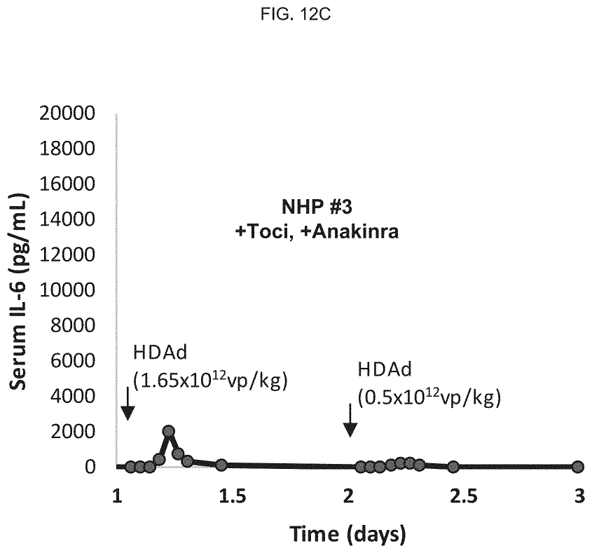

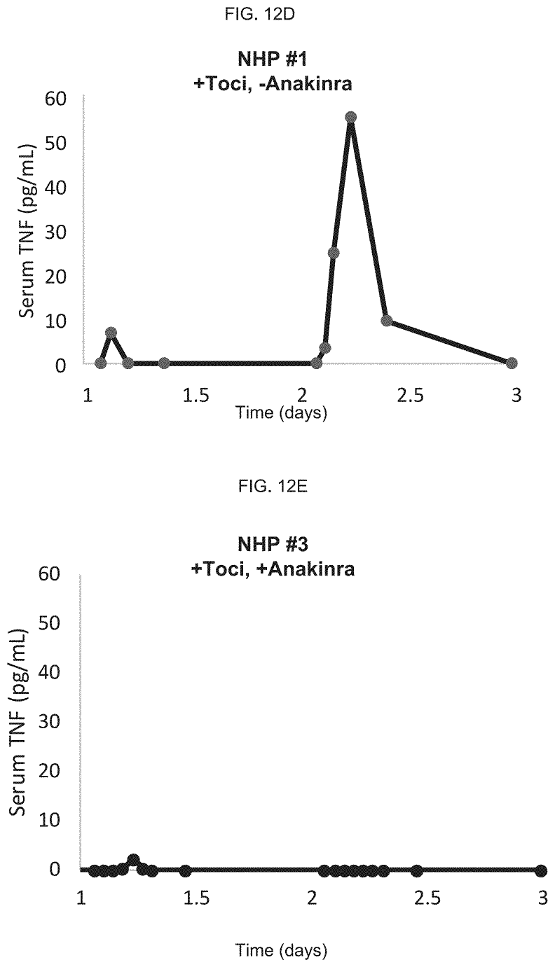

Using a new and optimized immune suppression regimen, intravenous delivery of adenoviral vector (exemplified by HDAd5/35++) was very well tolerated without significant detected cytokine activation. As shown in FIGS. 12A-12C, an NHP treated with an immunosuppression regimen including an IL-6 signal inhibitor (tocilizumab) and a corticosteroid (dexamethasone) experience a robust immune response (measured by serum IL-6) upon adenoviral vector administration, while NHPs receiving an immunosuppression regimen including an IL-6 signal inhibitor (tocilizumab), an IL-1 signal inhibitor (anakinra), and a corticosteroid (dexamethasone) demonstrate little detectable immune response by the same measure. Moreover, combination of anakinra with tocilizumab and dexamethasone, but not tocilizumab and dexamethasone alone, abrogated the immune response to adenoviral vector administration as measured by serum TNFα (FIGS. 12D and 12E). These results together demonstrate the unexpectedly robust regulation of the immune response(s) to adenoviral vector administration by anakinra and regimens including anakinra. Efficient transduction of HSCs and efficient in vivo selection of transduced progenitors with low dose O⁶BG/BCNU were demonstrated. To the knowledge of the present inventors, the present Example is the first demonstration that in vivo HSC gene therapy is feasible in humans without the need for high-dose chemotherapy conditioning and without the need for highly specialized medical facilities. This approach provides a major advance for the gene therapy and genome editing fields and enables the necessary portability and accessibility to reach patients in places with limited medical resources.

### Summary of Methods

### Vector Used: HDAd5/35++

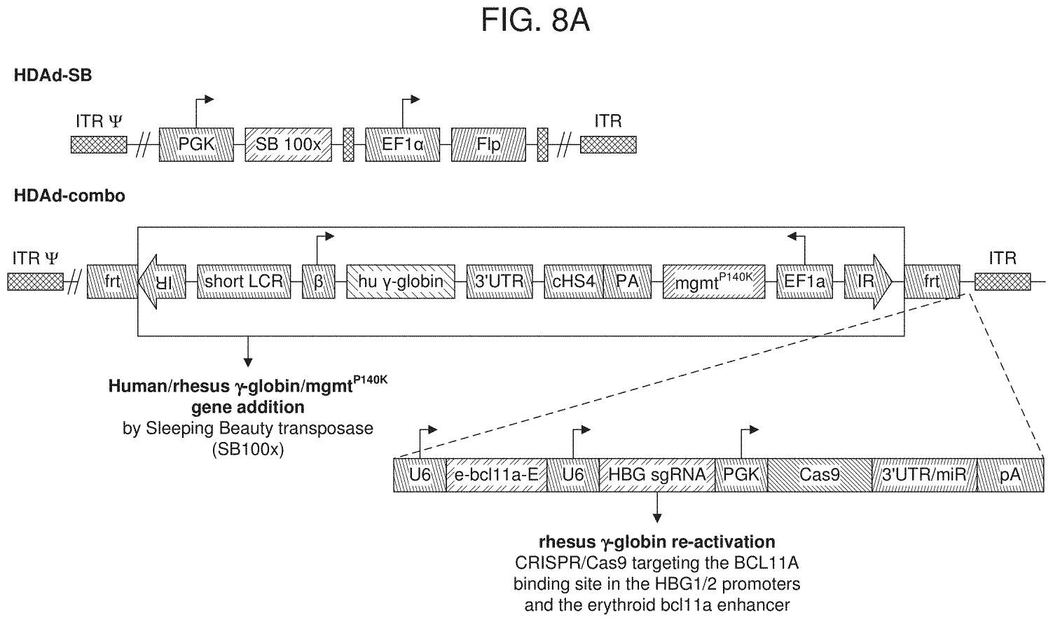

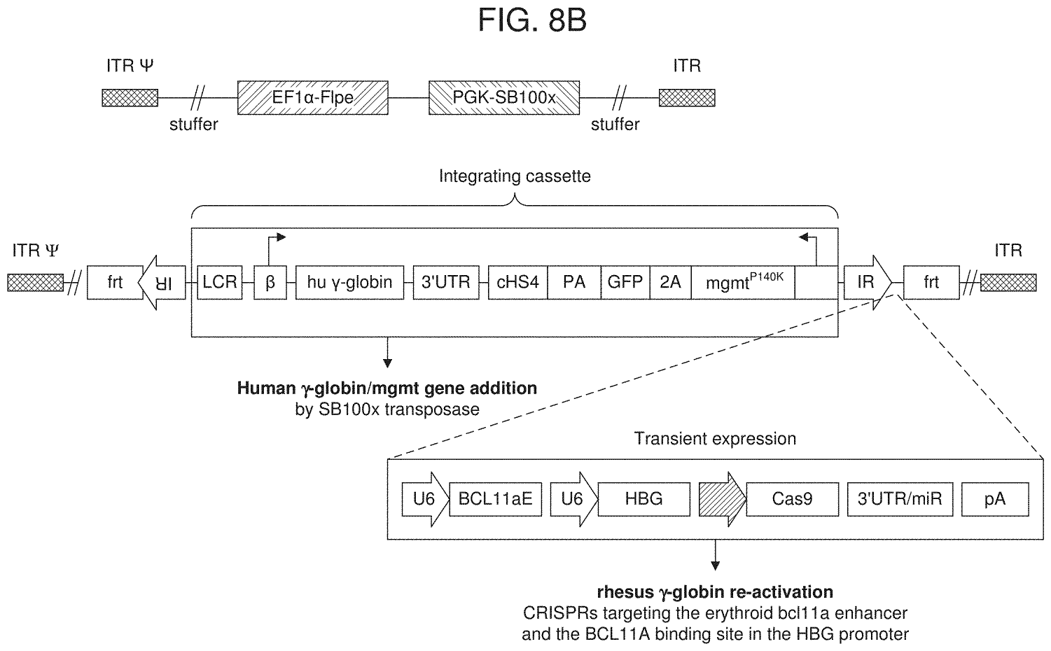

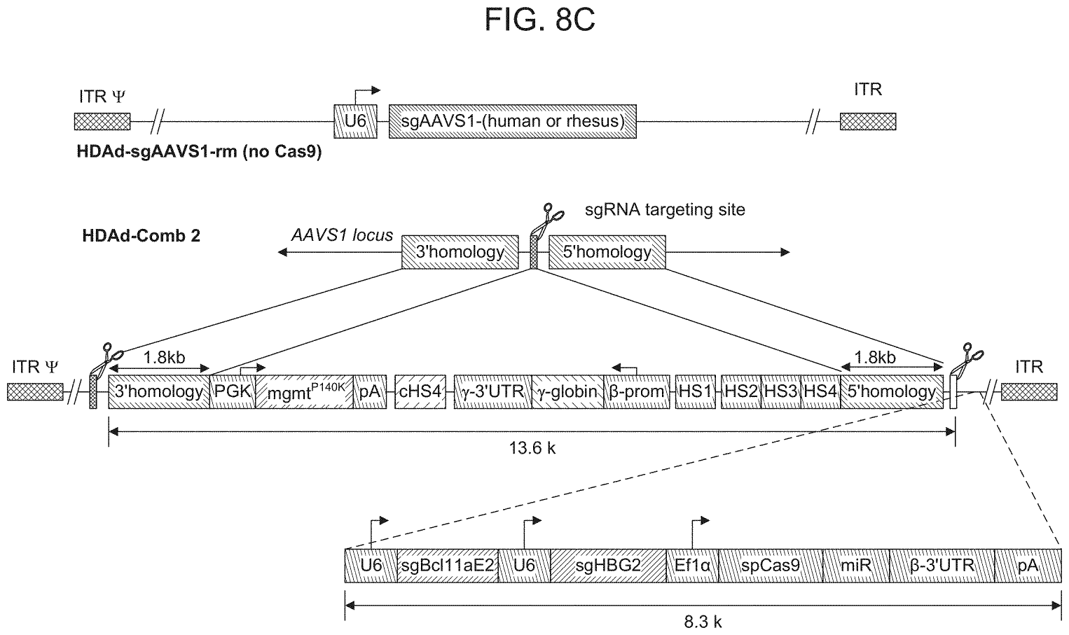

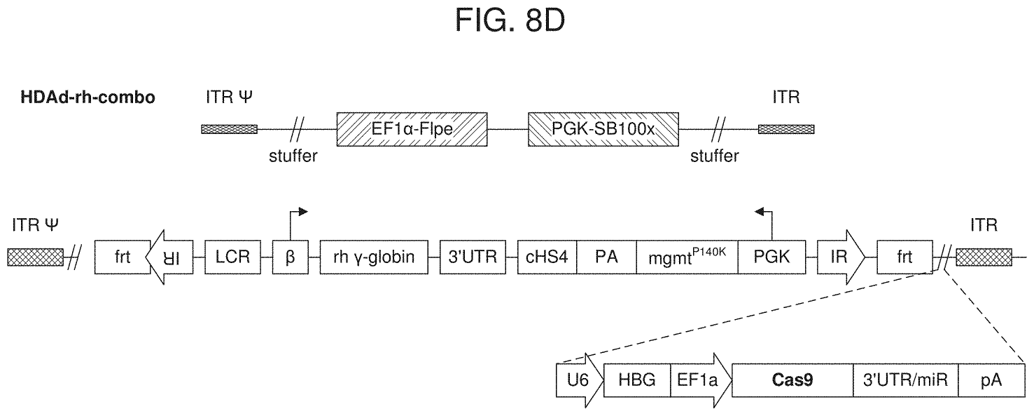

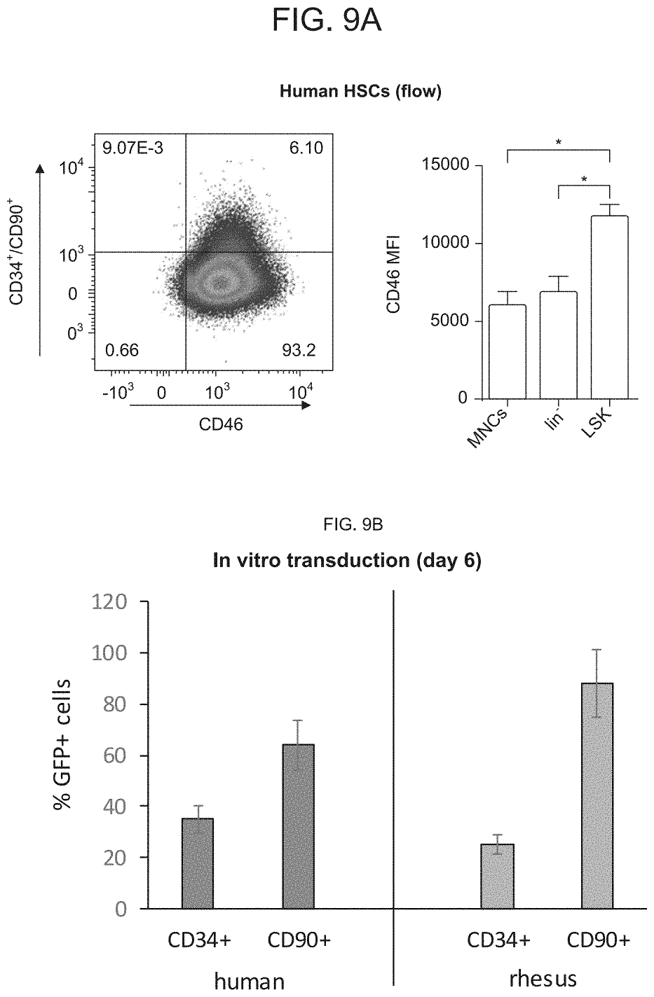

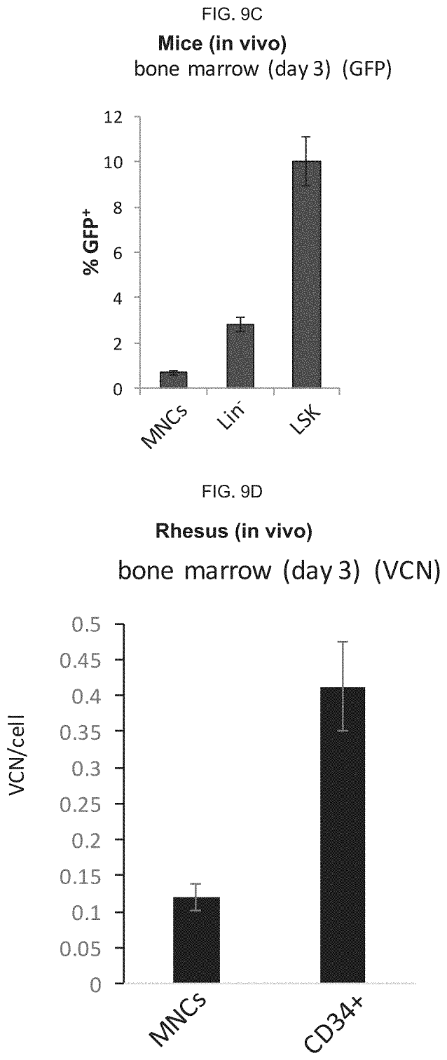

Vector payloads: Administered vector included a 1:1 mixture of HDAd5/35++ donor vector and HDAd5/35++ support vector, i.e. in which the donor vector included a transposon and the support vector encoded transposon integration machinery (see exemplary illustration of donor and support vectors in FIGS. 7C and 8A-8D). In particular examples provided herein, the support vector encoded SB100x transposase and Flpe recombinase and the donor vector encoded a transposon flanked by IRs for SB recognition and FRT sites for Flpe recombinase recognition. In certain examples provided herein, the transposon included human gamma globin cDNA operably linked with a β-globin promoter/LCR and a GFP/MGMT^{P140K} cassette operably inked with a PGK promoter (see schematic of an exemplary selection protocol in FIG. 7D). In certain examples, donor vector payload further included, outside of the transposon, a rhesus gamma globin re-activation CRISPR/Cas9 cassette encoding guide sequences targeting the erythroid bcl11a enhancer and the BCL11A binding site in the HBG promoter, each under control of a U6 promoter, and spCas9 cDNA under control of the EF1α promoter. In certain examples, gamma globin reactivation cassette was inserted outside of the 3′ IRs (for SB recognition) and frt sites (for Flp-mediated recombination) allowing for transient expression of the reactivation cassette. HDAd5/35++ vectors preferentially transduce HSCs (see exemplary data in FIGS. 9A-9D).

The HDAd5/35++ vectors were administered in two infusions (each over 40 min) 24 h apart. The first infusion (on day −1) was between 0.5-1.65E12 vp/kg; the second HDAd5/35++ infusion (on Day 0) was between 0.5-1.6E12 vp/kg.

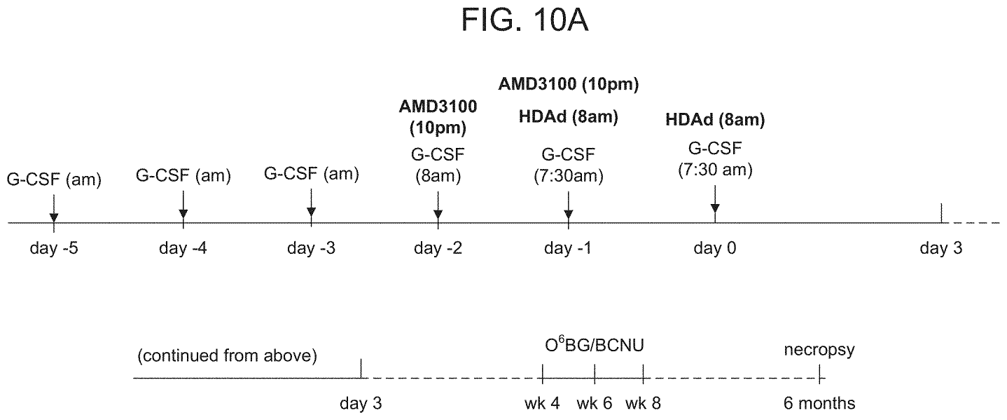

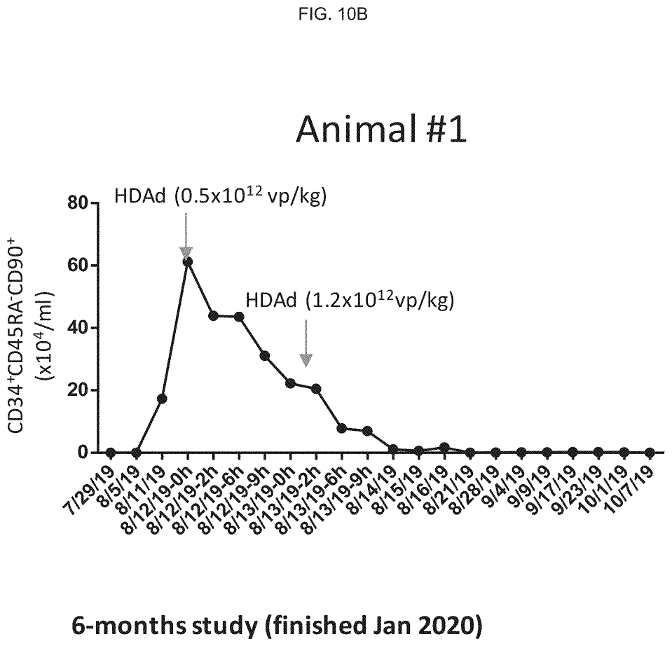

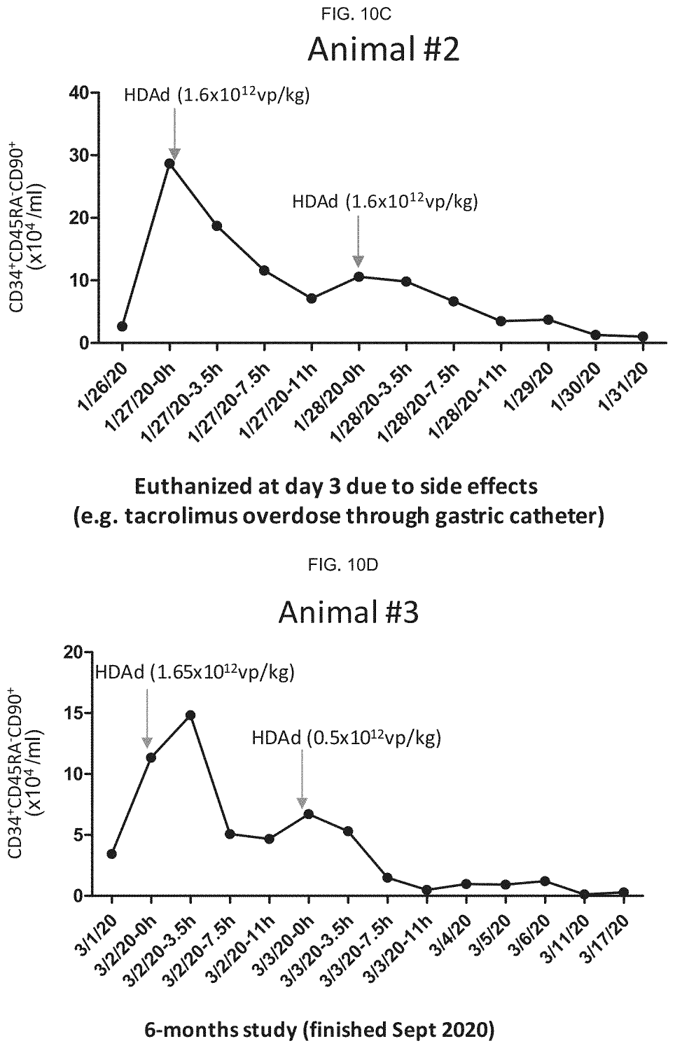

Mobilization regimen: Starting on day −5 and continuing through Day 0 (six days of treatment) each animal received a SQ injection of G-CSF at a dose of 50 pg/kg. AMD3100 (5.0 mg/kg) was given SQ twice, the first injection on day −2 (10 PM) and the second injection on day −1 (10 PM) (FIG. 10A). Mobilization was effective all three NHPs (FIGS. 10B, 10C, and 10D).

Immune suppression regimen: Starting on Day −2 and continuing through day 0, the animals received an IV dose of dexamethasone (5.0 mg/kg). This was supplemented with either Actemra® (tocilizumab) alone (NHP #1) given IV at a dose of 8.0 mg/kg, starting on Day −1 and continuing through Day 2, or Actemra® with Anakinra (IV 50 mg/animal, NHP #2 and NHP #3) starting on Day −1 and continuing through Day 2.

TABLE 4
Immune suppression regimen

| | Immune Suppression Agents Administered, and Doses | | |
|---|---|---|---|
| Day | NHP#1 | NHP#2 | NHP#3 |
| −6 | | | |
| −5 | | | |
| −4 | | | |
| −3 | | | |
| −2 | dexamethasone | dexamethasone | dexamethasone |
| | (2.0 mg/kg) qd | (7.0 mg/kg) qd | (4.0 mg/kg) qd |
| −1* | dexamethasone | dexamethasone | dexamethasone |
| | (2.0 mg/kg) bid | (7.0 mg/kg) bid | (4.0 mg/kg) bid |
| | Prednisolone | tocilizumab | tocilizumab |
| | (10 mg/kg) bid | (8.0 mg/kg) bid | (8.0 mg/kg) bid |
| | tocilizumab | anakinra | anakinra |
| | (8.0 mg/kg) bid | (50 mg/animal) bid | (50 mg/animal) bid |
| 0* | dexamethasone | dexamethasone | dexamethasone |
| | (2.0 mg/kg) bid | (7.0 mg/kg) bid | (4.0 mg/kg) bid |
| | Prednisolone | tocilizumab | tocilizumab |
| | (10 mg/kg) bid | (8.0 mg/kg) bid | (8.0 mg/kg) bid |
| | Tocilizumab | anakinra | anakinra |
| | (8.0 mg/kg) bid | (50 mg/animal) bid | (50 mg/animal) bid |
| 1 | | | tocilizumab |
| | | | (8.0 mg/kg) qd |
| | | | anakinra |
| | | | (50 mg/animal) qd |
| 2 | | | tocilizumab |
| | | | (8.0 mg/kg) qd |
| | | | anakinra |
| | | | (50 mg/animal) qd |

*Days on which adenoviral vector was administered.

Selection regimen: On Day 28 and on Day 57, each animal received an IV infusion of BCNU (20 mg/m²) and O⁶BG (120 mg/m2) given over 30 min.

### HDAd5/35++ Vectors and NHP Study Overview:

Three male rhesus macaques were treated in these studies with N=1 per HDAd5/35++ vector used. NHP #1 received a donor vector containing two payload modules: i) a transposon module for random integration into host cell genomes of a human γ-globin gene operably linked with a mini-β-globin LCR, together with an MGMT^{P140K} selection marker operably linked with an EF1α promoter, and ii) a CRISPR/Cas9 module for CRISPR/Cas9-mediated reactivation of endogenous rhesus γ-globin expression (FIG. 8B). This combination donor vector was co-injected with a support vector that expresses an activity-enhanced Sleeping Beauty transposase (SB100x) (HDAd-SB), activity of which mediates γ-globin gene addition by integration of the transposon into host cell genomes and triggers the destruction of the CRISPR/Cas9 module thus restricting the duration of the CRISPR/Cas9 system's expression and activity (FIG. 8B). NHP #2 was injected with a HDAd5/35++ donor vector for targeted integration of a γ-globin cassette into a safe genomic harbor, the AAVS1 locus (FIG. 8C). The vector injected into NHP #3 was similar to the NHP #1 vector but encoded rhesus γ-globin instead of human γ-globin to minimize immune responses against transgene products in the NHP (FIG. 8D).

None of the three animals had detectable anti-vector antibodies at the time of enrollment. Unexpectedly, NHP #1 acquired antibodies during quarantine (titer 1:680). NHP #1 and #3 were monitored for 6 months. NHP #2 had to be euthanized on day 3 after HDAd injection due to a tacrolimus overdose (given through a gastric catheter over 5 days). Despite this, a set of relevant data could be collected from this animal. For in vivo HSC selection, NHP #1 received 30 mg/kg O⁶BG plus 10, 20, and 30 mg/kg BCNU on weeks 4, 6, and 8, respectively. NHP #3 was treated with 30 mg/kg O⁶BG plus 10, 20, and 20 mg/kg BCNU on weeks 4, 8, and 13.

HSC Mobilization. HSC were mobilized by four injections of G-CSF (SC, AM) followed by AMD3100 (SC PM) given 11 hours before HDAd injection. This timing considers that in humans, the peak of mobilized HSCs is 11 hours after AMD3100 administration. As outlined below, unlike humans, NHPs express CD46 (the target receptor for HDAd5/35++ vectors) on red blood cells. This bears the risk of sequestration of injected HDAd5/35++ particles. To address this, the mobilization regimen was modified and a second injection of HDAd5/35++ was given (FIGS. 10A-10D).

Immune suppression: A hallmark of the innate immune activation resulting from exposure to adenoviral vectors is the elevation of proinflammatory cytokines. In particular, IL-1 and IL-6 signaling appear to be critically important in mediating the adverse effects with systemically administered adenoviral vectors (Shayakhmetov et al., J Immunol. 174(11): 7310-7319, 2005; Koizumi et al., J Immunol. 178(3): 1767-1773, 2007; Benihoud et al., J Gene Med. 2(3): 194-203, 2000). Regimens to minimize innate (cytokine) responses and adaptive immune responses against human transgene products (MGMT^{P140K} and γ-globin) evolved during the studies (Table 5). NHP #1 was administered with prophylactic immune suppression regimen including dexamethasone (2 mg/kg) and tocilizumab (8 mg/kg). This regimen was not sufficient to completely suppress the release of IL-6 and TNF-α which peaked at 6 hours after vector dosing (FIGS. 12A-12E). Serum cytokines returned back to baseline by 24 hrs. A new immune suppression regimen (dexamethasone, IL-6R antagonist, IL-1bR antagonist, saline bolus IV) mitigated all side effects associated with intravenous HDAd5/35++ vector administration. Including the IV fluid bolus prevented hypotension and subsequent nausea.

TABLE 5
Immune suppression

| | Vector | Cytokine | Additional | Study | |
|---|---|---|---|---|---|
| Animal | doses | prophylaxis | Immunosuppression | duration | |
| NHP#1 | 0.5 and | Dexamethasone, | Tacrolimus, MMF, | 148 | days |
| (11.5 kg) | 1.2 × 10¹² vp/kg | tocilizumab | sirolimus (IM) | | |
| NHP#2 | 1.6 and | Dexamethasone, | Tacrolimus (gastric | 6 | days |
| (9.0 kg) | 1.6 × 10¹² vp/kg | tocilizumab, | catheter) | | |
| | | anakinra | | | |
| NHP#3 | 1.6 and | Dexamethasone, | Tacrolimus, MMF (IM), | 192 | days |
| (6.0 kg) | 0.5 × 10¹² vp/kg | tocilizumab, | abatacept (IV wk 21- | | |
| | | anakinra | wk 24) | | |

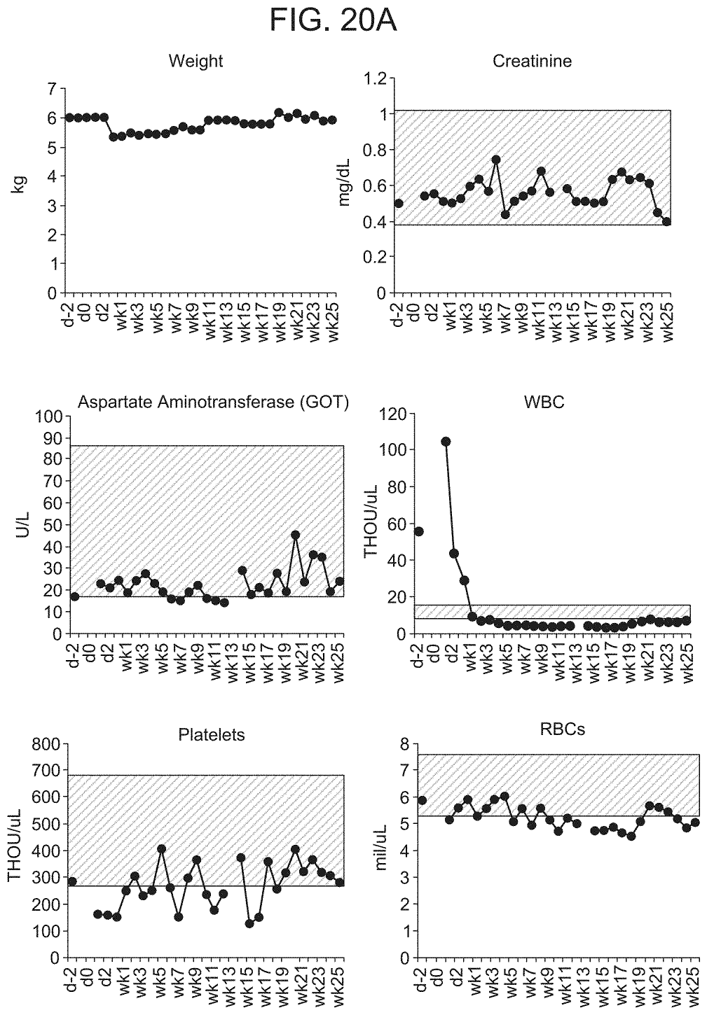

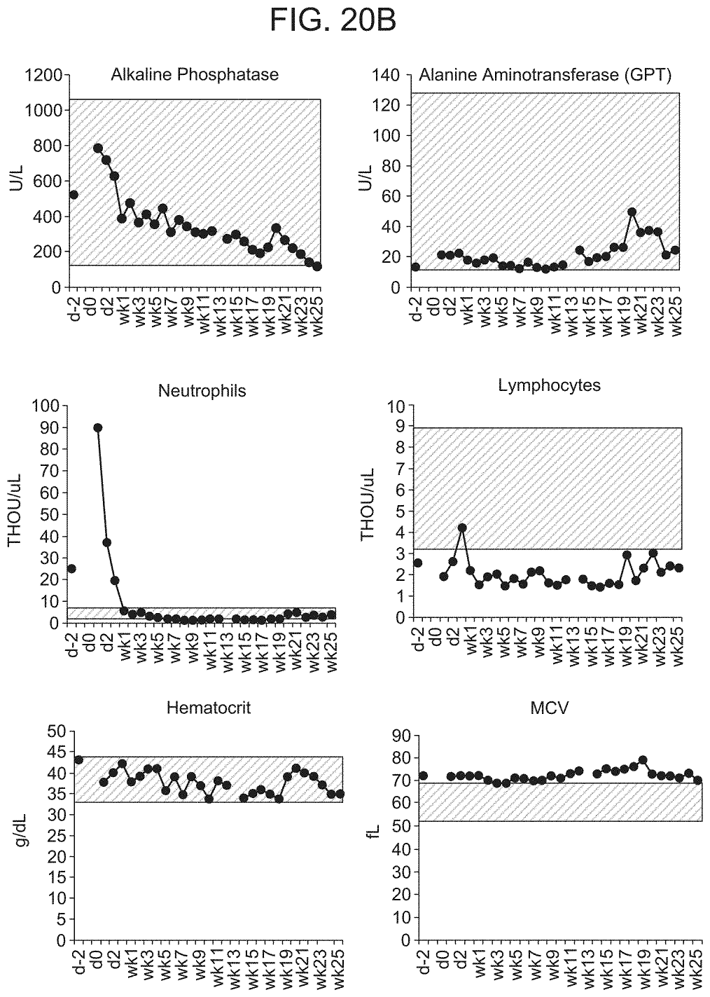

Notably, GCSF/AMD3100 mobilization resulted in a critical increase in neutrophil counts from day 0 to day 4 in all three animals (see also FIG. 20). Neutrophils contain pro-inflammatory cytokines that can be released during senescence. The immune suppression regimen administered to NHP #3 aimed to counteract this by including anakinra/tocilizumab also on days 1 and 2.

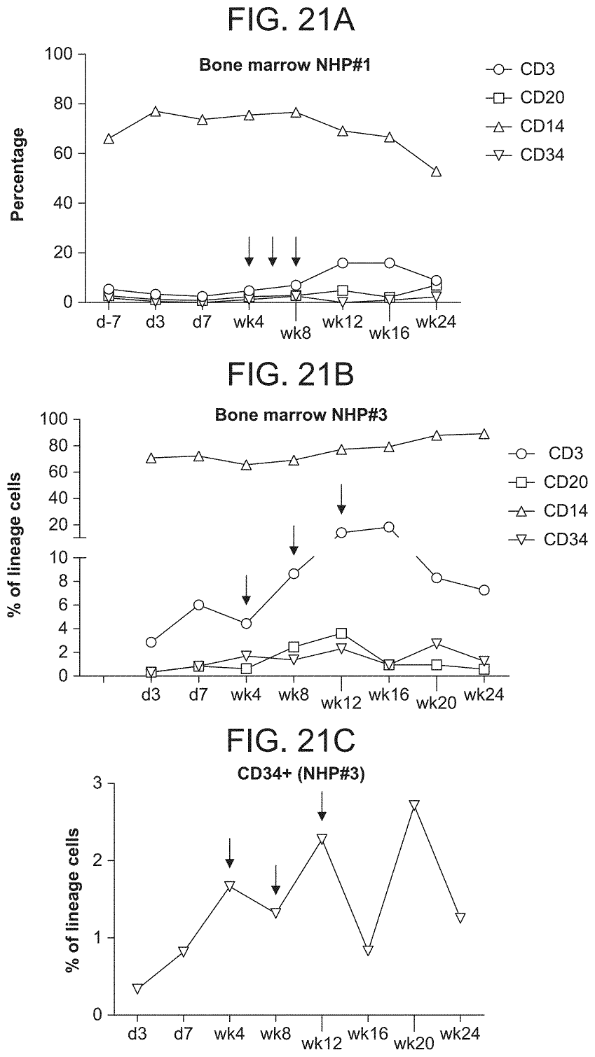

To suppress adaptive immune responses, NHP #1 received daily tacrolimus/sirolimus/MMF. Given over a longer time period, this regimen caused GI-tract and kidney toxicity. For NHP #3, it was therefore decided to administer only tacrolimus (SC) as this had been sufficient in myelo-ablated/-conditioned rhesus macaques. However, the present study included the observation that because animals in this study were fully immunocompetent, tacrolimus alone did not prevent the development of anti-human MGMT^{P140K} antibodies (and T-cell responses) (see also FIGS. 21A, 21B). Attempts to increase immunosuppression in NHP #3 by additional treatment with MMF and CTLA4-Ig (Abatacept; Orencia®) were only partially effective. Immunosuppressive drugs given through a gastric catheter can cause severe side effects.

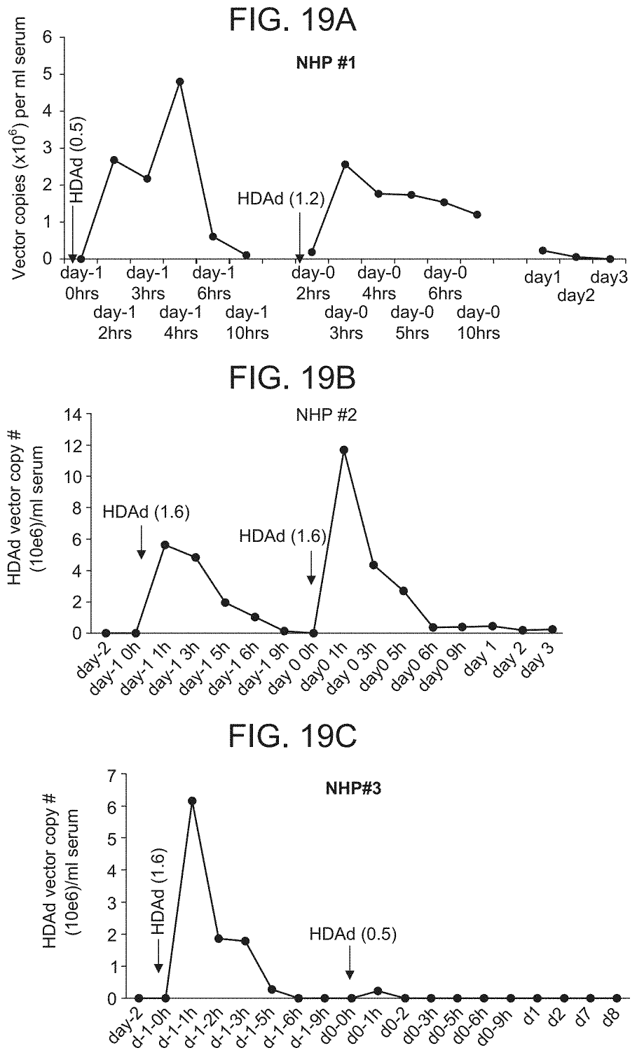

Vector serum clearance: Clearance of vector from blood after IV injection of 1.6×10¹²vp/cell showed a similar kinetics for NHP #2 and NHP #3 with a half-life of 2-3 hours (FIGS. 19A-19C). Vector was not detectable anymore by 9-10 hours after injection. In NHP #1, vector genomes were still detectable 24 hrs after injection. Notably, NHP #1 had acquired anti-HDAd5/35++ IgG antibodies during the quarantine, which could have led to the formation of immune complexes and influenced vector clearance. Overall, clearance of HDAd5/35++ appeared to be much slower than e.g. clearance of Ad5 vectors (Seshidhar et al., Virology. 311(2): 384-393, 2003).

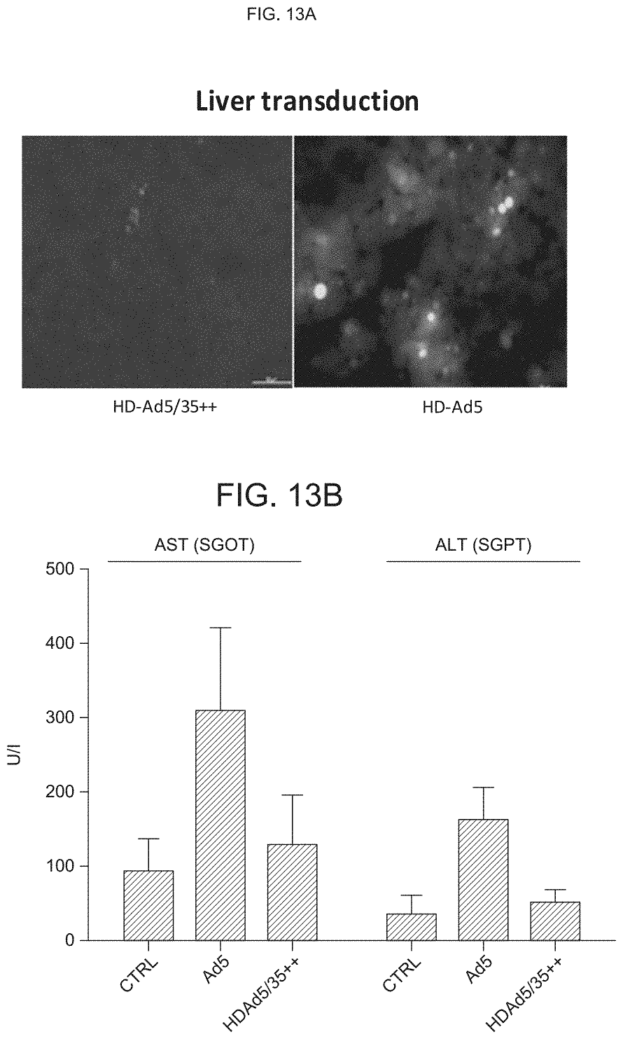

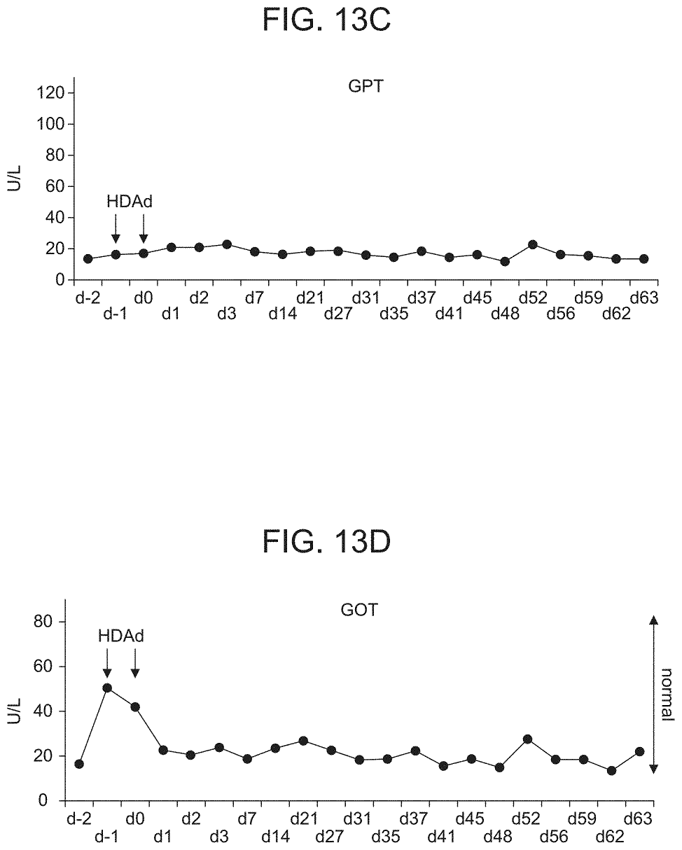

Physical health and hematology: While adverse side effects were observed in NHP #1 due to aggressive immunosuppression (tacrolimus+sirolimus+MMF) and in vivo selection (last BCNU dose: 30 mg/kg), the safety profile of NHP #3 was excellent after protocol adjustment (tacolimus only, max BCNU dose: 20 mg/kg) (FIGS. 20A-20B). No clinical signs, no unusual weight loss, no abnormal blood cell counts, and no abnormal blood chemistry were observed. Notably, liver transaminases were not elevated, in stark contrast to what would be expected for Ad5 vector injected animals (Seshidhar et al., Virology. 311(2): 384-393, 2003) (see FIGS. 13A-13D). (The HDAd5/35++ vector was designed to avoid hepatocyte uptake.) Furthermore, analysis of lineage-positive cells and CD34+ cells in the bone marrow demonstrated that the O⁶BG/BCNU doses used did not affect the bone marrow and HSCs in NHP #3 (FIG. 21A-21C).

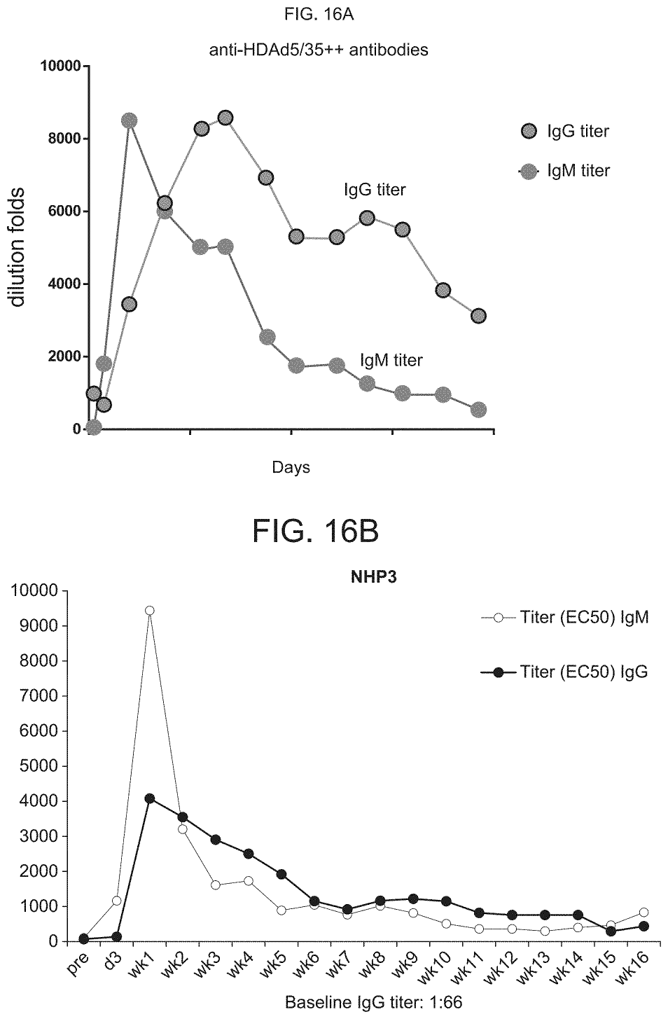

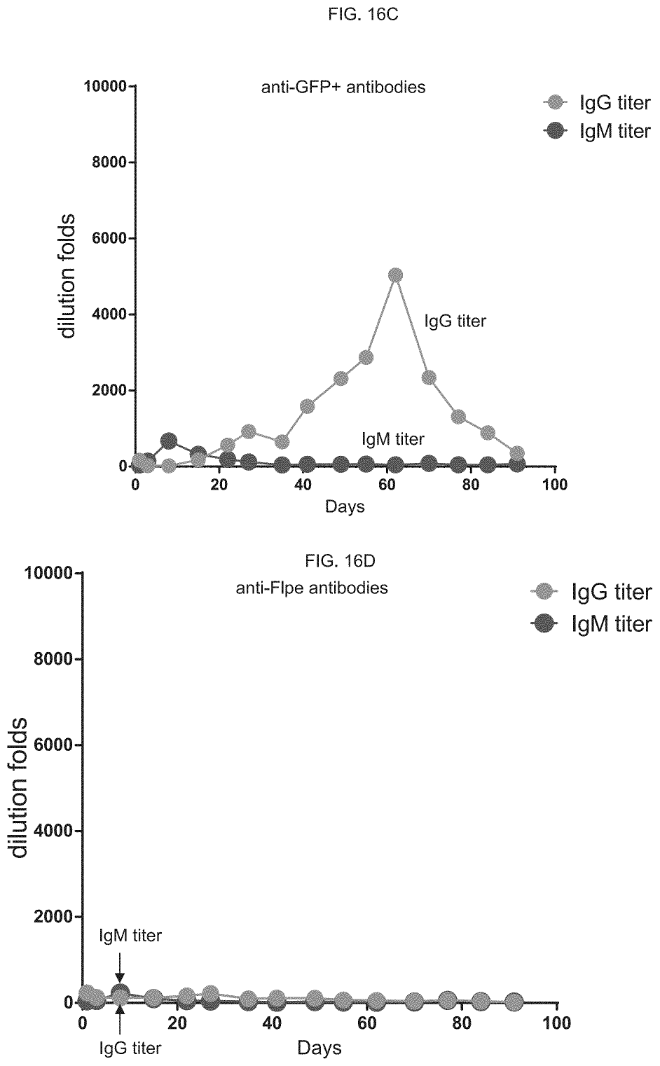

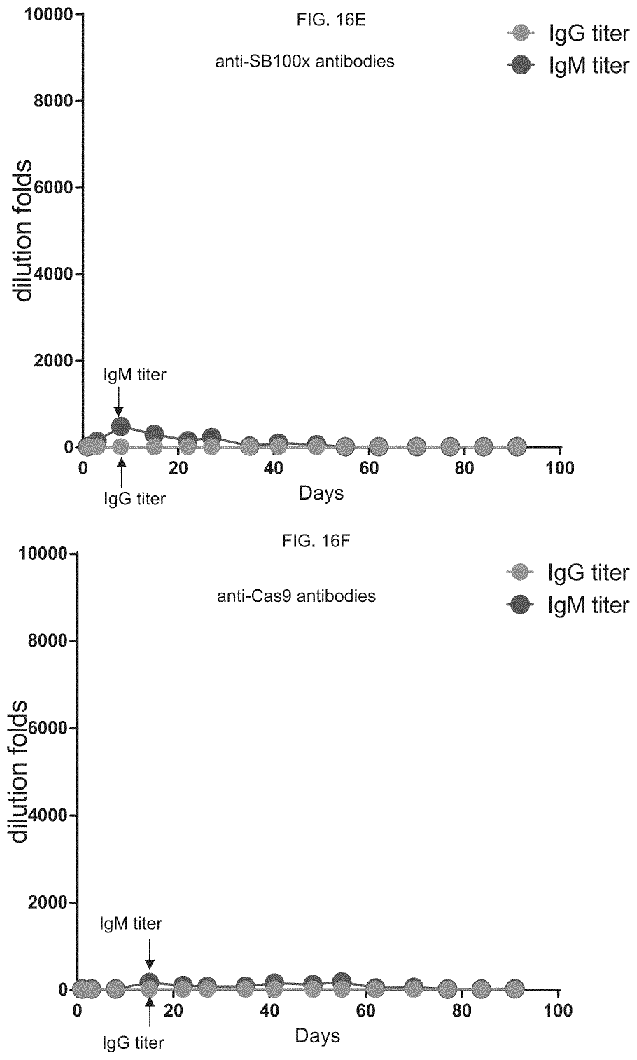

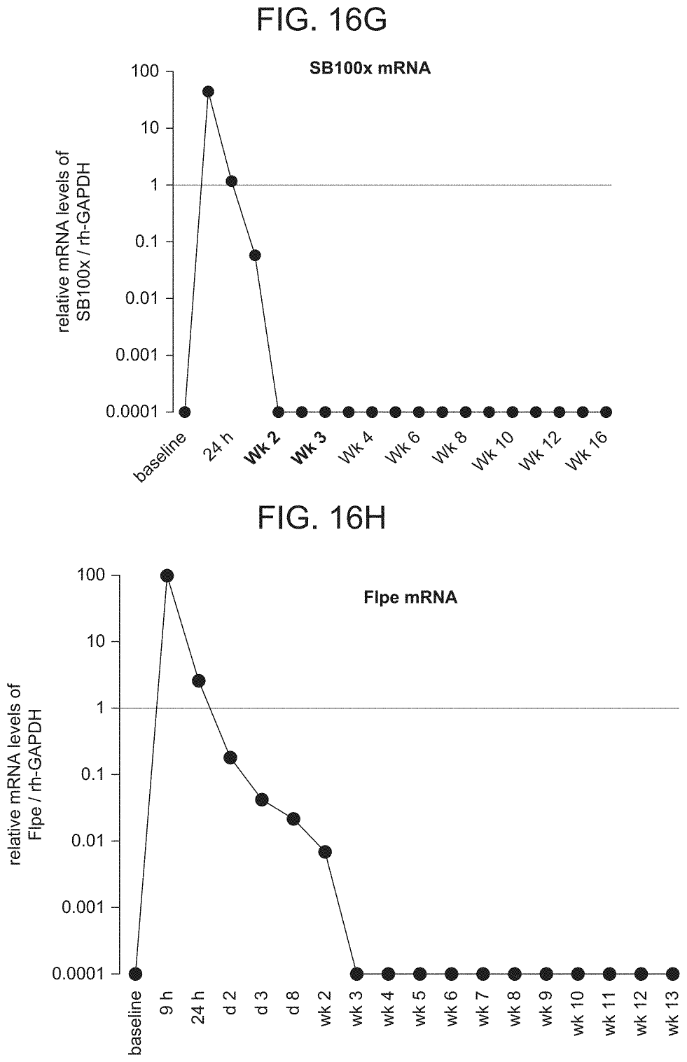

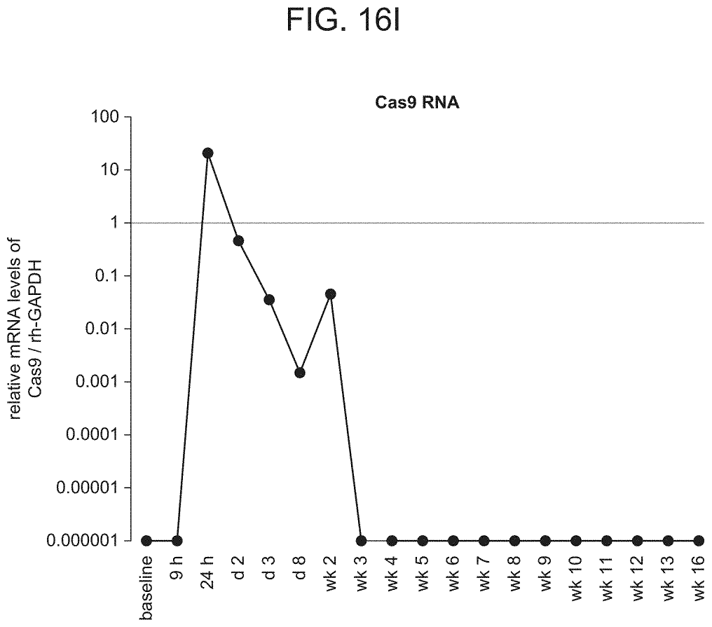

Expression of editing enzymes and antibody responses: Because of the episomal nature of the HDAd-SB vector and the Cas9 self-destruction mechanism (see FIG. 8A), expression of the genome editing enzymes (SB100x, Flpe, and Cas9) was lost by week 3 as shown by qRT-PCR for corresponding mRNA (FIGS. 16G-16I). Consequently, no significant IgM and IgG antibody responses were observed against encoded Flpe, SB100x, and Cas9 products, though Flpe, SB100x and Cas9 mRNA were detectable, but a response was detected against GFP (see, e.g., FIGS. 16C-16I for data from NHP #1). However, as expected, intravenous injection of HDAd5/35++ triggered IgM and IgG responses directed against the HDAd virus capsid proteins (see, e.g., NHP #1 data provided in FIG. 16A, NHP #3 data provided in FIG. 16B). These responses subsided over time. Notably, at the time of HDAd injection, NHP #1 had an anti-HDAd titer of 1:680, most likely due to exposure to Ad during the 4 weeks of quarantine.

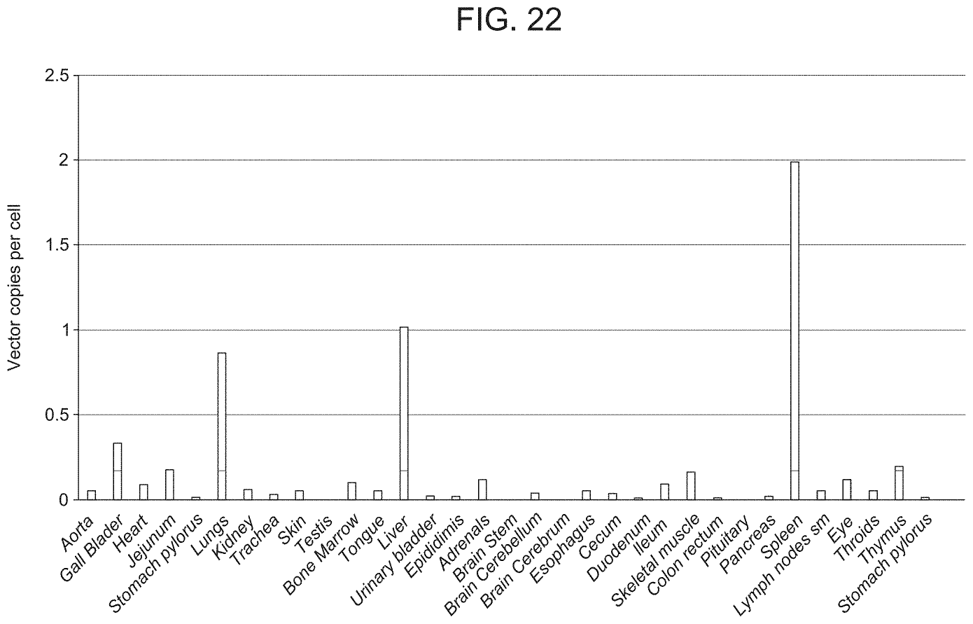

Vector biodistribution at day 3: Serum and tissue samples from animal #2 (euthanized at day 3 because of an accidental overdose of tacrolimus) showed that the new immune suppression regimen (dexamethasone, IL-6R, IL-1bR antagonists, saline bolus IV) mitigated all side effects associated with HDAd5/35++ vector administration. Vector DNA biodistribution studies demonstrated very low or absent transduction of most tissues (including testes and CNS) (FIG. 22). Vector signals in the lungs, liver, and spleen were derived from residual blood cells (even though the animal had been perfused with 5 liters of PBS before tissue collection.) Week 24 bone marrow samples were used for RNA-seq to assess potential side effects of gene addition/editing on the transcriptome (in comparison a bone marrow sample collected before the start of the study) (see excel file). No significant abnormalities were found.

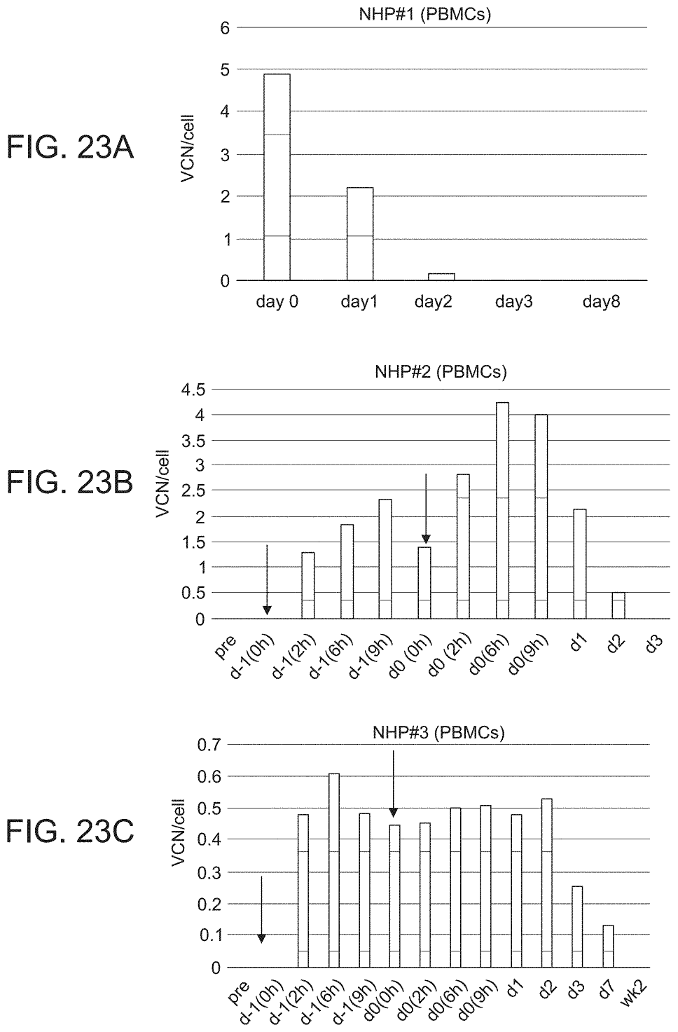

Transduction of PBMCs: The vector copy number (VCN) in PBMCs was measured at different time points after HDAd injection (FIGS. 23A-23C). The data indicate early transduction of PBMCs. However, vector signals were lost by week 2, most likely due to the natural turnover of differentiated blood cells and/or episomal vector DNA degradation.

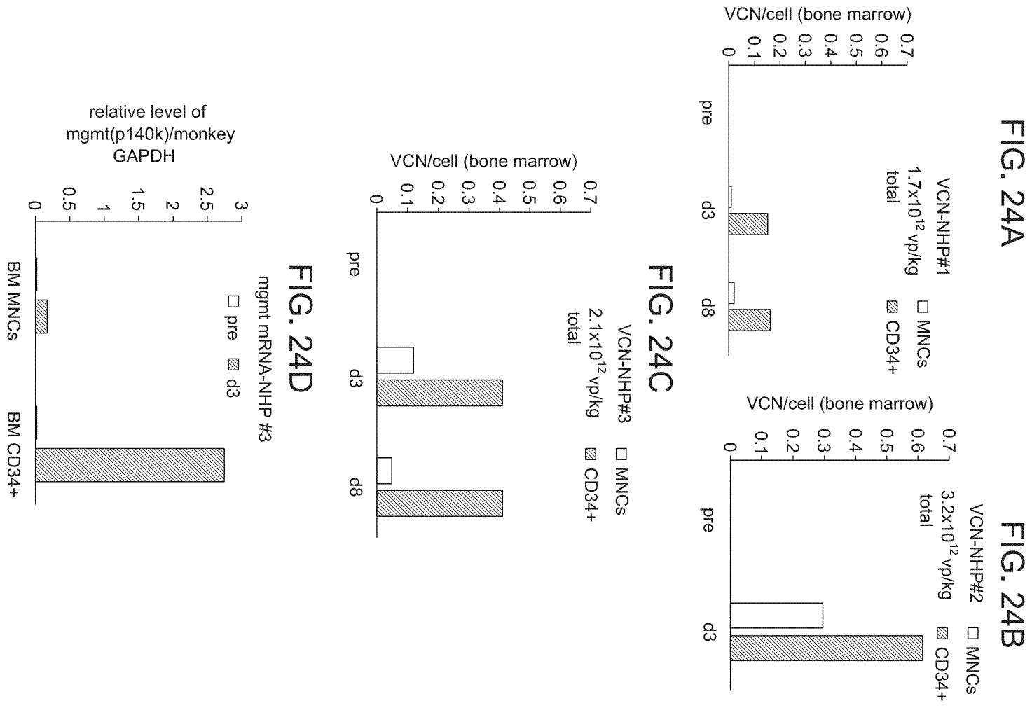

Preferential transduction of HSCs: Analysis of total bone marrow mononuclear cells (MNCs) and bone marrow CD34+ cells at days 3 and 8 after HDAd injection showed vector-positive cells. The vector copy number (VCN) per cell depended on the virus dose injected. Importantly, the data indicated preferential transduction of mobilized CD34+ cells that then returned to the bone marrow (FIGS. 24A-24D). The vector signals detected at these early time points most likely originate from episomal vector DNA.

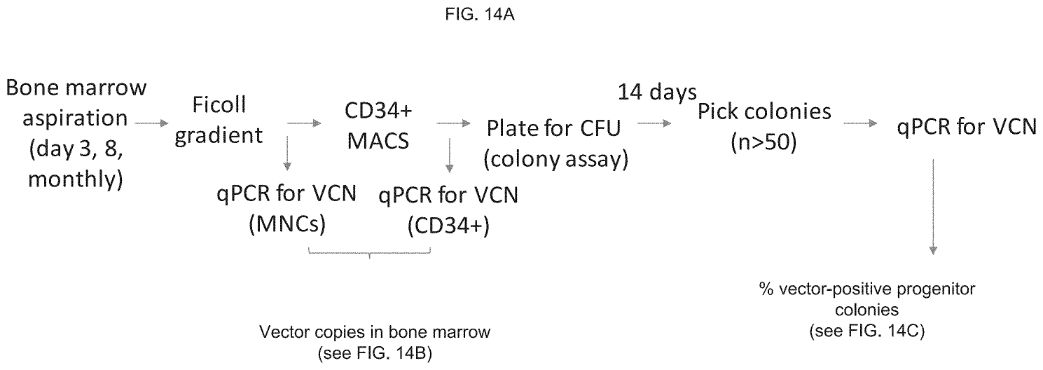

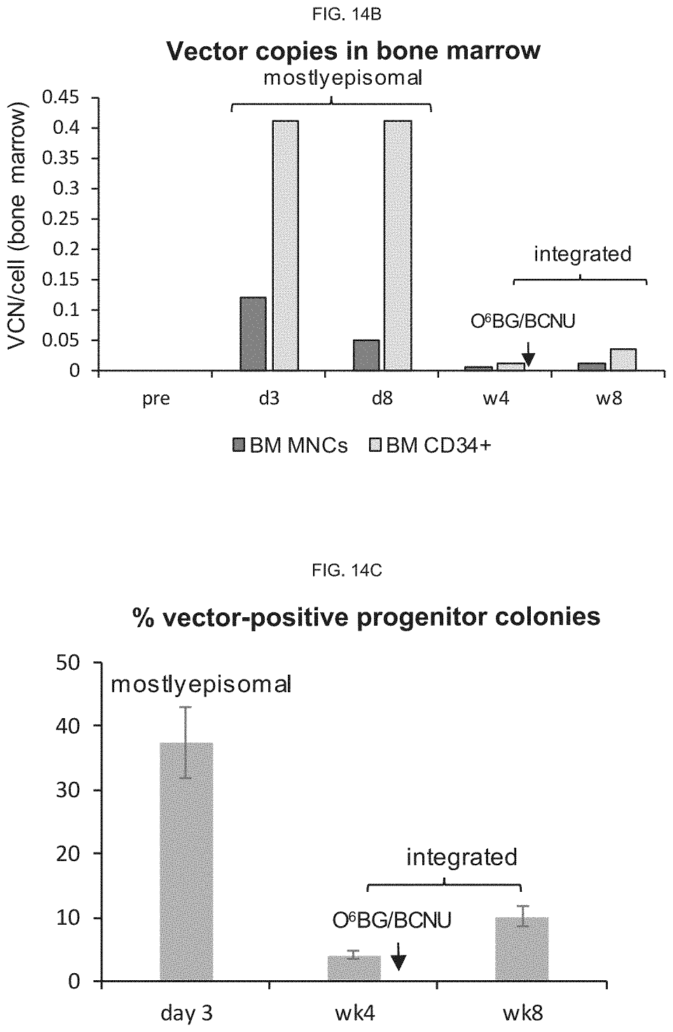

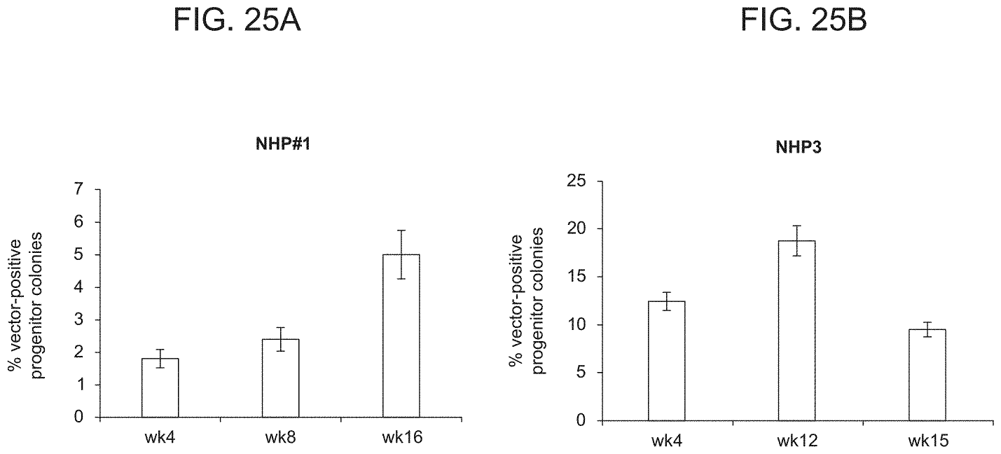

Stable HSC transduction: To measure transgene integration, bone marrow cells were plated for CFU assays. During colony formation/cell proliferation most of the episomal vector is lost (see, e.g., data from NHP #3 collected according to FIG. 14A and shown in FIGS. 14B and 14C). DNA analysis from single colonies with a VCN of 1 suggested an integration frequency of 5-10% (see FIGS. 25A and 25B). This frequency did not significantly increase by in vivo selection. The latter is thought to occur at the level of progenitors, not at the level of primitive HSCs/CFUs.

Data from NHP #1

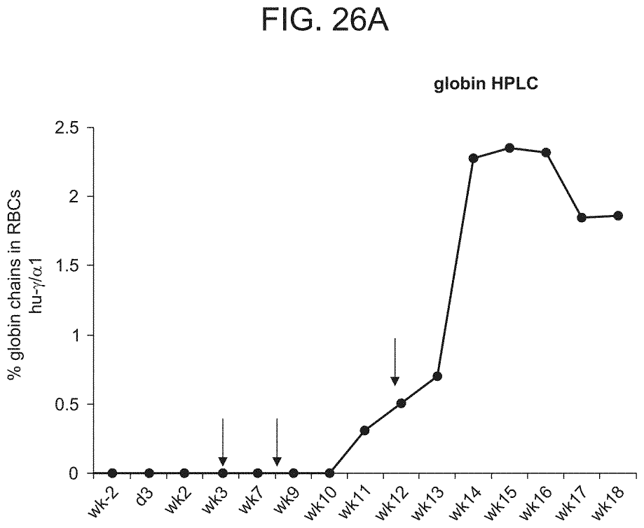

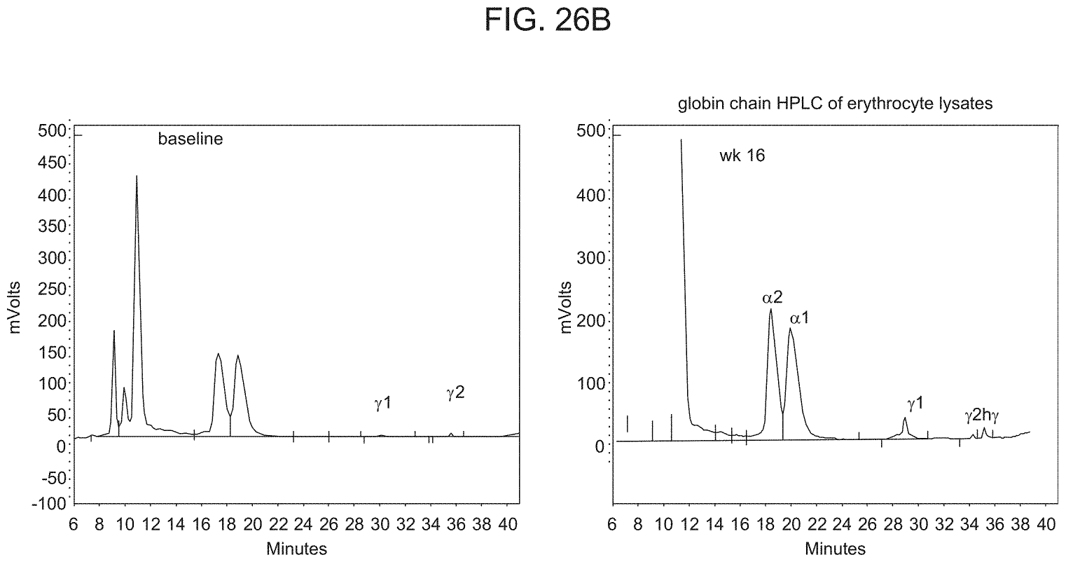

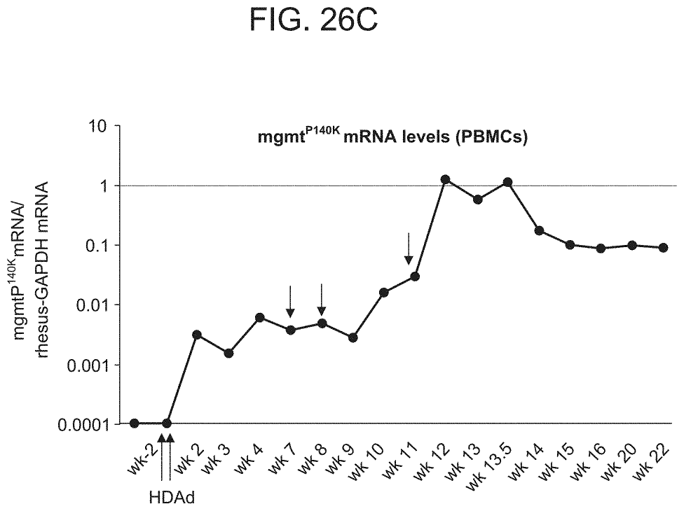

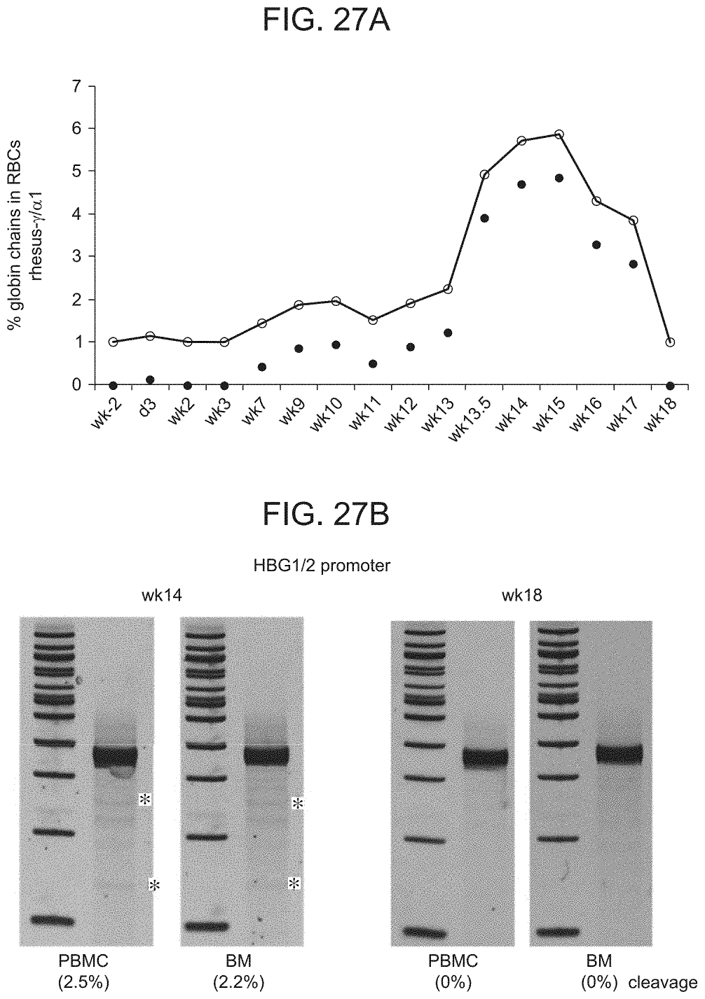

NHP #1 had pre-existing anti-HDAd5/35++ serum antibodies and received 0.5×10¹² vp/kg HDAd on day −1 and 1.2×10¹²vp/kg on day 0. Because of this, initial CD34+ cell transduction was less efficient than in the other two animals (see FIGS. 24A-24D, 25A, and 25B). Nevertheless, γ-globin became detectable by flow cytometry on 20% of peripheral red blood cells after week 2 (FIG. 15A-15C). In vivo selection with O⁶BG/BCNU temporarily decreased γ-globin marking, most likely due to cytotoxic effects on erythroid progenitors. However, after the third cycle of O⁶BG/BCNU treatment, the percentage of γ-globin-positive RBCs climbed to 90% and remained stable until the end of the experiment, except for a transient decrease around week 18. A similar pattern was seen in erythroid progenitors in the bone marrow. HPLC analysis of RBC lysates allowed us to distinguish endogenous rhesus γ-globin from added human γ-globin. γ-globin levels were expressed relative to al globin chains to which they bind to form HbF. Human γ-globin chains became detectable by HPLC after the completion of in vivo selection and remained relatively stable until week 22 at 1% of al globin (FIGS. 26A, 26B). In line with human γ-globin is the expression kinetics of the other transgene, human mgmt^{P140K}. In vivo selection increased the level of mgmt^{P140K} mRNA by two orders of magnitude, reaching levels comparable to GAPDH mRNA expression (2000 mRNA copies per cell). This is a robust level that can confer resistance to O⁶BG/BCNU treatment. Detectable mgmt^{P140K} mRNA decreased to 10% of GAPDH mRNA at week 15. After week 15 it remained stable (FIG. 26C). In vivo selection also resulted in an increase of rhesus γ-globin starting at week 16 and reaching a peak at week 15 with 6% of al globin (FIG. 27A). The subsequent decline could be due to the loss of CRISPR-edited cells with detrimental genomic rearrangements. This is in part supported by T7E1 mismatch PCR data, which showed 2.5% indels in the HBG1/2 promoter target site at week 14 but undetectable cleavage at week 21 (FIG. 27B). Expression of human and rhesus mRNA mirrored that of the corresponding protein levels. Overall, the human γ-globin and mgmt^{P140K} expression data indicate vector integration at levels that allowed for in vivo selection/expansion of transduced progenitors/peripheral blood cells. With the decrease in rhesus γ-globin expression after week 15, the high γ-globin marking detected on RBCs after week 15 by flow cytometry (FIG. 25A), is, most likely, based on the expression of the human γ-globin form. No abnormalities in genome and transcriptome analyses of NHP #1 were found at the time of scheduled necropsy.

Efficacy Data from NHP #3

This animal had a pre-injection anti-HDAd antibody titer of 1:66, ten-fold lower than NHP #1. The virus dose injected with 2.1×10¹² vp/kg, 20% higher than NHP #1. NHP #3 received SC tacrolimus until week 15. Immunosuppression was resumed at week 18 with tacrolimus/MM F/abatacept.

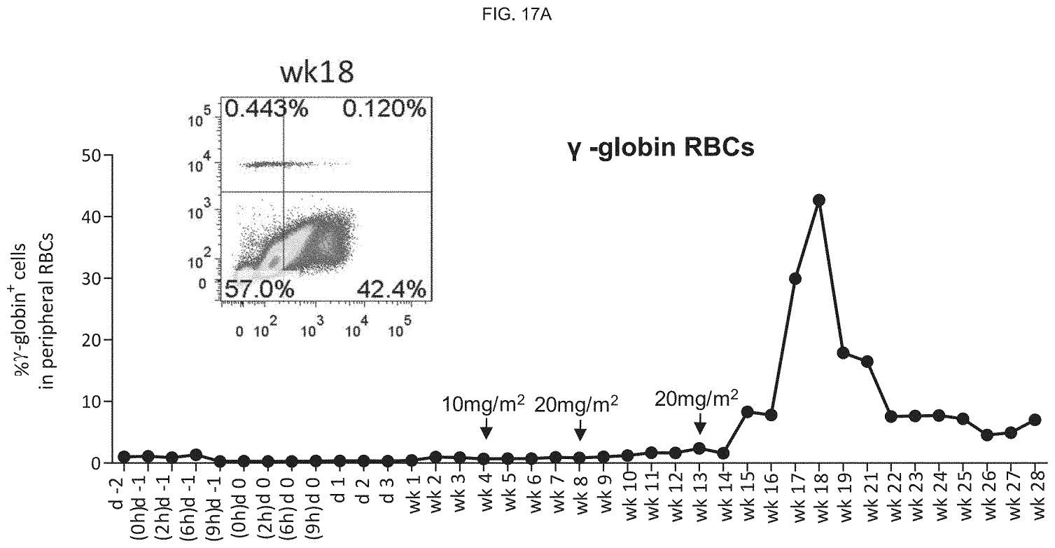

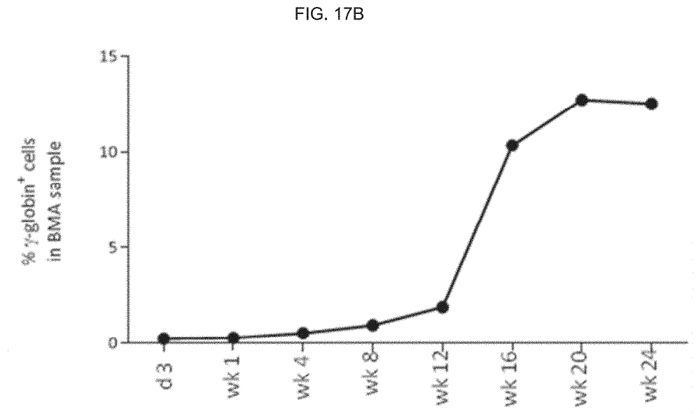

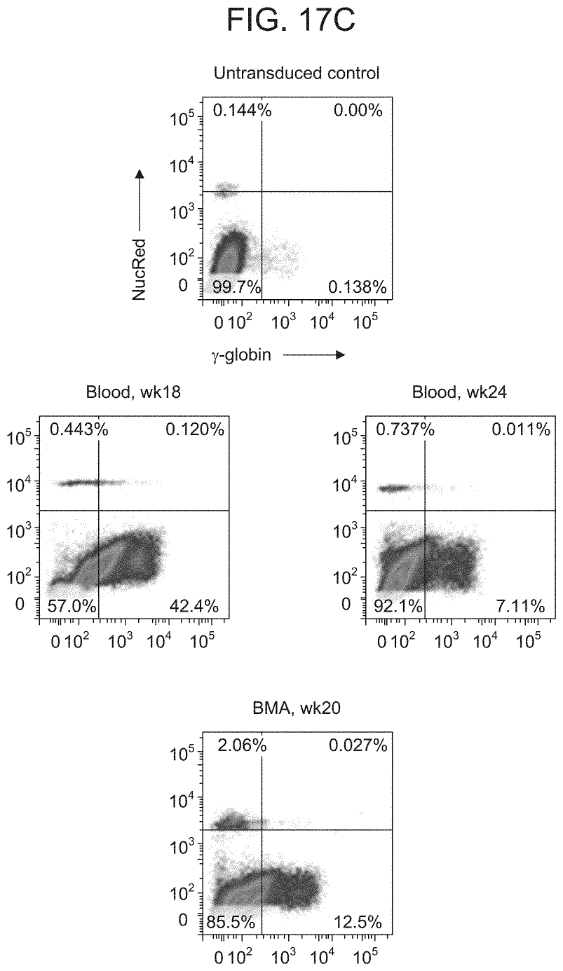

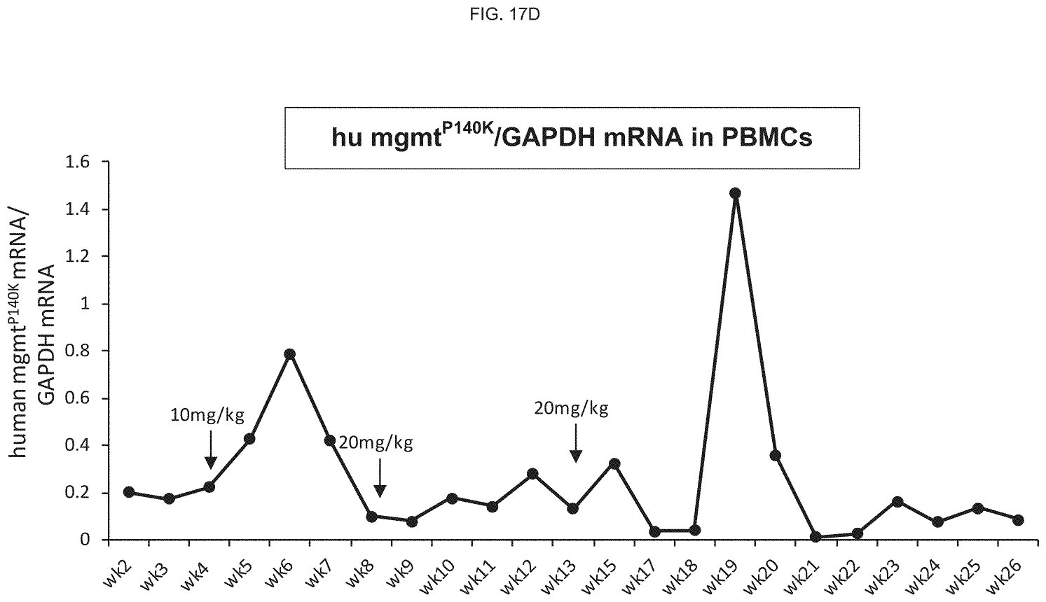

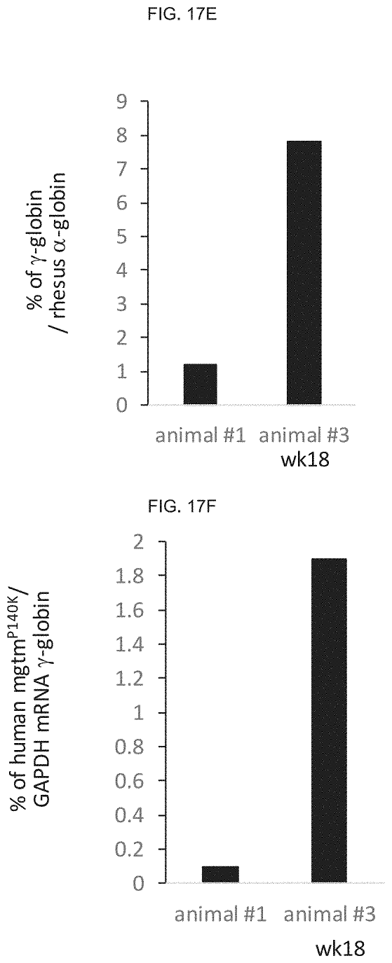

Initial CD34+ transduction (d3 and 8) and the percentage of stably transduced CFU was at least two-fold higher than in NHP #1 (FIGS. 24A-24D, 25A, and 25B). The vector used in NHP #3 contained the rhesus γ-globin gene to reduce immune responses against the therapeutic protein. Unexpectedly, rhesus γ-globin was less efficiently expressed than the human γ-globin gene when it was combined with a human-β-globin LCR. γ-globin marking of RBCs and expression levels therefore underestimate the potential of the in vivo approach. The percentage of γ-globin-positive RBCs started to increase after the third cycle of in vivo selection reaching 45% at week 18, however marking decreased to 7% by week 21, remaining relatively stable until the end of the study (week 28) (FIG. 17A). This decline was less pronounced in bone marrow mononuclear cells (FIGS. 17B, 17C). Similar to NHP #1, the vast majority of γ-globin measured by flow cytometry originated from the added rhesus γ-globin gene. CRISPR-edited cells appeared to be lost (FIG. 27B).

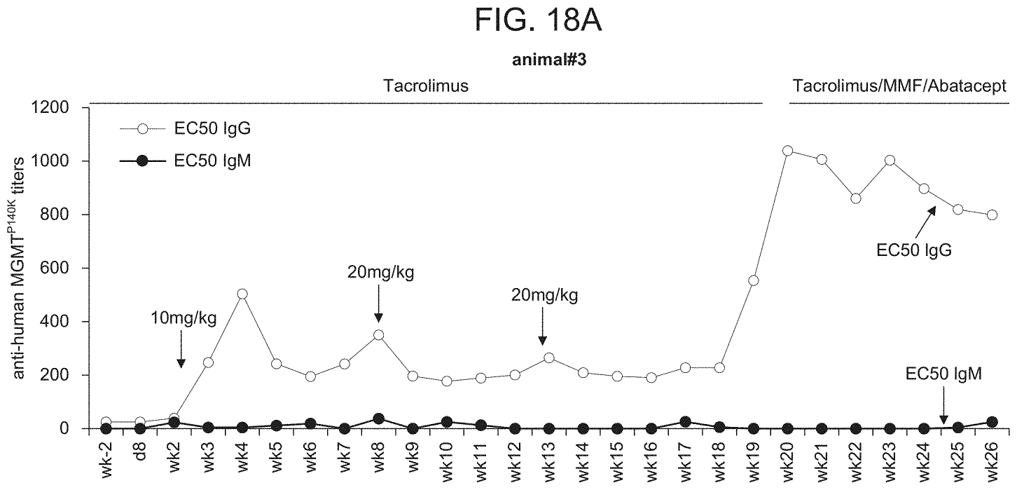

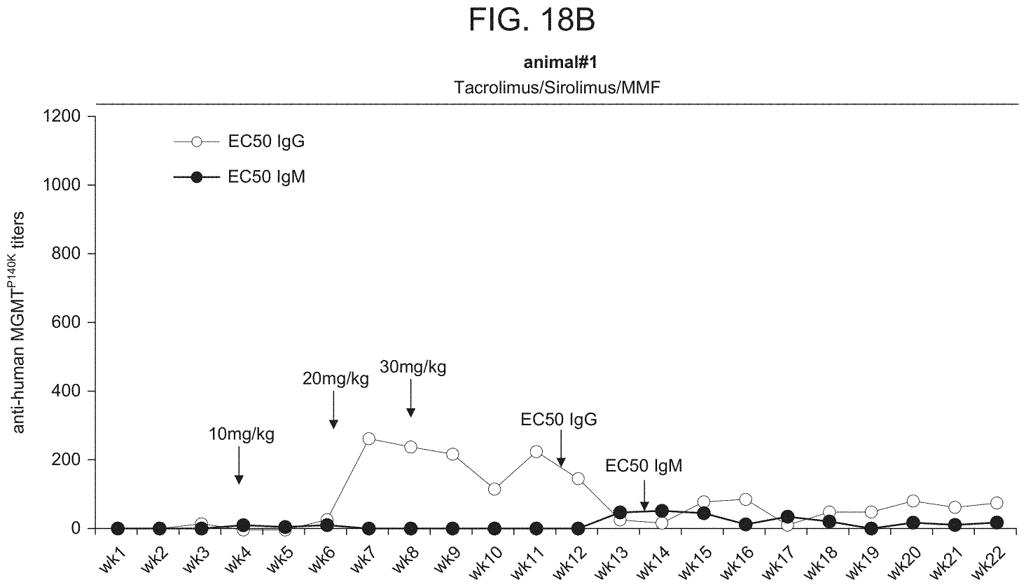

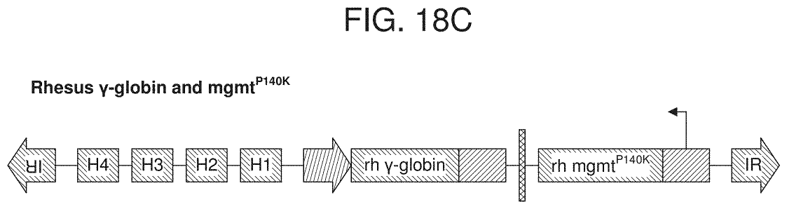

The level of human mgmt^{P140K} mRNA expression in PBMCs also increased after the first round of in vivo selection, however, increases after the 2nd and 3rd cycle were blunted. mgmt^{P140K} expression was shortly restored after restarting immunosuppression with tacrolimus+MMF+Abatacept (FIG. 17D). This indicates a role of anti-human MGMT^{P140K} immune responses, specifically T-cell responses capable of eliminating transgene expressing cells in the periphery, and, to a lesser degree, in the bone marrow. Serum IgG antibodies against human MGMT support this hypothesis (FIGS. 18A and 18B). Overall, however, higher transgene expression levels were observed in NHP #3 than in NHP #1 (FIGS. 17E and 17F.). Taking the mgmt^{P140K} mRNA and antibody data together, one can speculate that whenever mgmt^{P140K} levels increase (e.g. after O⁶BG/BCNU), there is more immune stimulation which cannot be completely controlled by tacrolimus. When tacrolimus was discontinued for 2 weeks (weeks 15 to 17), anti-MGMT antibody levels increased, and additional treatment with MMF and CTLA4-Ig (abatacept) only partially counteracted the immune response. Notably, triple immunosuppressant treatment stopped the further decline in γ-globin and mgmt^{P140K} expression (weeks 22-28). FIGS. 18A and 18B also show that the more aggressive immunosuppression regimen in NHP #1 (tacrolimus/sirolimus/MMF) can mitigate anti-MGMT immune responses.

Future NHP embodiments of this work could include one or more of i) replacement of human mgmt^{P140K} gene with the rhesus version of this mutant (see, for example, FIG. 18C), and ii) administering Abatacept at days −1, 7, 14 and then monthly to trigger some level of tolerance to foreign transgene products. Importantly, immune responses against human transgene products should not be a problem in humans.

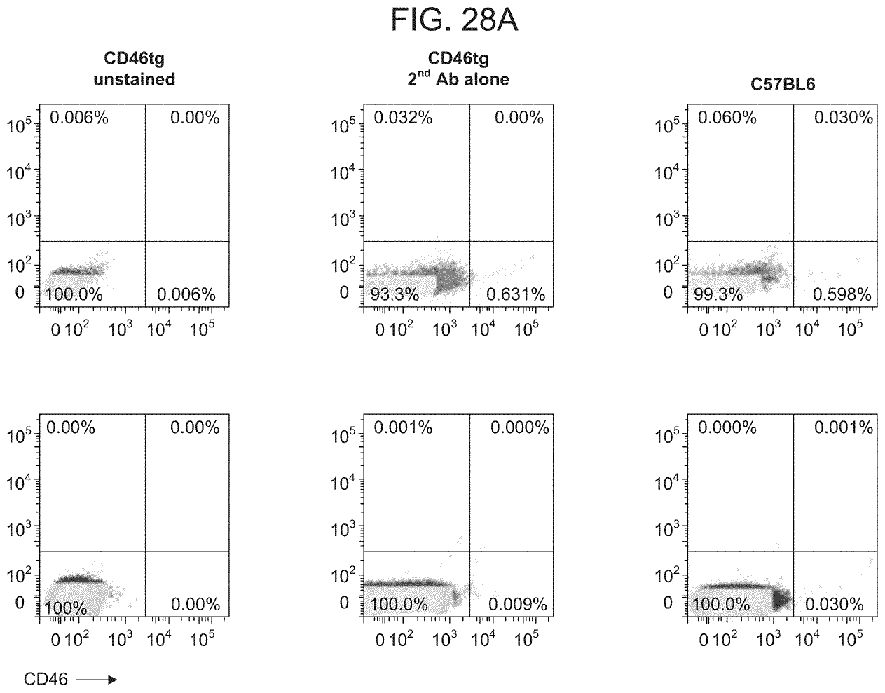

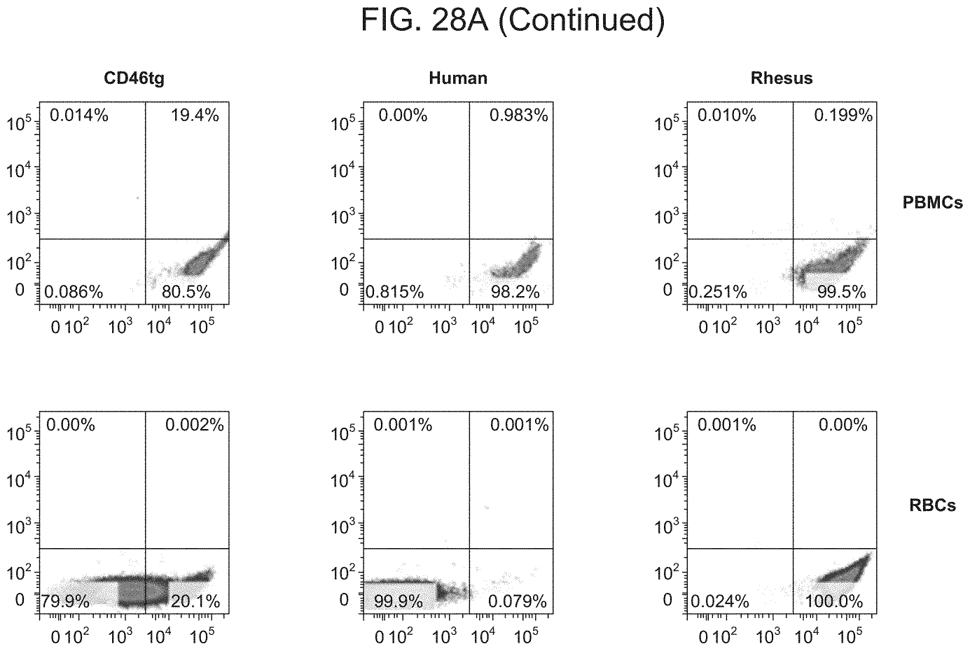

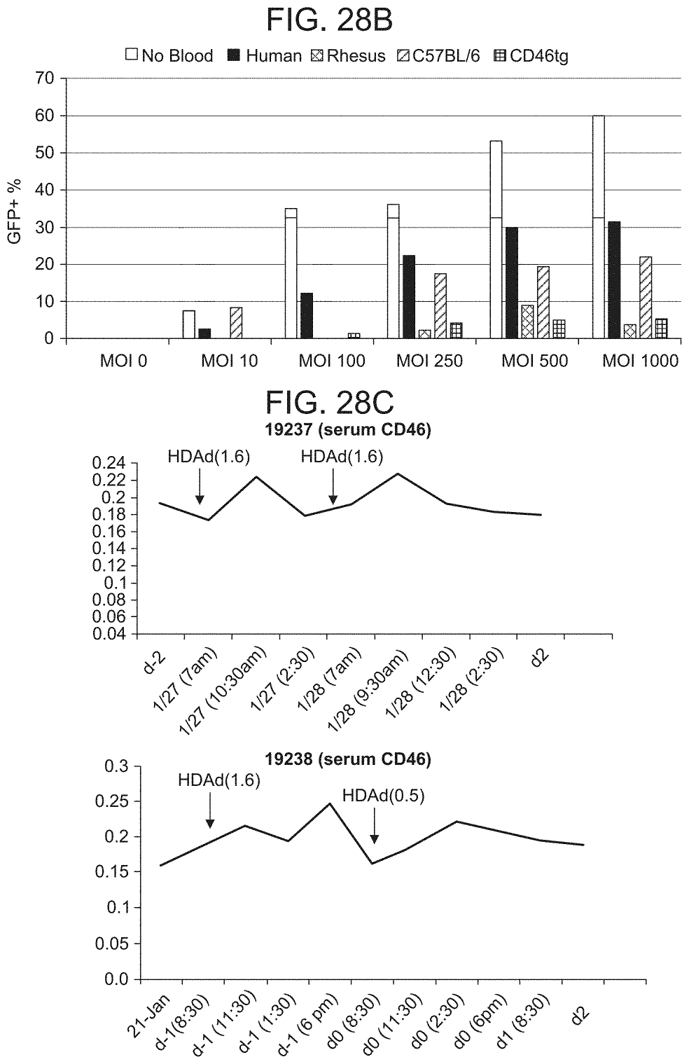

CD46 on rhesus erythrocytes. Unlike in human, rhesus erythrocytes possess CD46 on their surface (FIG. 28A). Binding of ligands, including HAd5/35++, to CD46 results in shedding of the extracellular domain of CD46 (Sakurai et al., Gene Ther. 14(11): 912-919, 2007). Considering the enormous number of erythrocytes in blood, erythrocytes in NHPs create a major sink for unspecific sequestration/loss of IV injected HDAd5/35++ vector particles. It also makes “vector dose—response” studies difficult. Data supporting that rhesus erythrocytes can block HDAd5/35++ injection in vitro are shown in FIG. 28B. On the other hand, recent data by Zafar et al. (Cancer Gene Therapy, 2020, doi.org/10.1038/s4147-020-00226-z) indicate that Ad binding to erythrocytes is reversible. The slow (bi-phasic) serum clearance of HDAd genomes (FIGS. 19A-19C) and the changes in sCD46 concentrations in serum after HDAd injection (FIG. 28C) could indicate that this observation is correct. Nevertheless, to saturate CD46 on erythrocytes, high vector doses were injected in two cycles. Because acute responses can be controlled, this line will be followed.

In summary, this Example demonstrates, among other things, that an IL-1 signal inhibitor (e.g., anakinra) is a potent agent for suppressing in vivo immune responses to adenoviral vector administration, can be used in combination with other agents such as an IL-6 signal inhibitor (e.g., tocilizumab) and/or a corticosteroid (e.g., dexamethasone), and can fully blunt the innate immune response to an exemplary adenoviral vector (HDAd5/35++). This documents a role for IL-1 and/or IL-6 in driving the innate response to these vectors. This Example further demonstrates that: in vivo HSC transduction with HDAd5/35++ can be safely done in mobilized NHPs with adequate immune suppression; 5% of CFU are stably transduced (before in vivo selection); transgene marking/expression in peripheral blood cells can be increased by in vivo selection; with a fully intact immune system, and CRISPR/Cs9-edited cells may be lost over time.

### Example 6: In Vivo HSC Gene Therapy for Hemoglobinopathies: In Vivo Gene Therapy in Rhesus Macaques Including a Viral Vector, a Support Vector, an Immune Suppression Regimen, a Selecting Agent, and a Stem Cell Mobilization Regimen

The present Example further illustrates use of an immunosuppression regimen including tocilizumab, anakinra, and dexamethasone. Reagents and experimental design were as in Example 5 except as otherwise noted here. Tocilizumab is an exemplary IL-6 receptor antagonist, anakinra is an exemplary IL-1 receptor antagonist, and dexamethasone is an exemplary corticosteroid. The present disclosure includes the recognition that combination of an IL-6 receptor antagonist (e.g., tocilizumab) and an IL-1 receptor antagonist (e.g., anakinra), optionally further in combination with a corticosteroid (e.g., dexamethasone), is an unexpectedly potent combination for suppression of the increase in cytokine levels (in particular IL-6 and/or TNF) that otherwise results from administration of a viral vector (e.g., an adenoviral vector) to a mammal, e.g., for purposes of in vivo gene therapy. Non-human primates of the present disclosure were administered tocilizumab, anakinra, and dexamethasone. To the extent additional agents were administered to NHPs, those of skill in the art will appreciate that such additional agents are not necessary for the beneficial suppression of cytokine levels in connection with administration of a viral vector to a mammal. For example, tacrolimus is administered to suppress adaptive immune responses.

A major risk with systemic administration of viral vectors to a mammalian subject (e.g., administration of an adenoviral vector) is the activation of the innate immune system. An acute innate immune response occurs soon after vector delivery (i.e., minutes to hours) and is dose-dependent. For example, elevation of serum IL-6 can rise at 1 hr following systemic administration of adenoviral vector and reach a peak level between 3 to 6 hrs after administration. This innate immune response can constitute or result in acute toxicity; the increase in cytokine levels can include a cytokine storm, which can result in death.

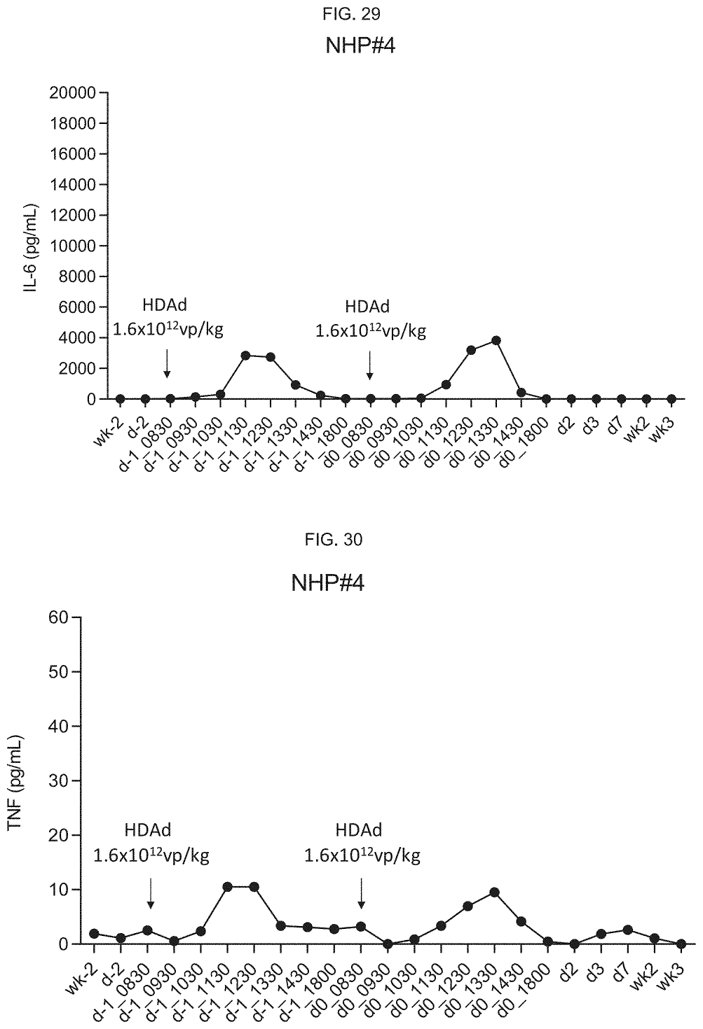

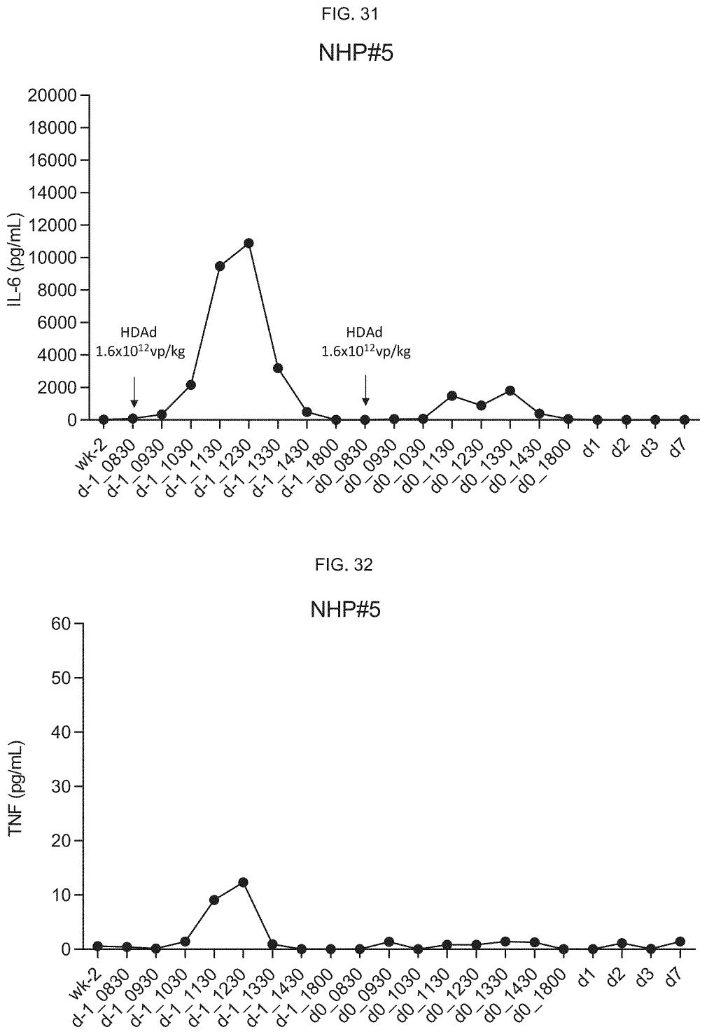

The present Examples include the finding that anakinra, tocilizumab, and dexamethasone attenuate innate immune activation, substantially reducing cytokine release brought upon by intravenous administration of Ad5/35++. NHP #4 and NHP #5 were administered an immune suppression regimen including tocilizumab, anakinra, and dexamethasone (see Table 6). Anakinra was administered half an hour prior to HDAd dosing. Time to peak serum anakinra concentration is between 2.5 to 4.5 hours following subcutaneous administration, as estimated from available information. IL-6 levels measured in NHP #4 and NHP #5 confirm the suppression of IL-6 levels by the administered agents (FIGS. 29 and 31). TNF levels measured in NHP #4 and NHP #5 confirm the suppression of TNF levels by the administered agents (FIGS. 30 and 32). Thus, the present Example confirms that administration of anakinra, tocilizumab, and dexamethasone is effective for suppression of innate immune response as measured by IL-6 and/or TNF levels.

TABLE 6
Immune suppression regimen

Immune Suppression Agents Administered, and Doses

| Day | NHP#4 | NHP#5 |
|---|---|---|
| −6 | | |
| −5 | | |
| −4 | | |
| −3 | | |
| −2 | dexamethasone | dexamethasone |
| | (4.0 mg/kg iv) qd | (4.0 mg/kg iv) qd |
| −1* | dexamethasone | dexamethasone |
| | (4.0 mg/kg) bid | (4.0 mg/kg) bid |
| | tocilizumab | tocilizumab |
| | (8.0 mg/kg iv) bid | (8.0 mg/kg iv) bid |
| | anakinra | anakinra |
| | (50 mg/animal sc) bid | (50 mg/animal sc) bid |
| 0* | dexamethasone | dexamethasone |
| | (4.0 mg/kg iv) bid | (4.0 mg/kg iv) bid |
| | tocilizumab | tocilizumab |
| | (8.0 mg/kg iv) bid | (8.0 mg/kg iv) bid |
| | anakinra | anakinra |
| | (50 mg/animal sc) bid | (50 mg/animal sc) bid |
| 1 | anakinra | |
| | (50 mg/animal sc) qd | |
| 2 | anakinra | anakinra |
| | (50 ma/animal sc) ad | (50 ma/animal sc) ad |

*Days on which adenoviral vector was administered

Other Embodiments. While a number of embodiments have been described, it is apparent that the basic disclosure and examples may provide other embodiments that utilize or are encompassed by the compositions and methods described herein. Therefore, it will be appreciated that the scope is to be defined by that which may be understood from the disclosure and the appended claims rather than by the specific embodiments that have been represented by way of example. All references cited herein are hereby incorporated by reference.

### SUMMARY OF SEQUENCE(S)

The nucleic acid and/or amino acid sequences described herein are shown using standard letter abbreviations, as defined in 37 C.F.R. § 1.822. A computer readable text file, entitled “F053-0131 US_SeqList.txt” created on or about Oct. 5, 2022, with a file size of 4 KB, contains the sequence listing for this application and is hereby incorporated by reference in its entirety.

In the accompanying Sequence Listing, SEQ ID NO: 1 is the amino acid sequence of anakinra, as follows:

MRPSGRKSSKMQAFRIWDVNQKTFYLRNNQLVAGYLQGPNVNLEEKIDV
VPIEPHALFLGIHGGKMCLSCVKSGDETRLQLEAVNITDLSENRKQDKR
FAFIRSDSGPTTSFESAACPGWFLCTAMEADQPVSLTNMPDEGVMVTKF
YFQEDE

What is claimed is:

1. A method of in vivo gene therapy in a mammalian subject, the method comprising:
(i) administering to the subject an immune suppression regimen comprising an inflammatory signal inhibitor; and
(ii) administering to the subject at least one dose of a viral gene therapy vector.

2. A method of transducing stem cells of a mammalian subject without removal of the stem cells from the subject, the method comprising delivering a viral gene therapy vector to a subject having been administered an immune suppression regimen comprising an inflammatory signal inhibitor.

3. The method of claim 1 or 2, wherein the inflammatory signal inhibitor is an interleukin-1 (IL-1) signal inhibitor, optionally wherein the IL-1 signal inhibitor is an IL-1 receptor (IL-1R) antagonist.

4. The method of claim 3, wherein the IL-1R antagonist is anakinra.

5. The method of any one of claims 1-4, wherein the immune suppression regimen further comprises an interleukin 6 (IL-6) receptor antagonist.

6. The method of claim 5, wherein the IL-6 receptor antagonist is tocilizumab.

7. The method of any one of claims 1-6, wherein the immune suppression regimen further comprises a corticosteroid.

8. The method of claim 7, wherein the corticosteroid is dexamethasone.

9. The method of any one of claims 1-8, wherein the immune suppression regimen further comprises a calcineurin inhibitor.

10. The method of claim 9, wherein the calcineurin inhibitor is tacrolimus.

11. The method of any one of claims 1-10, wherein the immune suppression regimen further comprises a TNF-α signal inhibitor.

12. The method of claim 11, wherein the TNF-α signal inhibitor is selected from the group consisting of etanercept, infliximab, adalimumab, certolizumab, pegol, and golimumab.

13. The method of any one of claims 1-12, wherein the immune suppression regimen further comprises a JAK signal inhibitor.

14. The method of claim 13, wherein the JAK signal inhibitor is selected from the group consisting of baricitinib, tofacitinib, ruxolitinib, and filgotinib.

15. The method of any one of claims 1-12, wherein the administering of the immune suppression regimen comprises administering an IL-1 receptor antagonist to the subject:
(i) on the day prior to administration of a first dose of the vector;
(ii) on the day of administration of a first dose of the vector, optionally including at least one dose of IL-1 receptor antagonist 1 to 3 hours prior to administration of the first dose of the vector;
(iii) on the day of administration of one or more subsequent doses of the vector, optionally including at least one dose of IL-1 receptor antagonist 1 to 3 hours prior to administration of the one or more subsequent doses of the vector;
(iv) on each day between the day of administration of a first dose of the vector and the day of administration of a last dose of the vector; and/or
(v) on each of one, two, or more days after the day of administration of a last dose of the vector;
optionally wherein the IL-1 receptor antagonist is anakinra.

16. The method of any one of claims 1-15, wherein the administering of the immune suppression regimen comprises administering to the subject a single dose of IL-1 receptor antagonist per day or a plurality of doses of IL-1 receptor antagonist per day, optionally wherein the IL-1 receptor antagonist is anakinra.

17. The method of claim 15, wherein the administering of the immune suppression regimen comprises administering to the subject 0.01 to 20 mg/kg/day anakinra, optionally wherein the administration is intravenous or subcutaneous.

18. The method of claim 15, wherein the administering of the immune suppression regimen comprises administering to the subject 10 to 200 mg/day anakinra, optionally wherein the administration is intravenous or subcutaneous.

19. The method of any one of claims 1-18, wherein the administering of the immune suppression regimen comprises administering an IL-6 receptor antagonist to the subject:
(i) on the day prior to administration of a first dose of the vector;
(ii) on the day of administration of a first dose of the vector, optionally including at least one dose of IL-6 receptor antagonist no more than 1 hour prior to administration of the first dose of the vector;
(iii) on the day of administration of one or more subsequent doses of the vector, optionally including at least one dose of IL-6 receptor antagonist no more than 1 hour prior to administration of the one or more subsequent doses of the vector;
(iv) on each day between the day of administration of a first dose of the vector and the day of administration of a last dose of the vector; and/or
(v) on each of one, two, or more days after the day of administration of a last dose of the vector;
optionally wherein the IL-6 receptor antagonist is tocilizumab.

20. The method of any one of claims 1-19, wherein the administering of the immune suppression regimen comprises administering to the subject a single dose of IL-6 receptor antagonist per day or a plurality of doses of IL-6 receptor antagonist per day, optionally wherein the IL-6 receptor antagonist is tocilizumab.

21. The method of claim 19 or 20, wherein the administering of the immune suppression regimen comprises administering to the subject 1-15 mg/kg/day tocilizumab or 5-200 mg/day tocilizumab, optionally wherein the administration is intravenous.

22. The method of any one of claims 1-21, wherein the administering of the immune suppression regimen comprises administering a corticosteroid to the subject:
(i) on the day prior to administration of a first dose of the vector;
(ii) on the day of administration of a first dose of the vector;
(iii) on the day of administration of one or more subsequent doses of the vector;
(iv) on each day between the day of administration of a first dose of the vector and the day of administration of a last dose of the vector; and/or
(v) on each of one, two, or more days after the day of administration of a last dose of the vector;
optionally wherein the corticosteroid is dexamethasone, prednisone, prednisolone, methylprednisolone, triamcinolone, paramethasone, or betamethasone.

23. The method of any one of claims 11-22, wherein the administering of the immune suppression regimen comprises administering to the subject a single dose of corticosteroid per day or a plurality of doses of corticosteroid per day, optionally wherein the corticosteroid is dexamethasone.

24. The method of claim 22 or 23, wherein the administering of the immune suppression regimen comprises administering to the subject 0.1-10 mg/kg/day dexamethasone, optionally wherein the administration is intravenous, oral, or intramuscular.

25. The method of any one of claims 1-24, wherein the administering of the immune suppression regimen comprises administering a calcineurin inhibitor to the subject:
(i) on each of the four days prior to administration of a first dose of the vector;
(ii) on the day of administration of a first dose of the vector;
(iii) on the day of administration of one or more subsequent doses of the vector; and/or
(iv) on each day between the day of administration of a first dose of the vector and the day of administration of a last dose of the vector; and/or
(v) on each of one, two, or more days after the day of administration of a last dose of the vector;
optionally wherein the calcineurin inhibitor is tacrolimus.

26. The method of any one of claims 1-25, wherein the administering of the immune suppression regimen comprises administering to the subject a single dose of calcineurin inhibitor per day or a plurality of doses of calcineurin inhibitor per day, optionally wherein the calcineurin inhibitor is tacrolimus.

27. The method of claim 25 of 26, wherein the administering of the immune suppression regimen comprises administering to the subject 0.001-0.1 mg/kg/day tacrolimus, optionally wherein the administration is subcutaneous.

28. The method of any one of claims 1-27, wherein the method (i) does not cause a significant increase in the amount of one or more of IFN-g, TNF, IL-2, IL-4, IL-5, or IL-6; or (ii) causes a significantly smaller increase in the amount of one or more of IFN-g, TNF, IL-2, IL-4, IL-5, or IL-6 as compared to a control that does not comprise one or more immune suppression agents, optionally wherein the control does not comprise one or more immune suppression agents selected from (a) the inflammatory signal inhibitor; (b) the IL-6 receptor antagonist; (c) the corticosteroid; and (d) the calcineurin inhibitor; optionally wherein the amount is measured by ELISA or a cytokine bead array.

29. The method of any one of claims 1-28, wherein the method further comprises administering to the subject a stem cell mobilization regimen.

30. The method of any one of claims 1-29, wherein the vector comprises a nucleic acid sequence that encodes a selectable marker, optionally wherein the selectable marker is MGMT^{P140K}.

31. The method of claim 30, wherein the method comprises administering to the subject a selecting agent, optionally wherein the selectable marker is MGMT^{P140K} and the selecting agent is O⁶BG/BCNU.

32. The method of claim 30 or 31, wherein the selecting agent is administered to the subject in one or more doses, optionally wherein a first dose of the selecting agent is administered to the subject about 1 week, 2 weeks, 3 weeks, 4 weeks, 5 weeks, 6 weeks, 7 weeks, 8 weeks, 9 weeks, and/or 10 weeks after administration of a first dose of the vector to the subject.

33. The method of any one of claims 1-32, wherein the vector is administered to the subject by injection, optionally wherein the injection is intravenous or subcutaneous.

34. The method of any one of claims 1-33, wherein at least a first dose of the vector comprises at least 1E10, 1E11, or 1E12 viral particles per kilogram (vp/kg).

35. The method of any one of claims 1-34, wherein the vector is administered at a total dosage of at least 1E10, 1E11, 1E12, 2E12, or 3E12 vp/kg.

36. The method of any one of claims 1-35, wherein the vector is an adenoviral vector, adeno-associated viral vector, herpes simplex viral vector, retroviral vector, lentiviral vector, alphaviral vector, flaviviral vector, rhabdoviral vector, measles viral vector, Newcastle disease viral vector, poxviral vector, or picornaviral vector.

37. The method of any one of claims 1-36, wherein the vector is an adenoviral vector.

38. The method of any one of claims 1-37, wherein the vector is a group B adenoviral vector.

39. The method of any one of claims 1-38, wherein the vector is, or is derived from, an Ad5/35 or Ad35 adenoviral vector, optionally wherein the vector is an Ad35⁺⁺ or Ad5/35⁺⁺ adenoviral vector.

40. The method of any one of claims 1-39, wherein the vector is a replication incompetent vector, optionally wherein the replication incompetent vector is a helper-dependent adenoviral vector.

41. The method of any one of claims 1-40, wherein viral gene therapy vector comprises a nucleic acid comprising a therapeutic payload, and wherein the method further comprises administering to the subject a support vector encoding an agent that facilitates integration of the therapeutic payload into a target cell genome.

42. The method of claim 41, wherein the support vector is administered to the subject together with the viral gene therapy vector.

43. The method of claim 41 or 42, wherein the support vector is administered at a total dosage of 1E9 to 1E14 viral particles per kilogram (vp/kg).

44. The method of any one of claims 1-43, wherein the viral gene therapy vector comprises a nucleic acid comprising a therapeutic payload, and wherein the method causes delivery of the therapeutic payload to stem cells, optionally wherein delivery of the therapeutic payload comprises integration of the therapeutic payload into the genomes of the stem cells.

45. The method of any one of claims 1-44, wherein the viral gene therapy vector comprises a nucleic acid comprising a protein-encoding therapeutic payload, and, after administration of the vector to the subject, at least about 70%, about 80%, or about 90% of PBMCs of the subject express the protein.

46. The method of any one of claims 1-45, wherein the subject is a human subject.

47. The method of claim 46, wherein the human subject suffers from sickle cell anemia, thalassemia, thalassemia intermedia, hemophilia A, hemophilia B, von Willebrand Disease, Factor V Deficiency, Factor VII Deficiency, Factor X Deficiency, Factor XI Deficiency, Factor XII Deficiency, Factor XIII Deficiency, Bernard-Soulier Syndrome, Gray Platelet Syndrome.

48. The method of any one of claims 1-47, wherein the dosing regimen of one or more immune suppression agents of the immune suppression regimen is increased in unit dose, daily dose, total dose, frequency of doses, and/or total number of doses based on the measured level of an immunotoxicity biomarker in the subject or a sample from the subject after administration of at least one dose of the viral gene therapy vector, where the dosing regimen of the one or more immune suppression agents, is increased if the measured level is indicative of immunotoxicity.

49. The method of claim 48, wherein the immunotoxicity biomarker is selected from the group consisting of IL-Iβ, IL-2, IL-3, IL-4, IL-5, IL-6, IL-8, IL-10, IL-12, IL-13, IL-15, IL-17, IL-23, IL-27, IL-30, IL-36 IL-1Ra, IL-2R, IFN-α, IFN-b, IFN-γ, MIP-Ia, MIP-Iβ, MCP-1, TNF-α, TNF-β GM-CSF, G-CSF, CXCL9, CXCL10, VEGF, RANTES, EGF, HGF, FGF-8, CD40, CD40L, C-reactive protein, procalcitonin, ferritin, D-dimer, total population of lymphocytes, subpopulations of lymphocytes, subject temperature, and a combination thereof.

50. The method of any one of claims 1-49, wherein the dosing regimen of one or more immune suppression agents of the immune suppression regimen is increased in unit dose, daily dose, total dose, frequency of doses, and/or total number of doses based on the measured level of antibodies to the viral gene therapy vector in the subject or a sample from the subject after administration of at least one dose of the viral gene therapy vector, where the dosing regimen of the one or more immune suppression agents, is increased if the measured level is indicative of immunotoxicity, optionally wherein the measured level is an antibody titer, and optionally wherein the antibodies are neutralizing antibodies.

51. The method of any one of claims 48-50, wherein the dosing regimen of the one or more immune suppression agents of the immune suppression regimen includes a dosing regimen of one or more of:
(i) an interleukin-1 (IL-1) signal inhibitor, optionally wherein the IL-1 signal inhibitor is anakinra;
(ii) an IL-6 signal inhibitor, optionally wherein the IL-6 signal inhibitor is tocilizumab;
(iii) a corticosteroid, optionally wherein the corticosteroid is dexamethasone; and
(iv) a calcineurin inhibitor, optionally wherein the calcineurin inhibitor is tacrolimus.
